(19) United States
(12) Patent Application Publication
(10) Pub. No.: US 2023/0323375 A1
(43) Pub. Date: Oct. 12, 2023

(54) SOYBEANS HAVING LOW TRYPSIN INHIBITOR EXPRESSION OR ACTIVITY

(71) Applicant: VIRGINIA POLYTECHNIC INSTITUTE AND STATE UNIVERSITY, Blacksburg, VA (US)

(72) Inventors: Bo Zhang, Blacksburg, VA (US); Bingyu Zhao, Blacksburg, VA (US); Zhibo Wang, Blacksburg, VA (US)

(21) Appl. No.: 18/298,082

(22) Filed: Apr. 10, 2023

Related U.S. Application Data
(60) Provisional application No. 63/329,178, filed on Apr. 8, 2022.

(51) Int. Cl. C12N 15/82; C12N 9/22; A01H 6/54

(52) U.S. Cl. CPC C12N 15/8216; C12N 2310/20; A01H 6/542; C12N 9/22

(57) ABSTRACT

Described in several example embodiments herein are soybean plants and soybeans having one or more KTI genes having reduced or eliminated expression and/or activity thereby resulting soybean plants and soybeans having reduced or eliminated trypsin inhibition. In some embodiments, the soybean plants and soybeans are engineered to have one or more modified KTI genes. Also described herein are methods of making, growing, and using the soybean plants and soybeans described herein.

### CROSS-REFERENCE TO RELATED APPLICATIONS

This application claims the benefit of and priority to U.S. Provisional Patent Application No. 63/329,178, filed on Apr. 8, 2022, entitled “SOYBEANS HAVING LOW TRYPSIN INHIBITOR EXPRESSION OR ACTIVITY,” the contents of which is incorporated by reference herein in its entirety.

### SEQUENCE LISTING

This application contains a sequence listing filed in electronic form as an xml file entitled “VTIP-0360US_ST26.xml”, created on Apr. 10, 2023, and having a size of 52,557 bytes. The content of the sequence listing is incorporated herein in its entirety.

### TECHNICAL FIELD

The subject matter disclosed herein is generally directed to modified soybean plants, and more specifically plants expressing modified trypsin inhibitor genes and gene products.

### BACKGROUND

Soybean is the one of the most important protein sources for animal feed and human consumption. However, the digestibility of soybean meal is severely impacted by anti-nutritional factors in seeds. As such there exists a need for soybeans with an improved nutritional profile. Among these factors, trypsin inhibitor (TI) restrains the function of trypsin, causing low protein digestibility when the raw soybeans are fed to animals. Therefore, soybean meal is routinely heat-treated to inactivate inhibitors, which is energy-intensive and costly, and can degrade certain essential amino acids. During past two decades, although a couple of soybean accessions with natural TI mutations have been introduced to the soybean gene pool, the multiple members of TI genes and lacking of their molecular markers hinder the breeding success of low TI or TI free soybean cultivars.

Citation or identification of any document in this application is not an admission that such a document is available as prior art to the present invention.

### SUMMARY

Described herein are engineered soybean plants and/or soybeans comprising one or more modified Kunitz TI (KTI) genes thereby reducing or eliminating the expression of, amount of, and/or activity of the one or more modified KTI genes and/or gene products as compared to a wild-type or conventionally cultivated soybean plant and/or soybean therefrom.

In certain example embodiments, the one or more modified KTI genes are a modified KTI 1 gene, a modified KTI 3 gene, or both.

In certain example embodiments, the one or more modified KTI genes comprises a modified KTI3. In certain example embodiments, the one or more modified KTI genes consists of a modified KTI3.

In certain example embodiments, the one or more modified KTI genes comprises a modified KTI1 gene comprising SEQ ID NO: 5. In certain example embodiments, the one or more modified KTI genes comprises a modified KTI3 gene comprising one of SEQ ID NO: 6-9 or 49.

In certain example embodiments, the one or more modified KTI genes comprises a modified KTI1 gene and a modified KTI3 gene.

In certain example embodiments, the engineered soybean plant, soybean, or both is heterozygous for the one or more modified KTI genes.

In certain example embodiments, the engineered soybean plant, soybean, or both is homozygous for the one or more modified KTI genes.

In certain example embodiments, the expression of, amount of, and/or activity of the one or more modified KTI genes and/or gene products is decreased 0.1-1,000 fold or more. In certain example embodiments, the expression of, amount of, and/or activity of the one or more modified KTI genes and/or gene products is reduced to below detectable levels.

In certain example embodiments, the expression of, amount of, and/or activity of the one or more modified KTI genes and/or gene products is the engineered soybean plant, soybean, or both has reduced trypsin inhibitor amount and/or activity as compared to a suitable control.

Described in certain example embodiments herein are methods comprising growing, harvesting, or otherwise cultivating an engineered soybean plant and/or soybean of the present description herein.

Described in certain example embodiments herein are methods of making an engineered soybean plant and/or soybean of the present description herein, wherein the method comprises a gene editing technique, optionally a CRISPR-Cas mediated gene editing technique.

Described in certain example embodiments herein are feed and/or food products comprising a soybean plant and/or soybean of the present description herein or a soybean product produced therefrom.

Described in certain example embodiments herein are methods comprising feeding a feed and/or food product of the present description herein to a human or non-human animal.

Described in certain example embodiments herein are kits for identifying soybean plants having reduced trypsin inhibition as compared to a suitable control, the kit comprising (a) a set of primers configured to amplify a region of a KTI1 mutant gene, (b) a set of primers configured to amplify a region of a KTI3 mutant gene, or (c) both (a)-(b), and a set of primers configured to amplify a region of a wild-type or control soybean KTI1 gene, a set of primers configured to amplify a region of a wild-type or control soybean KTI3 gene, or both, wherein the amplicon generated from the set of primers in (a) is different in size and/or sequence as compared to the amplicon generated from the set of primers configured to amplify a region of a wild-type or control soybean KTI1 gene, and wherein the amplicon generated from the set of primers in any one of (b) are different in size and/or sequence as compared to the amplicon generated from the set of primers configured to amplify a region of a wild-type or control soybean KTI3 gene.

In certain example embodiments, the method further comprises one or more reagents suitable for isolating, preparing, amplifying, and/or sequencing nucleic acids.

In certain example embodiments, the wild-type soybean KTI1 gene has a sequence according to SEQ ID NO: 1, the wild-type soybean KTI3 gene has a sequence according to SEQ ID NO: 3.

In certain example embodiments, the set of primers of (a) and the set of primers configured to amplify a region of a wild-type or control soybean KTI1 gene share a common forward primer or a common reverse primer, wherein the set of primers of (b) and the set of primers configured to amplify a region of a wild-type or control soybean KTI3 gene share a common forward primer or a common reverse primer, or both.

In certain example embodiments, the kit comprises one or more primers each having a sequences according to one of SEQ ID NO: 26-30.

Described in several example embodiments herein are engineered soybean plants and/or soybeans comprising one or more modified Kunitz TI (KTI) genes thereby reducing or eliminating the expression of, amount of, and/or activity of the one or more modified KTI genes and/or gene products as compared to a wild-type or conventionally cultivated soybean plant and/or soybean therefrom. In certain example embodiments, the one or more modified KTI genes are KTI 1, KTI 3, or both. In certain example embodiments, expression of, amount of, and/or activity of the one or more modified KTI genes and/or gene products is decreased 0.1-1000 fold or more.

Described in certain example embodiments are soybean plants and/or soybeans comprising a KTI1 and/or KTI3 gene having reduced expression and/or activity, thereby reducing trypsin inhibitor amount and/or activity in the soybean plant and/or soybean.

Described in certain example embodiments are methods growing, harvesting, or otherwise cultivating an engineered soybean plant and/or soybean described herein.

Described in certain example embodiments are of making an engineered soybean plants and/or soybeans described herein, wherein the method comprises a gene editing technique, optionally a CRISPR-Cas mediated gene editing technique.

Described in certain example embodiments are feeds and food products comprising a soybean plant and/or soybean described herein or a soybean product produced therefrom.

Described in certain example embodiments are methods of feeding a feed and/or food product as described herein to a human or non-human animal.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Physical mapping of 38 GmKTI genes on soybean 20 chromosomes. The gene map showing locations of KTI genes on soybean chromosomes was made using Mapinspect. As displayed in the map, 38 KTI genes are located on 9 out of 20 chromosomes.

FIG. 2A-2B—Expression levels of KTI genes in WM82. (FIG. 2A) RNA sequencing data of 38 KTI genes in 26 different tissue types of cv. Williams 82 acquired from Phytozome soybean database was used to construct the heatmap to visualize their expression patterns. (FIG. 2B) The expressions of four soybean KTI genes were monitored by real-time PCR. Samples of leaf, flower, pod, and seed tissues from 2 breeding lines, V98-9005 (normal-TI line) and V03-5903 (low-TI line), were collected for RNA extraction. After reverse transcription, real-time PCR was used to evaluate the expressions of 4 genes including Gm01g095000, Gm08g341000, Gm08g342300, and Gm08g341500 in different tissues with the ELF1B as the reference gene. The expression data was normalized as ACT and shown as mean±s.e. Experiments were repeated three times and obtained similar results.

FIG. 3A-3E—The scheme of binary vector used for CRISPR/Cas9 mediated gene editing on KTI1/KTI3, and transgenes and gene editing have been detected in the leaves of four TO soybean plants. (FIG. 3A) The CRISPR/Cas9 construct harbors three necessary elements exhibited as below: the selection cassette consists of MAS promoter, Bar gene (soybean transformation selection marker), and MAS terminator; the Cas9 cassette consists of U10 promoter, Cas9 gene, and OCS terminator; three guide RNA cassettes and each of them consists of a U6 promoter, and one sgRNA. (FIG. 3B) (SEQ ID NO: 31-33) The sequences of three sgRNAs is shown here. Two sgRNAs were designed, synthesized, and assembled to the plasmid to target on KTI1, while one sgRNA was designed, synthesized, assembled to the plasmid to target on KTI3. The fragments of two transgenes, (FIG. 3C) Cas9 and (FIG. 3D) Bar, have both been detected in lines #2, #5, #11, and #17 by PCR, but not lines #4 and #7. The WM82 gDNA serves as the template for negative control, while the plasmid DNA serves as the template for positive control. (FIG. 3E) (SEQ ID NO: 34-35) The gene editing on KTI1 and KTI3 has also been observed in the leaf tissues of plants at TO generation. The double peak sequence around the sgRNA region indicates the gene editing was ongoing but not completed.

FIG. 4A-4H—Gene editing on KTI1 has been completed for all seeds of TO generation while it has been completed on KTI3 for some seeds of TO generation. From each transgenic line (#2, #5, #11 and #17), four seeds of TO generation were selected randomly for genotyping. (FIG. 4A) (SEQ ID NO: 36-37) The alignment of mutant kti1 in T0 seeds and T1 plant (#5-26) leaf, where the wild type KTI1 in WM82 was the control. (FIG. 4B) Gel electrophoresis of kti1 PCR products showed 16 seeds from line #2, #5, #11, and #17 had the same mutant on kti1, in which 66 nucleotides are lost between two sgRNAs. (FIG. 4C) (SEQ ID NO: 38) Sanger sequencing result displayed the identical mutant kti1. (FIG. 4D) (SEQ ID NO: 39-44) The alignment of mutant kti3 in T0 seeds (#2-3, #5-4, #5-26, #11-2 and #11-4) and T1 plant (#5-26) leaf, where the wild type KTI1 in WM82 was the control. (FIG. 4E) (SEQ ID NO: 45), (FIG. 4F) (SEQ ID NO: 46), (FIG. 4G) (SEQ ID NO: 47), (FIG. 4H) (SEQ ID NO: 48) showed the sanger sequencing results of kti3 mutant in #2-3, #5-4, #11-2, #11-4, and #5-26.

FIG. 5—KTI content declined dramatically in gene-edited seeds. KTI content was measured in 4 double mutated seeds (#2-3, #5-4, #11-2, and #11-4) and 4 seeds with a single mutation on KTI1 (#2-1, #5-1, #11-1, and #17-1), where the KTI content in WM82 seed served as the control. Experiments were conducted with three technical replicates and showed comparable results, shown as mean±s.e. Different letters indicate significant differences.

FIG. 6—TIA declined dramatically in the gene-edited seeds. Bovine trypsin enzyme activities were measured using crude extracts of 4 double mutated seeds (#2-3, #5-4, #11-2, and #11-4), 4 seeds with a single mutation on KTI1 (#2-1, #5-1, #11-1, and #17-1), and WM82. Experiments were conducted with three technical replicates and showed comparable results, shown as mean±s.e. Different letters indicate significant differences.

FIG. 7A-7B—The development of selection markers for breeding low KTI soybean varieties based on the kti1 and kti3 mutants generated by CRISPR/Cas9-mediated gene editing.

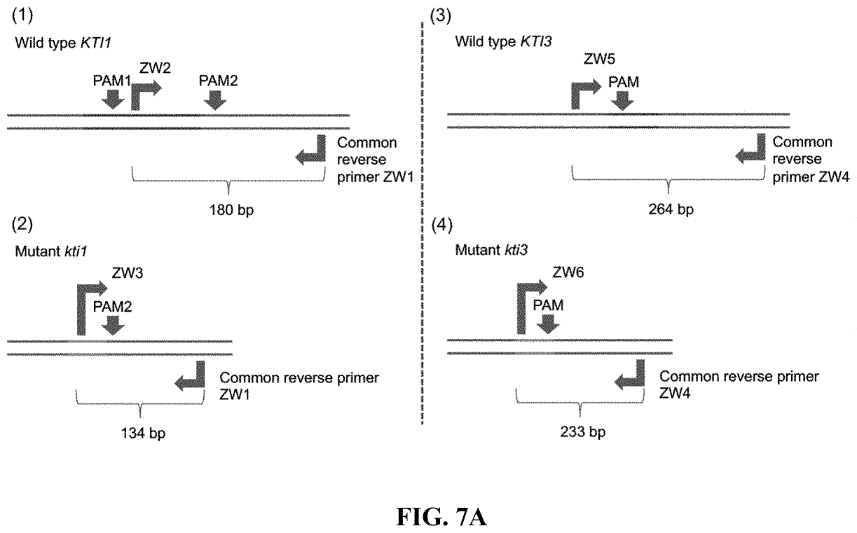

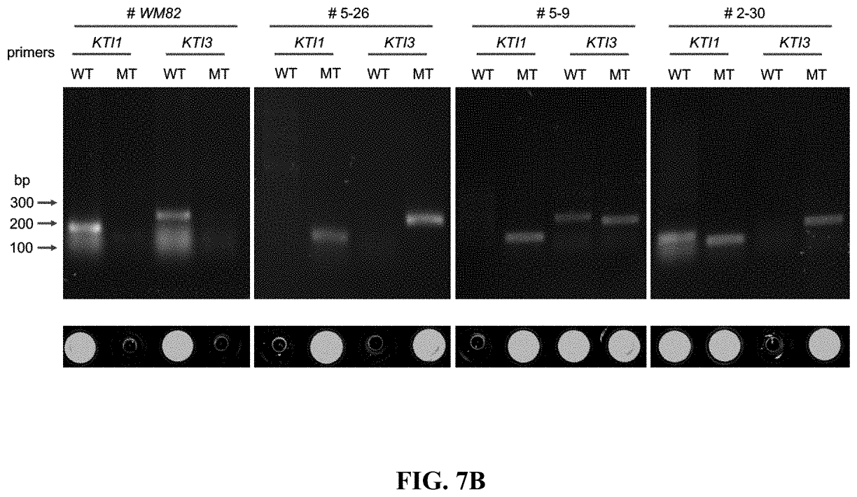

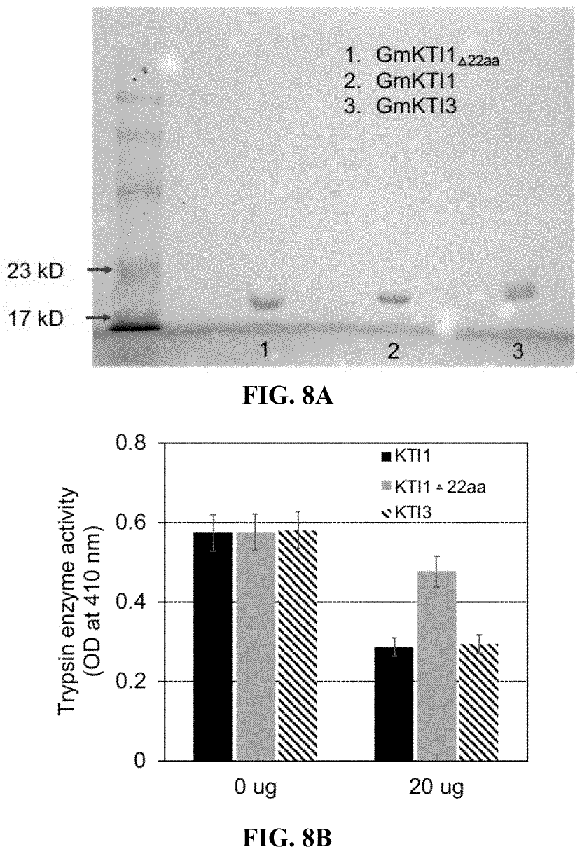

(FIG. 7A) Schematic development of primers for amplification of wild type KTI1 (1), mutant KTI1 (2), wild type KTI3 (3), and mutant KTI3 (4). The red lines indicate the lost fragment in KTI1 or KTI3 during gene editing. The green lines indicate new DNA regions in kti1 or kti3 generated by splicing two fragments. (FIG. 7B) The 4 pairs of primers in (FIG. 7A) were utilized to amplify the alleles of KTI1, kti1, KTI3, and kti3 with gDNA of four different soybean genotypes, including WM82, three transgenic lines #5-26, #5-9, and #2-30. Based on our genotyping data, #5-26 has homozygous mutations of kti1 and kti3; #5-9 only has a homozygous mutation of kti1 but carries the heterozygous mutation of kti3; #2-30 only has a homozygous mutation of kti3 but carries the heterozygous mutation of kti1. Thus, it was clear that the pair of ZW1/ZW2 can amplify wild type KTI1 from WM82 and #2-30 gDNA in PCR tests, while the pair of ZW1/ZW3 can amplify mutant kti1 from #5-9 and #5-26 gDNA. Also, the pair of ZW4/ZW5 can amplify wild type KTI3 from WM82 and #5-9 gDNA, while ZW4/ZW6 can amplify mutant kti3 from #2-30 and #5-26 gDNA. As shown in the bottom panel, only the positive PCR products incubated with the dye of sybrgreen at 75° C. can display the fluorescent signals, suggesting the reliability of the developed gel-electrophoresis-free method for screening mutant alleles of kti1 and kti3.

FIG. 8A-8B—SDS-PAGE of purified recombinant proteins and in-frame mutated protein of KTI1Δ22aa nearly lost the TIA. (FIG. 8A) SDS-PAGE was used to assess the purity of three recombinant proteins, KTI1, KTI3, and KTIΔ22aa. (FIG. 8B) Purified proteins of KTI1 and KTI3, but not KTI1Δ22aa were able to inhibit the trypsin activity in vivo. Experiments were conducted with three technical replicates and obtained similar results.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W.H. Freeman.

Definitions of common terms, analysis, and techniques in genetics can be found in e.g., Hartl and Clark. Principles of Population Genetics. 4^{th} Ed. 2006, published by Oxford University Press. Published by Booker. Genetics: Analysis and Principles, 7^{th} Ed. 2021, published by McGraw Hill; Isik et la., Genetic Data Analysis for Plant and Animal Breeding. First ed. 2017. published by Springer International Publishing AG; Green, E. L. Genetics and Probability in Animal Breeding Experiments. 2014, published by Palgrave; Bourdon, R. M. Understanding Animal Breeding. 2000 2^{nd} Ed. published by Prentice Hall; Pal and Chakravarty. Genetics and Breeding for Disease Resistance of Livestock. First Ed. 2019, published by Academic Press; Fasso, D. Classification of Genetic Variance in Animals. First Ed. 2015, published by Callisto Reference; Megahed, M. Handbook of Animal Breeding and Genetics, 2013, published by Omniscriptum Gmbh & Co. Kg., LAP Lambert Academic Publishing; Reece. Analysis of Genes and Genomes. 2004, published by John Wiley & Sons. Inc; Deonier et al., Computational Genome Analysis. 5^{th} Ed. 2005, published by Springer-Verlag, New York; Meneely, P. Genetic Analysis: Genes, Genomes, and Networks in Eukaryotes. 3^{rd} Ed. 2020, published by Oxford University Press.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g., 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

As used herein, “deoxyribonucleic acid (DNA)” and “ribonucleic acid (RNA)” can generally refer to any polyribonucleotide or polydeoxyribonucleotide (collectively polynucleotides), which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA) or coding mRNA (messenger RNA).

As used herein, “differentially expressed,” refers to the differential production of RNA, including but not limited to mRNA, tRNA, miRNA, siRNA, snRNA, and piRNA transcribed from a gene or regulatory region of a genome or the protein product encoded by a gene as compared to the level of production of RNA or protein by the same gene or regulator region in a normal or a control cell. In another context, “differentially expressed,” also refers to nucleotide sequences or proteins in a cell or tissue which have different temporal and/or spatial expression profiles as compared to a normal or control cell.

As used herein, the term “encode” refers to principle that DNA can be transcribed into RNA, which can then be translated into amino acid sequences that can form proteins.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein, “gene” refers to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA. As such, “gene” refers to stretches of DNA or RNA that encode a polypeptide or an RNA chain that has functional role to play in an organism and hence is the molecular unit of heredity in living organisms. For the purpose of this invention, it may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. In some aspects, a gene can be transcribed to yield non-coding RNA, such that the RNA has a functional role to play in the organism.

As used herein, “gene product” refers to any polynucleotide or polypeptide produced directly or indirectly from transcription of a gene.

As used herein, “mammal,” for the purposes of treatments, can refer to any animal classified as a mammal, including human, domestic and farm animals, nonhuman primates, and zoo, sports, or pet animals, such as, but not limited to, dogs, horses, cats, and cows.

As used herein, “modify” or “modification” broadly denotes a qualitative and/or quantitative alteration, change or variation in that which is being modified. Where modification can be assessed quantitatively—for example, where modification comprises or consists of a change in a quantifiable variable such as a quantifiable property of a cell (e.g., gene and/or protein expression) or where a quantifiable variable provides a suitable surrogate for the modification—modification specifically encompasses both increase (e.g., activation) or decrease (e.g., inhibition) in the measured variable. The term encompasses any extent of such modification, e.g., any extent of such increase or decrease, and may more particularly refer to statistically significant increase or decrease in the measured variable. By means of example, in aspects modification can encompass an increase in the value of the measured variable by about 10 to 500 percent or more. In some embodiments, modification can encompass an increase in the value of at least 10%, 20%, 30%, 40%, 50%, 75%, 100%, 150%, 200%, 250%, 300%, 400% to 500% or more, compared to a reference situation, wild-type, or suitable control without said modification. In aspects, modification may encompass a decrease or reduction in the value of the measured variable by about 5 to about 100%. In some embodiments, the decrease can be about 5%, 10%, 20%, 30%, 40%, 50%, 60%, 70%, 80%, 90%, 95%, 96%, 97%, 98%, 99% to about 100%, compared to a reference situation or suitable control without said modulation. In aspects, modulation may be specific or selective, hence, one or more desired phenotypic aspects of a cell or cell population may be modulated without substantially altering other (unintended, undesired) phenotypic aspect(s).

As used herein, “negative control” can refer to a “control” that is designed to produce no effect or result, provided that all reagents are functioning properly and that the experiment is properly conducted. Other terms that are interchangeable with “negative control” include “sham,” “placebo,” and “mock.”

As used herein, “non-human animal” and likewise: non-human mammal” refers to any animal or mammal, respectively, that is not a human.

As used herein, “positive control” refers to a “control” that is designed to produce the desired result, provided that all reagents are functioning properly and that the experiment is properly conducted.

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body's cells, tissues, and organs.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein “decreased expression”, “reduced expression”, or “underexpression” refers to a reduced or decreased expression of a gene, such as a gene relating to an antigen processing pathway, or a gene product thereof in sample as compared to the expression of said gene or gene product in a suitable control. As used throughout this specification, “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed. In one embodiment, said control is a sample from a healthy individual or otherwise normal individual. By way of a non-limiting example, if said sample is a sample of a lung tumor and comprises lung tissue, said control is lung tissue of a healthy individual. The term “reduced expression” preferably refers to at least a 25% reduction, e.g., at least a 30%, 40%, 50%, 60%, 70%, 75%, 80%, 85%, 90%, 95%, 98% or 99% reduction, relative to such control.

The term “modification causing said reduced expression” refers to a modification in a gene which affects the expression level of that or another gene such that the expression level of that or another gene is reduced or decreased. In particular embodiments, the modification is in a gene relating to an antigen processing pathway. In some embodiments, the modification is in a gene relating to the cross-presentation pathway. Said modification can be any nucleic acid modification including, but not limited to, a mutation, a deletion, an insertion, a replacement, a ligation, a digestion, a break and a frameshift. Said modification is preferably selected from the group consisting of a mutation, a deletion and a frameshift. In particular embodiments, the modification is a mutation which results in reduced expression of the functional gene product.

A “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed.

As used herein, “wild-type” is the average form of an organism, variety, strain, gene, protein, or characteristic as it occurs in a given population in nature, as distinguished from mutant forms that may result from selective breeding, recombinant engineering, and/or transformation with a transgene.

In general, the term “plant” relates and refers to any various photosynthetic, eukaryotic, unicellular or multicellular organism of the kingdom Plantae characteristically growing by cell division, containing chloroplasts, and having cell walls comprised of cellulose. The term plant encompasses monocotyledonous and dicotyledonous plants. The term plant also encompasses offspring, cuttings, grafts, and the like.

As used herein, the term “specific binding” refers to covalent or non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸M or less, 10⁻⁹ M or less, 10⁻¹⁰ M or less, 10⁻¹¹M or less, or 10⁻¹²M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used herein, “specifically detecting” refers to detecting a target polynucleotide predominantly while not substantially detecting non-target sequences at a level that detection (if any) of non-target sequences is undetectable or below the limit of detection or within background level of amplification for the particular method used.

As used herein, “specifically amplifying” refers to amplifying a target polynucleotide predominantly while not substantially amplifying to non-target sequences to a level that amplification (if any) of non-target sequences is undetectable or below the limit of detection or within background level of amplification for the particular method used.

As used herein, the term of art “primer” refers to a nucleic acid sequence that provides a starting point for DNA or RNA synthesis. They can be used in an in vitro reaction, such as in a DNA amplification method such as the polymerase chain reaction, a sequencing method, or in vitro transcription method. Primers are generally short (e.g., about 1-50 nucleotides) sequences, and are typically oligonucleotides. “Primer pair” refers to two primers that are designed to work together in a DNA synthesis and/or amplification method and can define the boundaries of a DNA (or RNA) being synthesized. Typically, there is a forward and reverse primer in a primer pair. Primers have complimentary sequences to a target polynucleotide. Generally, primers have a high degree of complementarity to a region in a target polynucleotide. This region is also referred to in the art as a “primer binding site.” The degree of complementarity between primer to a target polynucleotide can range from about 80-100%, such as 80 to 81, 82, 83, 84, 85, 86, 87, 88, 89, 90, 91, 92, 93, 94, 95, 96, 97, 98, 99, or 100%. The degree of complementarity between a primer to a target polynucleotide can be 81, 82, 83, 84, 85, 86, 87, 88, 89, 90, 91, 92, 93, 94, 95, 96, 97, 98, 99, or 100%.

As used herein, the term of art “amplification” refers to the production of additional polynucleotides via a method employing one or more primers and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. An exemplary amplification method is a PCR method.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Soybean is the one of the most important protein sources for animal feed and human consumption. However, the digestibility of soybean meal is severely impacted by anti-nutritional factors in seeds. Among the anti-nutritional factors, trypsin inhibitor (TI) restrains the function of trypsin, causing low protein digestibility when the raw soybeans are fed to animals. Therefore, soybean meal is routinely heat-treated to inactivate inhibitors, which is energy-intensive and costly, and can degrade certain essential amino acids. During past two decades, although a couple of soybean accessions with natural TI mutations have been introduced to the soybean gene pool, the multiple members of TI genes and a lack of knowing their molecular markers hinder the breeding success of low TI or TI free soybean cultivars.

With the aforementioned deficiencies of current soybeans in mind, described in exemplary embodiments herein are soybean plants, progeny thereof, and soybeans having one or more KTI genes having reduced or eliminated expression and/or activity thereby resulting soybean plants and soybeans having reduced or eliminated trypsin inhibition. In some embodiments, the soybean plants and soybeans are engineered to have one or more modified KTI genes. In some embodiments, the soybean plants, and progeny thereof, soybeans, and products thereof are engineered to express one or more modified KTI genes and/or gene products, such as KTI1 and/or KTI3. In some embodiments, the soybean plants and/or soybeans of the present description have decreased (e.g., by 0.001 fold to 1,000 fold or more) or eliminated trypsin inhibitor gene expression and/or activity, gene product expression and/or activity. In some embodiments, the reduction in one or more TI gene or gene product expression and/or activity of the engineered soybean plants and/or soybeans is greater than conventionally bred cultivars with reduced TI. In some embodiments, the soybean plants and/or soybeans of the present description (including the engineered soybean plants and soybeans) contain one or more molecular markers as identified in at least the Working Examples herein. In some embodiments, the soybean plants and/or soybeans of the present description have reduced anti-nutritional factors and thus improved nutritional value as compared to wild-type or other non-engineered or conventionally bred cultivars with reduced TI.

Also described in several example embodiments herein are methods of growing, harvesting, and/or otherwise cultivating the soybean plants and/or soybeans of the present disclosure. Also described in several example embodiments herein are methods of making an engineered soybean plant and/or soybean of the present description, wherein the method comprises a gene editing technique, optionally a CRISPR-Cas mediated gene editing technique. Also described in several example embodiments herein are feed or food products comprising a soybean plant, soybeans, or a soybean product produced therefrom of the present description. Also described herein are methods of feeding a feed or food product a soybean plant, soybeans, or a soybean product produced therefrom of the present description to a human or non-human animal, including, but not limited to, mammals, avians, reptiles, fish, crustaceans, and/or the like.

Additionally, through the examination of the modified soybeans of the present disclosure, Applicant has identified biomarkers for identifying low TI soybean plants as well as assays that can be used to determine if the biomarkers are present that can be useful in plant selection strategies.

Other compositions, compounds, methods, features, and advantages of the present disclosure will be or become apparent to one having ordinary skill in the art upon examination of the following drawings, detailed description, examples, and/or appendices. It is intended that all such additional compositions, compounds, methods, features, and advantages be included within this description, and be within the scope of the present description.

### Engineered Plants and Products Produced Therefrom

Described herein are engineered soybean plants and/or soybeans comprising one or more modified KTI genes thereby reducing or eliminating the expression of, amount of, and/or activity of the one or more modified KTI genes and/or gene products as compared to a wild-type or conventionally cultivated soybean plant and/or soybean therefrom. In some embodiments, the one or more KTI gene products are one or more KTI mRNA, one or more KTI polypeptides, or both.

In some embodiments, the one or more modified KTI genes includes or is a modified KTI1 gene. In some embodiments, the one or more modified KTI genes includes or is a modified KTI3 gene. In In certain example embodiments, the one or more modified KTI genes comprises a modified KTI1 gene and a modified KTI3 gene. In certain example embodiments, the engineered soybean plant, soybean, or both is heterozygous for the one or more modified KTI genes. In certain example embodiments, the engineered soybean plant, soybean, or both is homozygous for the one or more modified KTI genes.

In certain example embodiments, the one or more KTI genes includes or is a modified KTI1 having a sequence that is about 80%-100% identical to SEQ ID NO: 5 or a region thereof of at least 10 contiguous nucleotides. In certain example embodiments, the one or more KTI genes includes or is a modified KTI1 having a sequence that is about 80%, to/or 81%, 82%, 83%, 84%, 85%, 86%, 87%, 88%, 89%, 90%, 91%, 92%, 93%, 94%, 95%, 96%, 97%, 98%, 99%, 100% identical to SEQ ID NO: 5 or a region thereof of at least 10 contiguous nucleotides. In certain example embodiments, the one or more modified KTI genes includes a modified KTI1 gene having or composed of a sequence according to SEQ ID NO: 5 or a region thereof of at least 10 contiguous nucleotides.

In certain example embodiments, the one or more KTI genes includes or is a modified KTI3 having a sequence that is about 80%-100% identical to any one of SEQ ID NO: 6-9 or 49 or a region thereof of at least 10 contiguous nucleotides. In certain example embodiments, the one or more KTI genes includes or is a modified KTI3 having a sequence that is about 80%, to/or 81%, 82%, 83%, 84%, 85%, 86%, 87%, 88%, 89%, 90%, 91%, 92%, 93%, 94%, 95%, 96%, 97%, 98%, 99%, 100% identical to any one of SEQ ID NO: 6-9 or 49 or a region thereof of at least 10 contiguous nucleotides. In certain example embodiments, the one or more modified KTI genes includes a modified KTI3 gene having or composed of a sequence according to one of SEQ ID NO: 6-9 or 49 or a region thereof of at least 10 contiguous nucleotides.

In certain example embodiments, the expression of, amount of, and/or activity of the one or more modified KTI genes and/or gene products is decreased 0.1-1,000 fold or more as compared to a suitable control, for example a non-engineered, unmodified, or wild-type control. In certain example embodiments, the expression of, amount of, and/or activity of the one or more modified KTI genes and/or gene products is reduced to below detectable levels. In some embodiments, the expression of, amount of, and/or activity of the one or more modified KTI genes and/or gene products is reduced by a non-zero number to/or 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20, 20.1, 20.2, 20.3, 20.4, 20.5, 20.6, 20.7, 20.8, 20.9, 21, 21.1, 21.2, 21.3, 21.4, 21.5, 21.6, 21.7, 21.8, 21.9, 22, 22.1, 22.2, 22.3, 22.4, 22.5, 22.6, 22.7, 22.8, 22.9, 23, 23.1, 23.2, 23.3, 23.4, 23.5, 23.6, 23.7, 23.8, 23.9, 24, 24.1, 24.2, 24.3, 24.4, 24.5, 24.6, 24.7, 24.8, 24.9, 25, 25.1, 25.2, 25.3, 25.4, 25.5, 25.6, 25.7, 25.8, 25.9, 26, 26.1, 26.2, 26.3, 26.4, 26.5, 26.6, 26.7, 26.8, 26.9, 27, 27.1, 27.2, 27.3, 27.4, 27.5, 27.6, 27.7, 27.8, 27.9, 28, 28.1, 28.2, 28.3, 28.4, 28.5, 28.6, 28.7, 28.8, 28.9, 29, 29.1, 29.2, 29.3, 29.4, 29.5, 29.6, 29.7, 29.8, 29.9, 30, 30.1, 30.2, 30.3, 30.4, 30.5, 30.6, 30.7, 30.8, 30.9, 31, 31.1, 31.2, 31.3, 31.4, 31.5, 31.6, 31.7, 31.8, 31.9, 32, 32.1, 32.2, 32.3, 32.4, 32.5, 32.6, 32.7, 32.8, 32.9, 33, 33.1, 33.2, 33.3, 33.4, 33.5, 33.6, 33.7, 33.8, 33.9, 34, 34.1, 34.2, 34.3, 34.4, 34.5, 34.6, 34.7, 34.8, 34.9, 35, 35.1, 35.2, 35.3, 35.4, 35.5, 35.6, 35.7, 35.8, 35.9, 36, 36.1, 36.2, 36.3, 36.4, 36.5, 36.6, 36.7, 36.8, 36.9, 37, 37.1, 37.2, 37.3, 37.4, 37.5, 37.6, 37.7, 37.8, 37.9, 38, 38.1, 38.2, 38.3, 38.4, 38.5, 38.6, 38.7, 38.8, 38.9, 39, 39.1, 39.2, 39.3, 39.4, 39.5, 39.6, 39.7, 39.8, 39.9, 40, 40.1, 40.2, 40.3, 40.4, 40.5, 40.6, 40.7, 40.8, 40.9, 41, 41.1, 41.2, 41.3, 41.4, 41.5, 41.6, 41.7, 41.8, 41.9, 42, 42.1, 42.2, 42.3, 42.4, 42.5, 42.6, 42.7, 42.8, 42.9, 43, 43.1, 43.2, 43.3, 43.4, 43.5, 43.6, 43.7, 43.8, 43.9, 44, 44.1, 44.2, 44.3, 44.4, 44.5, 44.6, 44.7, 44.8, 44.9, 45, 45.1, 45.2, 45.3, 45.4, 45.5, 45.6, 45.7, 45.8, 45.9, 46, 46.1, 46.2, 46.3, 46.4, 46.5, 46.6, 46.7, 46.8, 46.9, 47, 47.1, 47.2, 47.3, 47.4, 47.5, 47.6, 47.7, 47.8, 47.9, 48, 48.1, 48.2, 48.3, 48.4, 48.5, 48.6, 48.7, 48.8, 48.9, 49, 49.1, 49.2, 49.3, 49.4, 49.5, 49.6, 49.7, 49.8, 49.9, 50, 50.1, 50.2, 50.3, 50.4, 50.5, 50.6, 50.7, 50.8, 50.9, 51, 51.1, 51.2, 51.3, 51.4, 51.5, 51.6, 51.7, 51.8, 51.9, 52, 52.1, 52.2, 52.3, 52.4, 52.5, 52.6, 52.7, 52.8, 52.9, 53, 53.1, 53.2, 53.3, 53.4, 53.5, 53.6, 53.7, 53.8, 53.9, 54, 54.1, 54.2, 54.3, 54.4, 54.5, 54.6, 54.7, 54.8, 54.9, 55, 55.1, 55.2, 55.3, 55.4, 55.5, 55.6, 55.7, 55.8, 55.9, 56, 56.1, 56.2, 56.3, 56.4, 56.5, 56.6, 56.7, 56.8, 56.9, 57, 57.1, 57.2, 57.3, 57.4, 57.5, 57.6, 57.7, 57.8, 57.9, 58, 58.1, 58.2, 58.3, 58.4, 58.5, 58.6, 58.7, 58.8, 58.9, 59, 59.1, 59.2, 59.3, 59.4, 59.5, 59.6, 59.7, 59.8, 59.9, 60, 60.1, 60.2, 60.3, 60.4, 60.5, 60.6, 60.7, 60.8, 60.9, 61, 61.1, 61.2, 61.3, 61.4, 61.5, 61.6, 61.7, 61.8, 61.9, 62, 62.1, 62.2, 62.3, 62.4, 62.5, 62.6, 62.7, 62.8, 62.9, 63, 63.1, 63.2, 63.3, 63.4, 63.5, 63.6, 63.7, 63.8, 63.9, 64, 64.1, 64.2, 64.3, 64.4, 64.5, 64.6, 64.7, 64.8, 64.9, 65, 65.1, 65.2, 65.3, 65.4, 65.5, 65.6, 65.7, 65.8, 65.9, 66, 66.1, 66.2, 66.3, 66.4, 66.5, 66.6, 66.7, 66.8, 66.9, 67, 67.1, 67.2, 67.3, 67.4, 67.5, 67.6, 67.7, 67.8, 67.9, 68, 68.1, 68.2, 68.3, 68.4, 68.5, 68.6, 68.7, 68.8, 68.9, 69, 69.1, 69.2, 69.3, 69.4, 69.5, 69.6, 69.7, 69.8, 69.9, 70, 70.1, 70.2, 70.3, 70.4, 70.5, 70.6, 70.7, 70.8, 70.9, 71, 71.1, 71.2, 71.3, 71.4, 71.5, 71.6, 71.7, 71.8, 71.9, 72, 72.1, 72.2, 72.3, 72.4, 72.5, 72.6, 72.7, 72.8, 72.9, 73, 73.1, 73.2, 73.3, 73.4, 73.5, 73.6, 73.7, 73.8, 73.9, 74, 74.1, 74.2, 74.3, 74.4, 74.5, 74.6, 74.7, 74.8, 74.9, 75, 75.1, 75.2, 75.3, 75.4, 75.5, 75.6, 75.7, 75.8, 75.9, 76, 76.1, 76.2, 76.3, 76.4, 76.5, 76.6, 76.7, 76.8, 76.9, 77, 77.1, 77.2, 77.3, 77.4, 77.5, 77.6, 77.7, 77.8, 77.9, 78, 78.1, 78.2, 78.3, 78.4, 78.5, 78.6, 78.7, 78.8, 78.9, 79, 79.1, 79.2, 79.3, 79.4, 79.5, 79.6, 79.7, 79.8, 79.9, 80, 80.1, 80.2, 80.3, 80.4, 80.5, 80.6, 80.7, 80.8, 80.9, 81, 81.1, 81.2, 81.3, 81.4, 81.5, 81.6, 81.7, 81.8, 81.9, 82, 82.1, 82.2, 82.3, 82.4, 82.5, 82.6, 82.7, 82.8, 82.9, 83, 83.1, 83.2, 83.3, 83.4, 83.5, 83.6, 83.7, 83.8, 83.9, 84, 84.1, 84.2, 84.3, 84.4, 84.5, 84.6, 84.7, 84.8, 84.9, 85, 85.1, 85.2, 85.3, 85.4, 85.5, 85.6, 85.7, 85.8, 85.9, 86, 86.1, 86.2, 86.3, 86.4, 86.5, 86.6, 86.7, 86.8, 86.9, 87, 87.1, 87.2, 87.3, 87.4, 87.5, 87.6, 87.7, 87.8, 87.9, 88, 88.1, 88.2, 88.3, 88.4, 88.5, 88.6, 88.7, 88.8, 88.9, 89, 89.1, 89.2, 89.3, 89.4, 89.5, 89.6, 89.7, 89.8, 89.9, 90, 90.1, 90.2, 90.3, 90.4, 90.5, 90.6, 90.7, 90.8, 90.9, 91, 91.1, 91.2, 91.3, 91.4, 91.5, 91.6, 91.7, 91.8, 91.9, 92, 92.1, 92.2, 92.3, 92.4, 92.5, 92.6, 92.7, 92.8, 92.9, 93, 93.1, 93.2, 93.3, 93.4, 93.5, 93.6, 93.7, 93.8, 93.9, 94, 94.1, 94.2, 94.3, 94.4, 94.5, 94.6, 94.7, 94.8, 94.9, 95, 95.1, 95.2, 95.3, 95.4, 95.5, 95.6, 95.7, 95.8, 95.9, 96, 96.1, 96.2, 96.3, 96.4, 96.5, 96.6, 96.7, 96.8, 96.9, 97, 97.1, 97.2, 97.3, 97.4, 97.5, 97.6, 97.7, 97.8, 97.9, 98, 98.1, 98.2, 98.3, 98.4, 98.5, 98.6, 98.7, 98.8, 98.9, 99, 99.1, 99.2, 99.3, 99.4, 99.5, 99.6, 99.7, 99.8, 99.9, 100, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000 fold or more.

In certain example embodiments, the one or more modified KTI genes are a modified KTI 1 gene, a modified KTI 3 gene, or both. In certain example embodiments, the one or more modified KTI genes includes a modified KTI3. In certain example embodiments, the one or more modified KTI genes is a modified KTI3. In some embodiments, the modified KTI3 gene and/or gene product expression, amount, and/or activity is reduced by a non-zero number to/or 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20, 20.1, 20.2, 20.3, 20.4, 20.5, 20.6, 20.7, 20.8, 20.9, 21, 21.1, 21.2, 21.3, 21.4, 21.5, 21.6, 21.7, 21.8, 21.9, 22, 22.1, 22.2, 22.3, 22.4, 22.5, 22.6, 22.7, 22.8, 22.9, 23, 23.1, 23.2, 23.3, 23.4, 23.5, 23.6, 23.7, 23.8, 23.9, 24, 24.1, 24.2, 24.3, 24.4, 24.5, 24.6, 24.7, 24.8, 24.9, 25, 25.1, 25.2, 25.3, 25.4, 25.5, 25.6, 25.7, 25.8, 25.9, 26, 26.1, 26.2, 26.3, 26.4, 26.5, 26.6, 26.7, 26.8, 26.9, 27, 27.1, 27.2, 27.3, 27.4, 27.5, 27.6, 27.7, 27.8, 27.9, 28, 28.1, 28.2, 28.3, 28.4, 28.5, 28.6, 28.7, 28.8, 28.9, 29, 29.1, 29.2, 29.3, 29.4, 29.5, 29.6, 29.7, 29.8, 29.9, 30, 30.1, 30.2, 30.3, 30.4, 30.5, 30.6, 30.7, 30.8, 30.9, 31, 31.1, 31.2, 31.3, 31.4, 31.5, 31.6, 31.7, 31.8, 31.9, 32, 32.1, 32.2, 32.3, 32.4, 32.5, 32.6, 32.7, 32.8, 32.9, 33, 33.1, 33.2, 33.3, 33.4, 33.5, 33.6, 33.7, 33.8, 33.9, 34, 34.1, 34.2, 34.3, 34.4, 34.5, 34.6, 34.7, 34.8, 34.9, 35, 35.1, 35.2, 35.3, 35.4, 35.5, 35.6, 35.7, 35.8, 35.9, 36, 36.1, 36.2, 36.3, 36.4, 36.5, 36.6, 36.7, 36.8, 36.9, 37, 37.1, 37.2, 37.3, 37.4, 37.5, 37.6, 37.7, 37.8, 37.9, 38, 38.1, 38.2, 38.3, 38.4, 38.5, 38.6, 38.7, 38.8, 38.9, 39, 39.1, 39.2, 39.3, 39.4, 39.5, 39.6, 39.7, 39.8, 39.9, 40, 40.1, 40.2, 40.3, 40.4, 40.5, 40.6, 40.7, 40.8, 40.9, 41, 41.1, 41.2, 41.3, 41.4, 41.5, 41.6, 41.7, 41.8, 41.9, 42, 42.1, 42.2, 42.3, 42.4, 42.5, 42.6, 42.7, 42.8, 42.9, 43, 43.1, 43.2, 43.3, 43.4, 43.5, 43.6, 43.7, 43.8, 43.9, 44, 44.1, 44.2, 44.3, 44.4, 44.5, 44.6, 44.7, 44.8, 44.9, 45, 45.1, 45.2, 45.3, 45.4, 45.5, 45.6, 45.7, 45.8, 45.9, 46, 46.1, 46.2, 46.3, 46.4, 46.5, 46.6, 46.7, 46.8, 46.9, 47, 47.1, 47.2, 47.3, 47.4, 47.5, 47.6, 47.7, 47.8, 47.9, 48, 48.1, 48.2, 48.3, 48.4, 48.5, 48.6, 48.7, 48.8, 48.9, 49, 49.1, 49.2, 49.3, 49.4, 49.5, 49.6, 49.7, 49.8, 49.9, 50, 50.1, 50.2, 50.3, 50.4, 50.5, 50.6, 50.7, 50.8, 50.9, 51, 51.1, 51.2, 51.3, 51.4, 51.5, 51.6, 51.7, 51.8, 51.9, 52, 52.1, 52.2, 52.3, 52.4, 52.5, 52.6, 52.7, 52.8, 52.9, 53, 53.1, 53.2, 53.3, 53.4, 53.5, 53.6, 53.7, 53.8, 53.9, 54, 54.1, 54.2, 54.3, 54.4, 54.5, 54.6, 54.7, 54.8, 54.9, 55, 55.1, 55.2, 55.3, 55.4, 55.5, 55.6, 55.7, 55.8, 55.9, 56, 56.1, 56.2, 56.3, 56.4, 56.5, 56.6, 56.7, 56.8, 56.9, 57, 57.1, 57.2, 57.3, 57.4, 57.5, 57.6, 57.7, 57.8, 57.9, 58, 58.1, 58.2, 58.3, 58.4, 58.5, 58.6, 58.7, 58.8, 58.9, 59, 59.1, 59.2, 59.3, 59.4, 59.5, 59.6, 59.7, 59.8, 59.9, 60, 60.1, 60.2, 60.3, 60.4, 60.5, 60.6, 60.7, 60.8, 60.9, 61, 61.1, 61.2, 61.3, 61.4, 61.5, 61.6, 61.7, 61.8, 61.9, 62, 62.1, 62.2, 62.3, 62.4, 62.5, 62.6, 62.7, 62.8, 62.9, 63, 63.1, 63.2, 63.3, 63.4, 63.5, 63.6, 63.7, 63.8, 63.9, 64, 64.1, 64.2, 64.3, 64.4, 64.5, 64.6, 64.7, 64.8, 64.9, 65, 65.1, 65.2, 65.3, 65.4, 65.5, 65.6, 65.7, 65.8, 65.9, 66, 66.1, 66.2, 66.3, 66.4, 66.5, 66.6, 66.7, 66.8, 66.9, 67, 67.1, 67.2, 67.3, 67.4, 67.5, 67.6, 67.7, 67.8, 67.9, 68, 68.1, 68.2, 68.3, 68.4, 68.5, 68.6, 68.7, 68.8, 68.9, 69, 69.1, 69.2, 69.3, 69.4, 69.5, 69.6, 69.7, 69.8, 69.9, 70, 70.1, 70.2, 70.3, 70.4, 70.5, 70.6, 70.7, 70.8, 70.9, 71, 71.1, 71.2, 71.3, 71.4, 71.5, 71.6, 71.7, 71.8, 71.9, 72, 72.1, 72.2, 72.3, 72.4, 72.5, 72.6, 72.7, 72.8, 72.9, 73, 73.1, 73.2, 73.3, 73.4, 73.5, 73.6, 73.7, 73.8, 73.9, 74, 74.1, 74.2, 74.3, 74.4, 74.5, 74.6, 74.7, 74.8, 74.9, 75, 75.1, 75.2, 75.3, 75.4, 75.5, 75.6, 75.7, 75.8, 75.9, 76, 76.1, 76.2, 76.3, 76.4, 76.5, 76.6, 76.7, 76.8, 76.9, 77, 77.1, 77.2, 77.3, 77.4, 77.5, 77.6, 77.7, 77.8, 77.9, 78, 78.1, 78.2, 78.3, 78.4, 78.5, 78.6, 78.7, 78.8, 78.9, 79, 79.1, 79.2, 79.3, 79.4, 79.5, 79.6, 79.7, 79.8, 79.9, 80, 80.1, 80.2, 80.3, 80.4, 80.5, 80.6, 80.7, 80.8, 80.9, 81, 81.1, 81.2, 81.3, 81.4, 81.5, 81.6, 81.7, 81.8, 81.9, 82, 82.1, 82.2, 82.3, 82.4, 82.5, 82.6, 82.7, 82.8, 82.9, 83, 83.1, 83.2, 83.3, 83.4, 83.5, 83.6, 83.7, 83.8, 83.9, 84, 84.1, 84.2, 84.3, 84.4, 84.5, 84.6, 84.7, 84.8, 84.9, 85, 85.1, 85.2, 85.3, 85.4, 85.5, 85.6, 85.7, 85.8, 85.9, 86, 86.1, 86.2, 86.3, 86.4, 86.5, 86.6, 86.7, 86.8, 86.9, 87, 87.1, 87.2, 87.3, 87.4, 87.5, 87.6, 87.7, 87.8, 87.9, 88, 88.1, 88.2, 88.3, 88.4, 88.5, 88.6, 88.7, 88.8, 88.9, 89, 89.1, 89.2, 89.3, 89.4, 89.5, 89.6, 89.7, 89.8, 89.9, 90, 90.1, 90.2, 90.3, 90.4, 90.5, 90.6, 90.7, 90.8, 90.9, 91, 91.1, 91.2, 91.3, 91.4, 91.5, 91.6, 91.7, 91.8, 91.9, 92, 92.1, 92.2, 92.3, 92.4, 92.5, 92.6, 92.7, 92.8, 92.9, 93, 93.1, 93.2, 93.3, 93.4, 93.5, 93.6, 93.7, 93.8, 93.9, 94, 94.1, 94.2, 94.3, 94.4, 94.5, 94.6, 94.7, 94.8, 94.9, 95, 95.1, 95.2, 95.3, 95.4, 95.5, 95.6, 95.7, 95.8, 95.9, 96, 96.1, 96.2, 96.3, 96.4, 96.5, 96.6, 96.7, 96.8, 96.9, 97, 97.1, 97.2, 97.3, 97.4, 97.5, 97.6, 97.7, 97.8, 97.9, 98, 98.1, 98.2, 98.3, 98.4, 98.5, 98.6, 98.7, 98.8, 98.9, 99, 99.1, 99.2, 99.3, 99.4, 99.5, 99.6, 99.7, 99.8, 99.9, 100, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000 fold or more.

In certain example embodiments, the engineered soybean plant, soybean, or both has reduced trypsin inhibitor amount and/or activity (e.g., trypsin inhibition) as compared to a suitable control, such as a wild-type, unmodified, and/or non-engineered control. In some embodiments the amount of trypsin inhibitor amount and/or activity is reduced by a non-zero number to/or 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20, 20.1, 20.2, 20.3, 20.4, 20.5, 20.6, 20.7, 20.8, 20.9, 21, 21.1, 21.2, 21.3, 21.4, 21.5, 21.6, 21.7, 21.8, 21.9, 22, 22.1, 22.2, 22.3, 22.4, 22.5, 22.6, 22.7, 22.8, 22.9, 23, 23.1, 23.2, 23.3, 23.4, 23.5, 23.6, 23.7, 23.8, 23.9, 24, 24.1, 24.2, 24.3, 24.4, 24.5, 24.6, 24.7, 24.8, 24.9, 25, 25.1, 25.2, 25.3, 25.4, 25.5, 25.6, 25.7, 25.8, 25.9, 26, 26.1, 26.2, 26.3, 26.4, 26.5, 26.6, 26.7, 26.8, 26.9, 27, 27.1, 27.2, 27.3, 27.4, 27.5, 27.6, 27.7, 27.8, 27.9, 28, 28.1, 28.2, 28.3, 28.4, 28.5, 28.6, 28.7, 28.8, 28.9, 29, 29.1, 29.2, 29.3, 29.4, 29.5, 29.6, 29.7, 29.8, 29.9, 30, 30.1, 30.2, 30.3, 30.4, 30.5, 30.6, 30.7, 30.8, 30.9, 31, 31.1, 31.2, 31.3, 31.4, 31.5, 31.6, 31.7, 31.8, 31.9, 32, 32.1, 32.2, 32.3, 32.4, 32.5, 32.6, 32.7, 32.8, 32.9, 33, 33.1, 33.2, 33.3, 33.4, 33.5, 33.6, 33.7, 33.8, 33.9, 34, 34.1, 34.2, 34.3, 34.4, 34.5, 34.6, 34.7, 34.8, 34.9, 35, 35.1, 35.2, 35.3, 35.4, 35.5, 35.6, 35.7, 35.8, 35.9, 36, 36.1, 36.2, 36.3, 36.4, 36.5, 36.6, 36.7, 36.8, 36.9, 37, 37.1, 37.2, 37.3, 37.4, 37.5, 37.6, 37.7, 37.8, 37.9, 38, 38.1, 38.2, 38.3, 38.4, 38.5, 38.6, 38.7, 38.8, 38.9, 39, 39.1, 39.2, 39.3, 39.4, 39.5, 39.6, 39.7, 39.8, 39.9, 40, 40.1, 40.2, 40.3, 40.4, 40.5, 40.6, 40.7, 40.8, 40.9, 41, 41.1, 41.2, 41.3, 41.4, 41.5, 41.6, 41.7, 41.8, 41.9, 42, 42.1, 42.2, 42.3, 42.4, 42.5, 42.6, 42.7, 42.8, 42.9, 43, 43.1, 43.2, 43.3, 43.4, 43.5, 43.6, 43.7, 43.8, 43.9, 44, 44.1, 44.2, 44.3, 44.4, 44.5, 44.6, 44.7, 44.8, 44.9, 45, 45.1, 45.2, 45.3, 45.4, 45.5, 45.6, 45.7, 45.8, 45.9, 46, 46.1, 46.2, 46.3, 46.4, 46.5, 46.6, 46.7, 46.8, 46.9, 47, 47.1, 47.2, 47.3, 47.4, 47.5, 47.6, 47.7, 47.8, 47.9, 48, 48.1, 48.2, 48.3, 48.4, 48.5, 48.6, 48.7, 48.8, 48.9, 49, 49.1, 49.2, 49.3, 49.4, 49.5, 49.6, 49.7, 49.8, 49.9, 50, 50.1, 50.2, 50.3, 50.4, 50.5, 50.6, 50.7, 50.8, 50.9, 51, 51.1, 51.2, 51.3, 51.4, 51.5, 51.6, 51.7, 51.8, 51.9, 52, 52.1, 52.2, 52.3, 52.4, 52.5, 52.6, 52.7, 52.8, 52.9, 53, 53.1, 53.2, 53.3, 53.4, 53.5, 53.6, 53.7, 53.8, 53.9, 54, 54.1, 54.2, 54.3, 54.4, 54.5, 54.6, 54.7, 54.8, 54.9, 55, 55.1, 55.2, 55.3, 55.4, 55.5, 55.6, 55.7, 55.8, 55.9, 56, 56.1, 56.2, 56.3, 56.4, 56.5, 56.6, 56.7, 56.8, 56.9, 57, 57.1, 57.2, 57.3, 57.4, 57.5, 57.6, 57.7, 57.8, 57.9, 58, 58.1, 58.2, 58.3, 58.4, 58.5, 58.6, 58.7, 58.8, 58.9, 59, 59.1, 59.2, 59.3, 59.4, 59.5, 59.6, 59.7, 59.8, 59.9, 60, 60.1, 60.2, 60.3, 60.4, 60.5, 60.6, 60.7, 60.8, 60.9, 61, 61.1, 61.2, 61.3, 61.4, 61.5, 61.6, 61.7, 61.8, 61.9, 62, 62.1, 62.2, 62.3, 62.4, 62.5, 62.6, 62.7, 62.8, 62.9, 63, 63.1, 63.2, 63.3, 63.4, 63.5, 63.6, 63.7, 63.8, 63.9, 64, 64.1, 64.2, 64.3, 64.4, 64.5, 64.6, 64.7, 64.8, 64.9, 65, 65.1, 65.2, 65.3, 65.4, 65.5, 65.6, 65.7, 65.8, 65.9, 66, 66.1, 66.2, 66.3, 66.4, 66.5, 66.6, 66.7, 66.8, 66.9, 67, 67.1, 67.2, 67.3, 67.4, 67.5, 67.6, 67.7, 67.8, 67.9, 68, 68.1, 68.2, 68.3, 68.4, 68.5, 68.6, 68.7, 68.8, 68.9, 69, 69.1, 69.2, 69.3, 69.4, 69.5, 69.6, 69.7, 69.8, 69.9, 70, 70.1, 70.2, 70.3, 70.4, 70.5, 70.6, 70.7, 70.8, 70.9, 71, 71.1, 71.2, 71.3, 71.4, 71.5, 71.6, 71.7, 71.8, 71.9, 72, 72.1, 72.2, 72.3, 72.4, 72.5, 72.6, 72.7, 72.8, 72.9, 73, 73.1, 73.2, 73.3, 73.4, 73.5, 73.6, 73.7, 73.8, 73.9, 74, 74.1, 74.2, 74.3, 74.4, 74.5, 74.6, 74.7, 74.8, 74.9, 75, 75.1, 75.2, 75.3, 75.4, 75.5, 75.6, 75.7, 75.8, 75.9, 76, 76.1, 76.2, 76.3, 76.4, 76.5, 76.6, 76.7, 76.8, 76.9, 77, 77.1, 77.2, 77.3, 77.4, 77.5, 77.6, 77.7, 77.8, 77.9, 78, 78.1, 78.2, 78.3, 78.4, 78.5, 78.6, 78.7, 78.8, 78.9, 79, 79.1, 79.2, 79.3, 79.4, 79.5, 79.6, 79.7, 79.8, 79.9, 80, 80.1, 80.2, 80.3, 80.4, 80.5, 80.6, 80.7, 80.8, 80.9, 81, 81.1, 81.2, 81.3, 81.4, 81.5, 81.6, 81.7, 81.8, 81.9, 82, 82.1, 82.2, 82.3, 82.4, 82.5, 82.6, 82.7, 82.8, 82.9, 83, 83.1, 83.2, 83.3, 83.4, 83.5, 83.6, 83.7, 83.8, 83.9, 84, 84.1, 84.2, 84.3, 84.4, 84.5, 84.6, 84.7, 84.8, 84.9, 85, 85.1, 85.2, 85.3, 85.4, 85.5, 85.6, 85.7, 85.8, 85.9, 86, 86.1, 86.2, 86.3, 86.4, 86.5, 86.6, 86.7, 86.8, 86.9, 87, 87.1, 87.2, 87.3, 87.4, 87.5, 87.6, 87.7, 87.8, 87.9, 88, 88.1, 88.2, 88.3, 88.4, 88.5, 88.6, 88.7, 88.8, 88.9, 89, 89.1, 89.2, 89.3, 89.4, 89.5, 89.6, 89.7, 89.8, 89.9, 90, 90.1, 90.2, 90.3, 90.4, 90.5, 90.6, 90.7, 90.8, 90.9, 91, 91.1, 91.2, 91.3, 91.4, 91.5, 91.6, 91.7, 91.8, 91.9, 92, 92.1, 92.2, 92.3, 92.4, 92.5, 92.6, 92.7, 92.8, 92.9, 93, 93.1, 93.2, 93.3, 93.4, 93.5, 93.6, 93.7, 93.8, 93.9, 94, 94.1, 94.2, 94.3, 94.4, 94.5, 94.6, 94.7, 94.8, 94.9, 95, 95.1, 95.2, 95.3, 95.4, 95.5, 95.6, 95.7, 95.8, 95.9, 96, 96.1, 96.2, 96.3, 96.4, 96.5, 96.6, 96.7, 96.8, 96.9, 97, 97.1, 97.2, 97.3, 97.4, 97.5, 97.6, 97.7, 97.8, 97.9, 98, 98.1, 98.2, 98.3, 98.4, 98.5, 98.6, 98.7, 98.8, 98.9, 99, 99.1, 99.2, 99.3, 99.4, 99.5, 99.6, 99.7, 99.8, 99.9, 100, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000 fold or more.

### Vectors and Vector Systems

Also provided herein are vectors that can contain one or more of the engineered KTI (e.g., KTI1 and KTI3) polynucleotides (also referred to as modified KTI1 and KTI3 genes) described herein. The vectors can be useful in producing, for example, bacterial, plant cells, and/or transgenic (engineered) plants that can contain, replicate, and/or express a modified KTI gene and/or gene product described herein. Within the scope of this disclosure are vectors containing one or more of the polynucleotide sequences described herein. One or more of the polynucleotides that are part of the modified KTI encoding polynucleotides described herein can be included in a vector or vector system. The vectors and/or vector systems can be used, for example, to express one or more of the polynucleotides in a cell, such as a plant cell or agrobacterium or to produce particles such as viral particles that can be used to generate transgenic cells and/or plants. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g. a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other embodiments of the vectors and vector systems are described elsewhere herein.

In some embodiments, the vector can be a bicistronic vector. In some embodiments, a bicistronic vector can be used for expression one or more modified KTI genes or other (e.g., reporter) polynucleotides and/or one or more regions or domains thereof described herein.

### Cell-Based Vector Amplification and Expression

Vectors can be designed for amplification, propagation, and expression of one or more elements of the modified KTI encoding polynucleotides described herein (e.g., nucleic acid transcripts, proteins, enzymes, and combinations thereof) in a suitable host cell. In some embodiments, the suitable host cell is a prokaryotic cell. Suitable host cells include, but are not limited to, bacterial cells, yeast cells, insect cells, mammalian cells, and more particularly plant cells. The vectors can be viral-based or non-viral based. In some embodiments, the suitable host cell is a eukaryotic cell. In some embodiments, the suitable host cell is a suitable bacterial cell. Suitable bacterial cells include but are not limited to bacterial cells from the bacteria of the species Escherichia coli. Many suitable strains of E. coli are known in the art for expression of vectors. These include, but are not limited to Pir1, Stbl2, Stbl3, Stbl4, TOP10, XL1 Blue, and XL10 Gold. In some embodiments, the host cell is a suitable insect cell. Suitable insect cells include those from Spodoptera frugiperda. Suitable strains of S. frugiperda cells include but are not limited to 519 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2O5, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990).

In some embodiments, the vector can be a yeast expression vector. Examples of vectors for expression in yeast Saccharomyces cerevisiae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 211 plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329: 840) and pMT2PC (Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, where one or more modified KTI polynucleotides, functional domains thereof, and/or one or more additional polynucleotides are included and/or expressed (e.g. a reporter polynucleotide), each be operably linked to separate regulatory elements on the same or separate vectors. In some embodiments, two or more of the elements expressed from the same or different regulatory element(s), can be combined in a single vector, with one or more additional vectors providing any components of the system not included in the first vector. Encoding polynucleotides (modified KTI genes or others) that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding one or more modified KTI proteins and/or functionals domains thereof and/or one or more other genes (e.g., reporter gene), embedded within one or more intron sequences (e.g., each in a different intron, two or more in at least one intron, or all in a single intron). In some embodiments, the modified KTI proteins and/or functionals domains thereof and/or one or more other genes (e.g., reporter gene) can be operably linked to and expressed from the same promoter.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

Where a vector or vector system is provided that contains the modified KTI encoding polynucleotide, the encoding polynucleotide can be operatively coupled to one or more regulatory elements. In some embodiments, the regulatory element can drive ubiquitous expression. In some embodiments, the regulatory element can drive or control cell or tissue specific expression. In some embodiments, the regulatory element can drive conditional or inducible expression. In some embodiments, the modified KTI encoding polynucleotide is operatively coupled to a plant promoter.

In embodiments, the polynucleotides and/or vectors thereof described herein (such as the modified KTI encoding polynucleotides) can include one or more regulatory elements that can be operatively linked to the polynucleotide. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as leaves, stem, fruit, flower, etc., or particular cell types. Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In some embodiments, the regulatory sequence can be a regulatory sequence described in U.S. Pat. No. 7,776,321, U.S. Pat. Pub. No. 2011/0027239, and PCT publication WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

To express a polynucleotide, the vector can include one or more transcriptional and/or translational initiation regulatory sequences, e.g., promoters, that direct the transcription of the gene and/or translation of the encoded protein in a cell.

Inducible/conditional promoters can be positively inducible/conditional promoters (e.g. a promoter that activates transcription of the polynucleotide upon appropriate interaction with an activated activator, or an inducer (compound, environmental condition, or other stimulus) or a negative/conditional inducible promoter (e.g., a promoter that is repressed (e.g., bound by a repressor) until the repressor condition of the promotor is removed (e.g., inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Where expression in a plant cell is desired, the modified KTI encoding polynucleotides described herein are typically placed under control of a plant promoter, i.e., a promoter operable in plant cells. The use of different types of promoters is envisaged.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters may direct the expression of a gene in different tissues or cell types, or at different stages of development, or in response to different environmental conditions. In particular embodiments, one or more of the modified KTI encoding polynucleotides are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed. Examples of particular promoters for use in expression of the modified KTI encoding polynucleotides are found in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

Further exemplary plant promoters suitable to drive expression of the heterologous modified KTI gene product encoding polynucleotides include those obtained from plants, plant viruses, and bacteria such as Agrobacterium or Rhizobium which comprise genes expressed in plant cells. Additional examples of promoters include those described in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

Examples of promoters that are inducible and that can allow for spatiotemporal control of gene editing or gene expression may use a form of energy. The form of energy may include, but is not limited to, sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome), such as a Light Inducible Transcriptional Effector (LITE) that direct changes in transcriptional activity in a sequence-specific manner. The components of a light inducible system may include one or more modified KTI encoding polynucleotides described herein, a light-responsive cytochrome heterodimer (e.g., from Arabidopsis thaliana), and a transcriptional activation/repression domain. In some embodiments, the vector can include one or more of the inducible DNA binding proteins provided in PCT publication WO 2014/018423 and US Publications, 2015/0291966, 2017/0166903, 2019/0203212, which describe e.g., embodiments of inducible DNA binding proteins and methods of use and can be adapted for use with the present invention.

In some embodiments, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-ll-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters that are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

Where transient expression of an encoding polynucleotide described herein is desired, transient expression may be achieved using suitable vectors. Exemplary vectors that may be used for transient expression include a pEAQ vector (may be tailored for Agrobacterium-mediated transient expression) and Cabbage Leaf Curl virus (CaLCuV), and vectors described in Sainsbury F. et al., Plant Biotechnol J. 2009 September; 7(7):682-93; and Yin K et al., Scientific Reports volume 5, Article number: 14926 (2015).

A plant promoter is capable of initiating transcription in plant cells, whether or not its origin is a plant cell. The use of different types of promoters is envisaged.

In some embodiments, the vector or system thereof can include one or more elements capable of translocating and/or expressing a modified KTI polypeptide encoding polynucleotide to/in a specific cell component or organelle. Such organelles can include, but are not limited to, nucleus, ribosome, endoplasmic reticulum, golgi apparatus, chloroplast, mitochondria, vacuole, lysosome, cytoskeleton, plasma membrane, cell wall, peroxisome, centrioles, etc.

### Selectable Markers and Tags

One or more of the modified KTI polypeptide encoding polynucleotides can be operably linked, fused to, or otherwise modified to include a polynucleotide that encodes or is a selectable marker or tag, which can be a polynucleotide or polypeptide. In some embodiments, the polynucleotide encoding a polypeptide selectable marker can be incorporated with the modified KTI polypeptide encoding polynucleotides polynucleotide such that the selectable marker polypeptide, when translated, is inserted between two amino acids between the N- and C-terminus of the modified KTI polypeptide or at the N- and/or C-terminus of the modified KTI polypeptide. In some embodiments, the selectable marker or tag is a polynucleotide barcode or unique molecular identifier (UMI).

It will be appreciated that the polynucleotide encoding such selectable markers or tags can be incorporated into a modified KTI polypeptide encoding polynucleotide described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more modified KTI polypeptides described herein via suitable linker, such as a glycine or glycine serine linkers which are known in the art.

The vector or vector system can include one or more polynucleotides encoding one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organelles, etc. In some embodiments, the targeting moiety encoding polynucleotides can be included in the vector or vector system such that the modified KTI polynucleotides and/or products expressed therefrom include the targeting moiety and can be targeted to specific cells, tissues, organelles, etc. In some embodiments, such as non-viral carriers, the targeting moiety can be attached to the carrier (e.g., polymer, lipid, inorganic molecule etc.) and can be capable of targeting the carrier and any attached or associated modified KTI polynucleotide(s) to specific cells, tissues, organelles, etc.

### Cell-Free Vector and Polynucleotide Expression

In some embodiments, the polynucleotide encoding a modified KTI gene product is expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg₂₊, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems transcription and translation are coupled and DNA is first transcribed into RNA, which is subsequently translated. Suitable standard and coupled cell-free translation systems are generally known in the art and are commercially available.

### Codon Optimization of Vector Polynucleotides

As described elsewhere herein, the modified KTI gene product encoding polynucleotides described herein can be codon optimized. In some embodiments, one or more polynucleotides contained in a vector (“vector polynucleotides”) described herein that are in addition to an optionally codon optimized the modified KTI gene product encoding polynucleotides described herein can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11.; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

The vector polynucleotide can be codon optimized for expression in a specific cell-type, tissue type, organ type, and/or subject type. In some embodiments, a codon optimized sequence is a sequence optimized for expression in a eukaryote, e.g., humans (i.e., being optimized for expression in a human or human cell), or for another eukaryote, such as another animal (e.g., a mammal or avian) as is described elsewhere herein. Such codon optimized sequences are within the ambit of the ordinary skilled artisan in view of the description herein. In some embodiments, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g., astrocytes, glial cells, Schwann cells etc.), muscle cells (e.g., cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Non-Viral Vectors and Carriers

In some embodiments, the vector is a non-viral vector or carrier. In some embodiments, non-viral vectors can have the advantage(s) of reduced toxicity and/or immunogenicity and/or increased bio-safety as compared to viral vectors The terms of art “Non-viral vectors and carriers” and as used herein in this context refers to molecules and/or compositions that are not based on one or more component of a virus or virus genome (excluding any nucleotide to be delivered and/or expressed by the non-viral vector) that can be capable of attaching to, incorporating, coupling, and/or otherwise interacting with a heterologous modified KTI polypeptide encoding polynucleotide and can be capable of ferrying the polynucleotide to a cell and/or expressing the polynucleotide. It will be appreciated that this does not exclude the inclusion of a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors and carriers include naked polynucleotides, chemical-based carriers, polynucleotide (non-viral) based vectors, and particle-based carriers. It will be appreciated that the term “vector” as used in the context of non-viral vectors and carriers refers to polynucleotide vectors and “carriers” used in this context refers to a non-nucleic acid, polynucleotide molecule, or composition that be attached to or otherwise interact with, encapsulate, and/or associate with a polynucleotide to be delivered, such as a modified KTI gene product encoding polynucleotide of the present invention.

### Naked Polynucleotides

In some embodiments, one or more modified KTI gene product encoding polynucleotides described elsewhere herein can be included in and/or delivered as a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the modified KTI gene product encoding polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In some embodiments, the naked polynucleotide contains only the modified KTI gene product encoding polynucleotide(s) described herein. In some embodiments, the naked polynucleotide can contain other nucleic acids and/or polynucleotides in addition to the modified KTI gene product encoding polynucleotide(s) described herein. The naked polynucleotides can include one or more elements of a transposon system. Transposons and system thereof are described in greater detail elsewhere herein.

### Non-Viral Polynucleotide Vectors

In some embodiments, one or more of the modified KTI gene product encoding polynucleotides can be included in a non-viral polynucleotide vector. Suitable non-viral polynucleotide vectors include, but are not limited to, transposon vectors and vector systems, plasmids, bacterial artificial chromosomes, yeast artificial chromosomes, AR (antibiotic resistance)-free plasmids and miniplasmids, circular covalently closed vectors (e.g. minicircles, minivectors, miniknots,), linear covalently closed vectors (“dumbbell shaped”), MIDGE (minimalistic immunologically defined gene expression) vectors, MiLV (micro-linear vector) vectors, Ministrings, mini-intronic plasmids, agrobacterium vectors (Ti or Ri vectors), PSK systems (post-segregationally killing systems), ORT (operator repressor titration) plasmids, and the like. See e.g., Hardee et al. 2017. Genes. 8(2):65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In embodiments, the S/MAR sequence is located downstream of an actively transcribed polynucleotide (e.g., one or more modified KTI gene product encoding polynucleotides described herein) included in the non-viral polynucleotide vector. In some embodiments, the S/MAR can be a S/MAR from the beta-interferon gene cluster. See e.g., Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these embodiments, the transposon vector lacks one or more polynucleotide sequences encoding proteins required for transposition. In some embodiments, the non-autonomous transposon vectors lack one or more Ac elements.

In some embodiments a non-viral polynucleotide transposon vector system can include a first polynucleotide vector that contains the modified KTI gene product encoding polynucleotide(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g., the modified KTI gene product encoding polynucleotide(s)) and integrate it into one or more positions in the host cell's genome. In some embodiments, the transposon vector or system thereof can be configured as a gene trap. In some embodiments, the TIRs can be configured to flank a strong splice acceptor site followed by a reporter and/or other gene (e.g., one or more of the modified KTI gene product encoding polynucleotide(s)) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include, without limitation, Sleeping Beauty transposon system (Tc1/mariner superfamily) (see e.g., Ivies et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tc1/mariner superfamily) (see e.g., Miskey et al. 2003 Nucleic Acid Res. 31(23):6873-6881) and variants thereof.

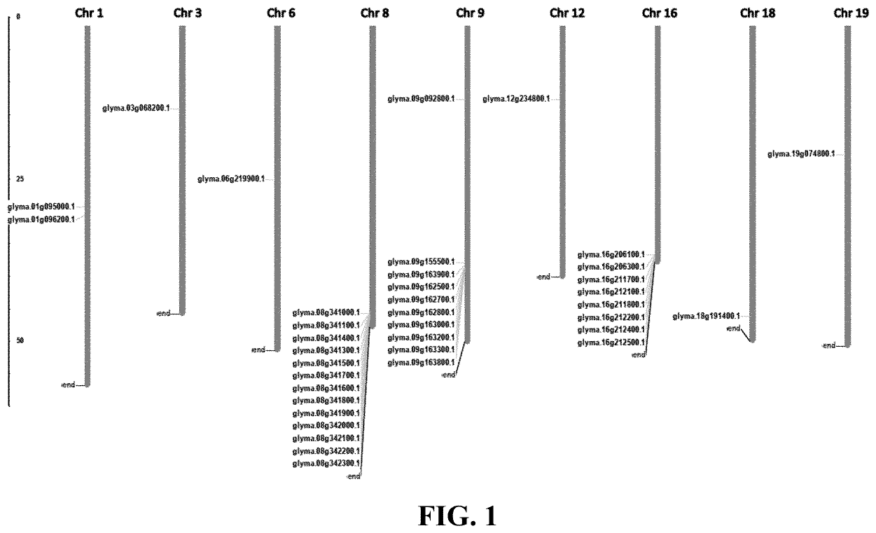

In some embodiments, the non-viral vector or vector system is an agrobacterium vector or vector system In some embodiments, the modified KTI gene product encoding polynucleotide(s) is included in a T-DNA (or Ti) vector or an Ri vector (See e.g., Gelvin, S. 2003. Microbiol Mol Biol Rev. 2003 March; 67(1): 16-37, particularly at FIG. 1A; Lee and Gelvin. Plant Physiol. 2008 February; 146(2): 325-332 and as described elsewhere herein.

### Chemical Carriers

In some embodiments, the modified KTI gene product encoding polynucleotide(s) can be coupled to a chemical carrier. Chemical carriers that can be suitable for delivery of polynucleotides can be broadly classified into the following classes: (i) inorganic particles, (ii) lipid-based, (iii) polymer-based, and (iv) peptide based. They can be categorized as (1) those that can form condensed complexes with a polynucleotide (such as the modified KTI gene product encoding polynucleotide(s)), (2) those capable of targeting specific cells, (3) those capable of increasing delivery of the polynucleotide (such as the modified KTI gene product encoding polynucleotide(s)) to the nucleus or cytosol of a host cell, (4) those capable of disintegrating from DNA/RNA in the cytosol of a host cell, and (5) those capable of sustained or controlled release. It will be appreciated that any one given chemical carrier can include features from multiple categories. The term “particle” as used herein, refers to any suitable sized particles for delivery of the modified KTI gene product encoding polynucleotide(s) described herein. Suitable sizes include macro-, micro-, and nano-sized particles.

In some embodiments, the non-viral carrier can be an inorganic particle. In some embodiments, the inorganic particle, can be a nanoparticle. The inorganic particles can be configured and optimized by varying size, shape, and/or porosity. In some embodiments, the inorganic particles are optimized to escape from the reticuloendothelial system. In some embodiments, the inorganic particles can be optimized to protect an entrapped molecule from degradation. The suitable inorganic particles that can be used as non-viral carriers in this context can include, but are not limited to, calcium phosphate, silica, metals (e.g. gold, platinum, silver, palladium, rhodium, osmium, iridium, ruthenium, mercury, copper, rhenium, titanium, niobium, tantalum, and combinations thereof), magnetic compounds, particles, and materials, (e.g. supermagnetic iron oxide and magnetite), quantum dots, fullerenes (e.g. carbon nanoparticles, nanotubes, nanostrings, and the like), and combinations thereof. Other suitable inorganic non-viral carriers are discussed elsewhere herein.

In some embodiments, the non-viral carrier can be lipid-based. Suitable lipid-based carriers are also described in greater detail herein. In some embodiments, the lipid-based carrier includes a cationic lipid or an amphiphilic lipid that is capable of binding or otherwise interacting with a negative charge on the polynucleotide to be delivered (e.g., such as a modified KTI gene product encoding polynucleotide). In some embodiments, chemical non-viral carrier systems can include a polynucleotide such as the modified KTI gene product encoding polynucleotide (s)) and a lipid (such as a cationic lipid). These are also referred to in the art as lipoplexes. Other embodiments of lipoplexes are described elsewhere herein. In some embodiments, the non-viral lipid-based carrier can be a lipid nano emulsion. Lipid nano emulsions can be formed by the dispersion of an immiscible liquid in another stabilized emulsifying agent and can have particles of about 200 nm that are composed of the lipid, water, and surfactant that can contain the polynucleotide to be delivered (e.g., the modified KTI gene product encoding polynucleotide(s)). In some embodiments, the lipid-based non-viral carrier can be a solid lipid particle or nanoparticle.

In some embodiments, the non-viral carrier can be peptide-based. In some embodiments, the peptide-based non-viral carrier can include one or more cationic amino acids. In some embodiments, 35 to 40, 45, 50, 55, 60, 65, 70, 75, 80, 85, 90, 95, 99 or 100% of the amino acids are cationic. In some embodiments, peptide carriers can be used in conjunction with other types of carriers (e.g., polymer-based carriers and lipid-based carriers to functionalize these carriers). In some embodiments, the functionalization is targeting a host cell. Suitable polymers that can be included in the polymer-based non-viral carrier can include, but are not limited to, polyethylenimine (PEI), chitosan, poly (DL-lactide) (PLA), poly (DL-Lactide-co-glycoside) (PLGA), dendrimers (see e.g., US Pat. Pub. 2017/0079916 whose techniques and compositions can be adapted for use with the modified KTI gene product encoding polynucleotide(s)), polymethacrylate, and combinations thereof.

In some embodiments, the non-viral carrier can be configured to release an engineered delivery system polynucleotide that is associated with or attached to the non-viral carrier in response to an external stimulus, such as pH, temperature, osmolarity, concentration of a specific molecule or composition (e.g., calcium, NaCl, and the like), pressure and the like. In some embodiments, the non-viral carrier can be a particle that is configured includes one or more of the modified KTI gene product encoding polynucleotide(s) described herein and an environmental triggering agent response element, and optionally a triggering agent. In some embodiments, the particle can include a polymer that can be selected from the group of polymethacrylates and polyacrylates. In some embodiments, the non-viral particle can include one or more embodiments of the compositions microparticles described in US Pat. Pubs. 20150232883 and 20050123596, whose techniques and compositions can be adapted for use in the present invention.

In some embodiments, the non-viral carrier can be a polymer-based carrier. In some embodiments, the polymer is cationic or is predominantly cationic such that it can interact in a charge-dependent manner with the negatively charged polynucleotide to be delivered (such as the modified KTI gene product encoding polynucleotide(s)).

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as a modified KTI gene product encoding polynucleotide, into a virus particle and producing said virus particle when used alone or with one or more other viral vectors (such as in a viral vector system). Viral vectors and systems thereof can be used for producing viral particles for delivery of and/or expression of one or more components of the modified KTI gene product encoding polynucleotide described herein. The viral vector can be part of a viral vector system involving multiple vectors. In some embodiments, systems incorporating multiple viral vectors can increase the safety of these systems. Suitable viral vectors can include any plant viral vector or system such as any of those set forth in e.g., K. Hefferon. Biomedicines. Zaidi* and Mansoor. 2017 September; 5(3): 44, Front. Plant Sci., 11 Apr. 2017. https://doi.org/10.3389/fpls.2017.00539, and Abrahamian et al. 2020. Ann. Rev. Virol. 7:513-535, particularly those based on tobacco mosaic virus (TMV), Potexviruses, and Comovirus Cowpea mosaic virus (CPMV). Other embodiments of viral vectors and viral particles produce therefrom are described elsewhere herein. In some embodiments, the viral vectors are configured to produce replication incompetent viral particles for improved safety of these systems.

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Application publication No. US 2004-0171156 A1. Other suitable methods and techniques are described elsewhere herein.

In some embodiments, the vector can have one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

Virus Particle Production from Viral Vectors

In some embodiments, one or more viral vectors and/or system thereof can be delivered to a suitable cell line for production of virus particles containing the polynucleotide or other payload to be delivered to a host cell. Suitable host cells for virus production from viral vectors and systems thereof described herein are known in the art and are commercially available. For example, suitable host cells include HEK 293 cells and its variants (HEK 293T and HEK 293TN cells). In some embodiments, the suitable host cell for virus production from viral vectors and systems thereof described herein can stably express one or more genes involved in packaging (e.g., pol, gag, and/or VSV-G) and/or other supporting genes.

In some embodiments, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g., a modified KTI gene product encoding polynucleotide), and virus particle assembly, and secretion of mature virus particles into the culture media. Various other methods and techniques are generally known to those of ordinary skill in the art.

Mature virus particles can be collected from the culture media by a suitable method. In some embodiments, this can involve centrifugation to concentrate the virus. The titer of the composition containing the collected virus particles can be obtained using a suitable method. Such methods can include transducing a suitable cell line (e.g., NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In some embodiments, the resulting composition containing virus particles can contain 1×10¹-1×10²⁰ particles/mL.

### Vector and Virus Particle Delivery

A vector (including non-viral carriers) described herein can be introduced into host cells to thereby produce transcripts, proteins, or peptides, including fusion proteins or peptides encoded by nucleic acids as described herein (e.g., modified KTI transcripts, proteins, enzymes, mutant forms thereof, fusion proteins thereof, etc.), and virus particles (such as from viral vectors and systems thereof).

One or more modified KTI gene product encoding polynucleotides can be delivered using an engineered plant virus particle containing the one or more modified KTI gene product encoding polynucleotides using appropriate formulations and doses.

The vector(s) and virus particles described herein can be delivered into a host cell in vitro, in vivo, and or ex vivo. Delivery can occur by any suitable method including, but not limited to, physical methods, chemical methods, and biological methods. Physical delivery methods are those methods that employ physical force to counteract the membrane barrier of the cells to facilitate intracellular delivery of the vector. Suitable physical methods include, but are not limited to, needles (e.g., injections), ballistic polynucleotides (e.g., particle bombardment, micro projectile gene transfer, and gene gun), electroporation, sonoporation, photoporation, magnetofection, hydroporation, and mechanical massage. Chemical methods are those methods that employ a chemical to elicit a change in the cells membrane permeability or other characteristic(s) to facilitate entry of the vector into the cell. For example, the environmental pH can be altered which can elicit a change in the permeability of the cell membrane. Biological methods are those that rely and capitalize on the host cell's biological processes or biological characteristics to facilitate transport of the vector (with or without a carrier) into a cell. For example, the vector and/or its carrier can stimulate an endocytosis or similar process in the cell to facilitate uptake of the vector into the cell.

### Methods and Techniques for Generating the Engineered Plants

Methods and techniques for introducing mutations, insertions, deletions, substitutions, or any combination thereof are generally known in the art and described elsewhere herein. Such methods and techniques include, but are not limited to, recombinant engineering and genome editing via programmable nucleases (e.g., TALENs, Zinc Finger nuclease, CRISPR-Cas systems, CRISPR-Cas based systems (e.g., CRISPR-associated transposases (CAST), and Primer Editors, and/or the like). See e.g., Malzahn et al., Cell Biosci. 2017 Apr. 24; Molla et al., Nat Plants. 2021. PMID: 34518669k 7:21. doi: 10.1186/s13578-017-0148-4. eCollection 2017; Zhang et al., Prog Mol Biol Transl Sci. 2017; 149:133-150. doi: 10.1016/bs.pmbts.2017.03.008; Sprink et al., Curr Opin Biotechnol. 2015 April; 32:47-53. doi: 10.1016/j.copbio.2014.11.010; Razzaq et al., Int J Mol Sci. 2019 Aug. 19; 20(16):4045. doi: 10.3390/ijms20164045; Sanagala et al., Sanagala R, et al. J Genet Eng Biotechnol. 2017. PMID: 306476; Norman et al., Front Plant Sci. 2016. PMID: 27917188; Molka et al., Strecker et al., Science. 2019. 365(6448):48-53; and Nandy et al., J Biosci. 2020. PMID: 32020912, which can be adapted for use with the present disclosure. In view of at least the reference sequences in the context of the disclosure herein, one of ordinary skill in the art can apply any one or more of these methods and techniques to generate engineered plants, such as soybean plants, having one or more modified KTI genes and/or gene products. In some embodiments, such as when the engineered plant is generated using a CRISPR-Cas system or CRISPR-based system, the engineered plant comprises a CRISPR-Cas system or component(s) thereof. In some embodiments, such as when the engineered plant is generated using a CRISPR-Cas system or CRISPR-based system, the engineered plant does not comprise a CRISPR-Cas system or component(s) thereof. For example, the CRISPR-Cas components can be delivered such that their expression and/or presence in the plant cell is temporary.

### Engineered Plants Having Modified KTI Gene Products

As previously described in some embodiments, the engineered plant has one or more modified KTI gene products as compared to a suitable control. In some embodiments, the one or more modified KTI gene products is mRNA, a polypeptide, or both. In some embodiments, the modified KTI gene product is modified as compared to a suitable control. In some embodiments, the suitable control is the unmodified KTI gene product.

In some embodiments, the modified KTI gene product is (a) a modified KTI mRNA, wherein the mRNA has been modified as compared to an unmodified control to decrease protein translation, mRNA stability, or both; (b) a modified KTI polypeptide, wherein the polypeptide has been modified as compared to an unmodified control to decrease protein stability; or both (a) and (b).

mRNA Modifications

In some embodiments, the modified KTI mRNA comprises (a) one or more mutations, insertions, deletions, substitutions, or any combination thereof such that translation and/or stability of the KTI mRNA is increased; (b) one or more nucleic acid modifications that increases translation and/or mRNA stability; or both (a) and (b).

In some embodiments, the modifications are genetically encoded in DNA that encodes the mRNA such that when transcribed, the one or more modifications are present in the transcribed mRNA so as to produce the modified mRNA. In other embodiments, the mRNA can be directly modified, such as by a programmable nuclease. Methods of using programmable nuclease systems, such as CRISPR-Cas based systems, and other systems to modify RNA are generally known in the art. See e.g., Porto et al., Nat Rev Drug Discov. 2020 December; 19(12):839-859. doi: 10.1038/s41573-020-0084-6; Rees et al., Nat Rev Genet. 2018 December; 19(12):770-788. doi: 10.1038/s41576-018-0059-1; Xu et al., Mol Cell. 2022 Jan. 20; 82(2):389-403. doi: 10.1016/j.molcel.2021.10.010; Li et al., J Zhejiang Univ Sci B. 2021 Apr. 15; 22(4):253-284. doi: 10.1631/jzus.B2100009; Zeballos and Gaj. Trends Biotechnol. 2021 July; 39(7):692-705. doi: 10.1016/j.tibtech.2020.10.010; Lo et al., Front Genet. 2022 Jan. 28; 13:834413. doi: 10.3389/fgene.2022.834413. eCollection 2022; Li et al., Funct Integr Genomics. 2022 December; 22(6):1089-1103. doi: 10.1007/s10142-022-00910-3; Khosravi and Jantsch. RNA Biol. 2021 Oct. 15; 18(sup1):41-50. doi: 10.1080/15476286.2021.1983288; Kavuri et al., Cells. 2022 Aug. 27; 11(17):2665. doi: 10.3390/cells11172665, which can be adapted for use with the present disclosure, particularly to generate the engineered plants having modified KTI mRNA.

In some embodiments, an insertion, deletion, or indel in a modified endogenous KTI (e.g., KTI1 and/or KTI3) gene can range in size from 1-50 or more base pairs. In some embodiments, an insertion, deletion, or indel in a modified endogenous KTI gene can be 1 to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 base pairs or more.

In some embodiments, the modified KTI RNA has 1-500 or more mutated or substituted bases or base pairs In some embodiments, the modified KTI RNA has 1 to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 or more mutated or substituted bases or base pairs.

In some embodiments, the KTI RNA, such as mRNA contains one or more modifications that can modulate stability of the KTI RNA. Without being bound by theory some embodiments, where the modification increases stability the translation of a KTI RNA, such as an mRNA, is increased, which can increase the amount of a KTI polypeptide. Without being bound by theory some embodiments, where the modification decreases stability the translation of a KTI RNA, such as an mRNA, is decreased, which can decrease the amount of a KTI polypeptide. In some embodiments, the modifications that modulate the stability of the KTI RNA are genetically encoded or otherwise introduced into the sequence of the mRNA. In some embodiments, the modifications that modulate the stability of the KTI RNA are chemical modifications or synthetic bases that can be coupled to or otherwise integrated into the KTI RNA.

In some embodiments, the polynucleotides of a KTI RNA are structurally modified and/or chemically modified. As used in this context herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to affect a structural modification, structural modifications are of a chemical nature and hence are chemical modifications. However, structural modifications will result in a different sequence of nucleotides. For example, the polynucleotide “ATCG” may be chemically modified to “AT-SmeC-G”. The same polynucleotide may be structurally modified from “ATCG” to “ATCCCG”. Here, the dinucleotide “CC” has been inserted, resulting in a structural modification to the polynucleotide.

In some embodiments, the KTI RNA polynucleotide, e.g., an mRNA, described herein comprises at least one chemical modification. In some embodiments, the at least one chemical modification is selected from pseudouridine, N1-methylpseudouridine, N1-ethylpseudouridine, 2-thiouridine, 4′-thiouridine, 5-methyl cytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, the chemical modification is in the 5-position of the uracil. In some embodiments, the chemical modification is a N1-methylpseudouridine. In some embodiments, the chemical modification is a N1-ethylpseudouridine.

In some embodiments, about 10%, 15%, 20%, 24%, 30%, 35%, 40%, 45%, 50%, 55%, 60%, 65%, 70%, 75%, 80%, 85%, 90%, 95%, to/or about 100% of the uracil in of the KTI RNA, such in the open reading frame of the KTI encoding polynucleotide, have a chemical modification.

In some embodiments, the KTI mRNA polynucleotide includes a stabilization element. In some embodiments, the stabilization element is a histone stem-loop. In some embodiments, the stabilization element is a nucleic acid sequence having increased GC content relative to wild type sequence.

In some embodiments, the KTI mRNA comprises and/or encodes one or more 5′ terminal cap (or cap structure), 3′ terminal cap, 5′untranslated region, 3′untranslated region, a tailing region, or any combination thereof. In some embodiments, the capping region of the KTI mRNA region may be from 1 to 10, e.g., 2-9, 3-8, 4-7, 1-5, 5-10, or at least 2, or 10 or fewer nucleotides in length. In some embodiments, the cap is absent. In some embodiments, a 5′-cap structure is cap0, cap1, ARCA, inosine, N1-methyl-guanosine, 2′-fluoro-guanosine, 7-deaza-guanosine, 8-oxo-guanosine, 2-amino-guanosine, LNA-guanosine, or 2-azido-guanosine. In some embodiments, the 5′terminal cap is 7mG(5′)ppp(5′)NlmpNp, m7GpppG cap, N⁷-methylguanine. In some embodiments, the 3′terminal cap is a 3′-O-methyl-m7GpppG.

In some embodiments, the 3′-UTR is an alpha-globin 3′-UTR. In some embodiments, the 5′-UTR comprises a Kozak sequence.

In some embodiments, the tailing sequence may range from absent to 500 nucleotides in length (e.g., at least 60, 70, 80, 90, 120, 140, 160, 180, 200, 250, 300, 350, 400, 450, or 500 nucleotides). In some embodiments, the tailing region is or includes a polyA tail. Where the tailing region is a polyA tail, the length may be determined in units of or as a function of polyA Binding Protein binding. In this embodiment, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional. In some embodiments, the poly-A tail is at least 160 nucleotides in length.

Other RNA modifications for mRNAs and production of mRNA can be as described e.g., U.S. Pat. Nos. 8,278,036, 8,691,966, 8,748,089, 9,750,824, 10,232,055, 10,703,789, 10,702,600, 10,577,403, 10,442,756, 10,266,485, 10,064,959, 9,868,692, 10,064,959, 10,272,150; U.S. Publications, US20130197068, US20170043037, US20130261172, US20200030460, US20150038558, US20190274968, US20180303925, US20200276300; International Patent Application Publication Nos. WO/2018/081638A1, WO/2016/176330A1, which are incorporated herein by reference and can be adapted for use with the present invention.

### Polypeptide Modifications

In some embodiments, the one or more modified gene products is one or more modified KTI (e.g., KTI1 and/or KTI3) polypeptide. In some embodiments, at least one of the one or more modified KTI polypeptides has decreased activity as compared to an unmodified control. In some embodiments, the engineered plant having decreased KTI (e.g., KTI1 and/or KTI3) enzyme activity has one or more modified KTI polypeptides. In some embodiments, one or more modified KTI polypeptides comprises one or more amino acid mutations, insertions, deletions, substitutions, or any combination thereof such that activity of the one or more modified KTI polypeptides is increased as compared to a control; (b) comprises one or more post-translational modifications such that activity of the one or more modified KTI polypeptides is increased as compared to a control.

The modified KTI polypeptide can by a KTI1 or KTI3 that contains 1 or more mutations, insertions, deletions, indels, substitutions, or any combination thereof of one or more continuous or discontinuous amino acids. In some embodiments, the modified KTI polypeptide is encoded by a sequence 80-100% identical to any one of SEQ ID NO: 5-9.

In some embodiments, the modification is a post-translational modification. The post translational modification can be reversible or irreversible. The post translational modifications can modulate one or more activities or functions of the KTI polypeptide, such as protein lifespan, protein-protein interactions, cell to cell and/or cell to cell-matrix interactions, molecular trafficking, receptor activation and/or ligand binding, protein solubility, protein folding, protein localization and/or any combination thereof. Exemplary post translational modifications include, but are not limited to glycosylation, methylation, phosphorylation, acetylation, succinylation, malonylation, sumolation, S-nitrosylation, glutathionylation, amidation, hydroxylation, palmitolation, pyrrolidone carboxcylic acid, glutarylation, gamma-carboxyglutamic acid, crotonylation, oxidation, myristoylation, sulfantion, formylation, and citrullination. See e.g., Ramazi and Zahiri. Database, Volume 2021, 2021, baab012, https://doi.org/10.1093/database/baab012 for additional exemplary post translational modifications. In some embodiments, the amino acid sequence of a KTI polypeptide is modified to influence the post-translational modification. For example, in some embodiments, the KTI polypeptide is modified so as to increase or decrease the number of residues capable of being post translationally modified by any one or more post translational modifications.

In some embodiments, the KTI polypeptide contains one or more signaling and/or trafficking polypeptides. Exemplary signaling and trafficking sequences can be genetically encoded and are described in greater detail elsewhere herein. In some embodiments, the KTI polypeptide contains or is otherwise operatively coupled to one or more reporter polypeptides and/or polynucleotides. Exemplary reporter polypeptides and/or polynucleotides are described in greater detail elsewhere herein.

In some embodiments, the engineered cell, the engineered plant, or both include a modified KTI gene and/or modified KTI gene product encoding polynucleotide stably integrated into the genome of the engineered cell.

In some embodiments, the engineered cell, the engineered plant, or both include a modified KTI gene and/or modified KTI gene product encoding polynucleotide that is transiently expressed in the engineered cell, engineered plant, or both.

Methods of Generating Engineered Soybean Plants having Modified KTI Genes and/or Gene Products

Also described herein are methods of modifying a plant cell such that it contains and optionally expresses a modified KTI polypeptide and/or modified KTI gene and/or modified KTI gene product encoding polynucleotide and/or vector or vector system containing a modified KTI gene product encoding polynucleotide, or a combination thereof. In some embodiments, the method includes modifying a plant cell such that the plant cell contains and optionally expresses a heterologous or non-native modified KTI polypeptide, a vector or vector system containing a non-native or heterologous modified KTI gene or modified KTI gene product encoding polynucleotide, or a combination thereof. In some embodiments, modifying includes delivering a polynucleotide having a sequence that is about 80-100% (e.g., 80 to/or 81, 82, 83, 84, 85, 86, 87, 88, 89, 90, 91, 92, 93, 94, 95, 96, 97, 98, 99, 100%) identical to (a) any one or more of SEQ ID NO: 6-9 or 49, (b) any one of SEQ ID NO: 5, or (c) both (a) and (b) or a vector or vector system thereof to the cell.

In some embodiments, the modified plants or plant cells may be cultured to regenerate a whole plant which possesses the transformed or modified genotype and thus the desired phenotype. Examples of regeneration techniques include those relying on manipulation of certain phytohormones in a tissue culture growth medium, relying on a biocide and/or herbicide marker which has been introduced together with the desired nucleotide sequences, obtaining from cultured protoplasts, plant callus, explants, organs, pollens, embryos or parts thereof.

“Plant” as used herein encompasses any plant tissue or part of the plant of the invention. In some embodiments, said part is selected from the group of a plant cell, a somatic embryo, a pollen, a gametophyte, an ovule, an inflorescence, a leaf, a seedling, a stem, a callus, a stolon, a microtuber, a root, a shoot, a seed, a fruit and a spore. Further encompassed are T1 generation plants produced from the seeds of the transformed plant (TO). Any suitable method can be used to confirm and detect the modification made in the plant. Such methods are generally known in the art. In some examples, when a variety of modifications are made, one or more desired modifications or traits resulting from the modifications may be selected and detected. The detection and confirmation may be performed by biochemical and molecular biology techniques such as Southern analysis, PCR, Northern blot, Si RNase protection, primer-extension or reverse transcriptase-PCR, enzymatic assays, ribozyme activity, gel electrophoresis, Western blot, immunoprecipitation, enzyme-linked immunoassays, in situ hybridization, enzyme staining, and immunostaining.

A part of a plant, e.g., a “plant tissue” can be engineered to include a modified KTI (e.g., KTI1 and/or KTI3) gene product encoding polynucleotide, vector, and/or polypeptide described elsewhere herein to produce an improved plant. Plant tissue also encompasses plant cells. The term “plant cell” as used herein refers to individual units of a living plant, either in an intact whole plant or in an isolated form grown in in vitro tissue cultures, on media or agar, in suspension in a growth media or buffer or as a part of higher organized unites, such as, for example, plant tissue, a plant organ, or a whole plant.

A “protoplast” refers to a plant cell that has had its protective cell wall completely or partially removed using, for example, mechanical or enzymatic means resulting in an intact biochemical competent unit of living plant that can reform their cell wall, proliferate and regenerate grow into a whole plant under proper growing conditions.

In some cases, one or more markers, such as selectable and detectable markers, may be introduced to the plants. Such markers may be used for selecting, monitoring, isolating cells and plants with desired modifications and traits. A selectable marker can confer positive or negative selection and is conditional or non-conditional on the presence of external substrates. Examples of such markers include genes and proteins that confer resistance to antibiotics, such as hygromycin (hpt) and kanamycin (nptII), and genes that confer resistance to herbicides, such as phosphinothricin (bar) and chlorosulfuron (als), enzyme capable of producing or processing colored substances (e.g., the β-glucuronidase, luciferase, B or Cl genes).

Any suitable method may be used to deliver the transgene to the plant, plant cell, and/or plant cell population. Such transformation techniques are generally known in the art. Example methods and techniques include those in U.S. Pat. No. 6,603,061 —Agrobacterium-Mediated Plant Transformation Method; U.S. Pat. No. 7,868,149—Plant Genome Sequences and Uses Thereof and US 2009/0100536—Transgenic Plants with Enhanced Agronomic Traits, Morrell et al “Crop genomics: advances and applications,” Nat Rev Genet. 2011 Dec. 29; 13(2):85-96, all the contents and disclosure of each of which are herein incorporated by reference in their entirety.

In some embodiments, where transient expression is desired transgene DNA and/or RNA (e.g., mRNA) may be introduced to plant cells for transient expression. In such cases, the introduced nucleic acid may be provided in sufficient quantity to modify the cell but do not persist after a contemplated period of time has passed or after one or more cell divisions.

A method of generating engineered cells and/or plants can include transformation of one or more cells. The term “transformation” broadly refers to the process by which a plant host is genetically modified by the introduction of DNA by means of Agrobacteria or one of a variety of chemical or physical methods. As used herein, the term “plant host” refers to plants, including any cells, tissues, organs, or progeny of the plants. Many suitable plant tissues or plant cells can be transformed and include, but are not limited to, protoplasts, somatic embryos, pollen, leaves, seedlings, stems, calli, stolons, microtubers, roots, and shoots. A plant tissue also refers to any clone of such a plant, seed, progeny, propagule whether generated sexually or asexually, and descendants of any of these, such as cuttings or seed. The term “transformed” as used herein, refers to a cell, tissue, organ, or organism into which a foreign DNA molecule, such as a construct, has been introduced. The introduced DNA molecule may be integrated into the genomic DNA of the recipient cell, tissue, organ, or organism such that the introduced DNA molecule is transmitted to the subsequent progeny. In these embodiments, the “transformed” or “transgenic” cell or plant may also include progeny of the cell or plant and progeny produced from a breeding program employing such a transformed plant as a parent in a cross and exhibiting an altered phenotype resulting from the presence of the introduced DNA molecule. Preferably, the transgenic plant is fertile and capable of transmitting the introduced DNA to progeny through sexual reproduction.

In some embodiments, polynucleotides encoding the components of the compositions and systems may be introduced for stable integration into the genome of a plant cell. In some cases, vectors or expression systems may be used for such integration. The design of the vector or the expression system can be adjusted depending on for when, where and under what conditions the modified KTI gene and/or gene product system is expressed. Vectors and vector systems are described in greater detail elsewhere herein.

The term plant also encompasses progeny of the plant. The term “progeny”, such as the progeny of a transgenic (or engineered) plant, is one that is born of, begotten by, or derived from a plant or the transgenic plant. The introduced DNA molecule may also be transiently introduced into the recipient cell such that the introduced DNA molecule is not inherited by subsequent progeny and thus not considered “transgenic”. Accordingly, as used herein, a “non-transgenic” plant or plant cell is a plant which does not contain a foreign DNA stably integrated into its genome.

Also described herein are gametes, seeds, germplasm, embryos, either zygotic or somatic, progeny or hybrids of plants comprising the genetic modification (e.g., inclusion and/or expression of a modified KTI gene product encoding polynucleotide or modified KTI polynucleotide), which are produced by traditional breeding methods, are also included within the scope of the present invention. Such plants may contain a heterologous or foreign DNA sequence inserted at or instead of a target sequence. Alternatively, such plants may contain only an alteration (mutation, deletion, insertion, substitution) in one or more nucleotides. As such, such plants will only be different from their progenitor plants by the presence of the particular modification.

### Stable Integration in the Genome of Plants and Plant Cells

In particular embodiments, the polynucleotides encoding a modified KTI gene product are introduced for stable integration into the genome of a plant cell. In these embodiments, the design of the transformation vector or the expression system can be adjusted depending on for when, where and under what conditions the modified KTI gene product encoding polynucleotides are expressed. Suitable vectors and delivery are described in greater detail elsewhere herein.

In particular embodiments, the modified KTI gene product encoding polynucleotides are stably introduced into the genomic DNA of a plant cell. In particular embodiments, the modified KTI gene product encoding polynucleotides are introduced for stable integration into the DNA of a plant organelle such as, but not limited to a plastid, mitochondrion or a chloroplast. In some embodiments, the expression system for stable integration into the genome of a plant cell can contain one or more of the following elements: a promoter element that can be used to express modified KTI gene product encoding polynucleotide(s) in a plant cell; a 5′ untranslated region to enhance expression; an intron element to further enhance expression in certain cells, such as monocot cells; a multiple-cloning site to provide convenient restriction sites for inserting the polynucleotide modifying agent(s) or a system thereof and other desired elements; and a 3′ untranslated region to provide for efficient termination of the expressed transcript. The elements of the expression system may be on one or more expression constructs which are either circular such as a plasmid or transformation vector, or non-circular such as linear double stranded DNA.

DNA construct(s) containing the components of the systems, and, where applicable, template sequence may be introduced into the genome of a plant, plant part, or plant cell by a variety of conventional techniques. The process generally comprises the steps of selecting a suitable host cell or host tissue, introducing the construct(s) into the host cell or host tissue.

In particular embodiments, the DNA construct may be introduced into the plant cell using techniques such as but not limited to electroporation, microinjection, aerosol beam injection of plant cell protoplasts, or the DNA constructs can be introduced directly to plant tissue using biolistic methods, such as DNA particle bombardment (see also Fu et al., Transgenic Res. 2000 February; 9(1):11-9). The basis of particle bombardment is the acceleration of particles coated with gene/s of interest toward cells, resulting in the penetration of the protoplasm by the particles and typically stable integration into the genome. (see e.g., Klein et al, Nature (1987), Klein et ah, Bio/Technology (1992), Casas et ah, Proc. Natl. Acad. Sci. USA (1993).).

In particular embodiments, the DNA constructs containing components of the systems may be introduced into the plant by Agrobacterium-mediated transformation. The DNA constructs may be combined with suitable T-DNA flanking regions and introduced into a conventional Agrobacterium tumefaciens host vector. The foreign DNA can be incorporated into the genome of plants by infecting the plants or by incubating plant protoplasts with Agrobacterium bacteria, containing one or more Ti (tumor-inducing) plasmids. (see e.g., Fraley et al., (1985), Rogers et al., (1987) and U.S. Pat. No. 5,563,055).

### Transient Expression of in Plants and Plant Cells

In some embodiments, the modified KTI gene product encoding polynucleotides can be transiently expressed in the plant cell. In these embodiments, the system can ensure modified KTI gene product expression and TI inhibition can further be controlled. As the expression of the necessary components of the modified KTI gene product encoding polynucleotide(s) is transient, plants regenerated from such plant cells typically contain no foreign DNA.

In particular embodiments, the modified KTI gene product encoding polynucleotides can be transiently introduced in the plant cells using a plant viral vector (Scholthof et al. 1996, Annu Rev Phytopathol. 1996; 34:299-323). In further particular embodiments, said viral vector is a vector from a DNA virus. For example, geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). In other particular embodiments, said viral vector is a vector from an RNA virus. For example, tobravirus (e.g., tobacco rattle virus, tobacco mosaic virus), potexvirus (e.g., potato virus X), or hordeivirus (e.g., barley stripe mosaic virus). The replicating genomes of plant viruses are non-integrative vectors. Other suitable vectors are described elsewhere herein.

In particular embodiments, the vector used for transient expression of constructs in plants is for instance a pEAQ vector, which is tailored for Agrobacterium-mediated transient expression (Sainsbury F. et al., Plant Biotechnol J. 2009 September; 7(7):682-93) in the protoplast. Precise targeting of genomic locations was demonstrated using a modified Cabbage Leaf Curl virus (CaLCuV) vector to express gRNAs in stable transgenic plants expressing a CRISPR enzyme (Scientific Reports 5, Article number: 14926 (2015), doi:10.1038/srep14926).

In particular embodiments, double-stranded DNA fragments encoding a modified KTI gene product can be transiently introduced into the plant cell. In such embodiments, the introduced double-stranded DNA fragments are provided in sufficient quantity to modify the cell but do not persist after a contemplated period of time has passed or after one or more cell divisions. Methods for direct DNA transfer in plants are known by the skilled artisan (see for instance Davey et al. Plant Mol Biol. 1989 September; 13(3):273-85.)

In other embodiments, an RNA polynucleotide encoding the modified KTI gene product is introduced into the plant cell, which is then translated and processed by the host cell generating the protein in sufficient quantity to reduce TI, but which does not persist after a contemplated period of time has passed or after one or more cell divisions. Methods for introducing mRNA to plant protoplasts for transient expression are known by the skilled artisan (see for instance in Gallie, Plant Cell Reports (1993), 13; 119-122).

In some embodiments, a combination of the different methods described above can be used.

Translocation to and/or Expression in Specific Plant Organelles

The system may comprise elements for translocation to and/or expression in a specific plant organelle. In some embodiments, a tissue specific promoter can be included in the expression construct. In some embodiments, a tissue localization or organelle localization sequence or signal can be incorporated into the expression constructs. Such promoters and localization signals are described in greater detail elsewhere herein and/or will be appreciated by one of ordinary skill in the art. In some embodiments, the expression of the modified KTI gene(s) of the present disclosure is specific to plant seeds. In some embodiments, expression of the modified KTI gene(s) of the present disclosure is specific to soybeans of the soybean plant.

### Chloroplast Targeting

In some embodiments, the engineered plants can be engineered to contain modified chloroplast genes or to ensure expression in the chloroplast. In some embodiments, chloroplast transformation methods or compartmentalization of the modified KTI gene product encoding polynucleotides and/or polypeptides to the chloroplast. For instance, the introduction of genetic modifications in the plastid genome can reduce biosafety issues such as gene flow through pollen.

Methods of chloroplast transformation are known in the art and include Particle bombardment, PEG treatment, and microinjection. Additionally, methods involving the translocation of transformation cassettes from the nuclear genome to the plastid can be used as described in WO2010061186.

In some embodiments, one or more of the modified KTI gene product encoding polynucleotides can be targeted to the plant chloroplast. This can be achieved by incorporating in the expression construct a sequence encoding a chloroplast transit peptide (CTP) or plastid transit peptide, operably linked to the 5′ region of the sequence encoding the Cas protein. The CTP is removed in a processing step during translocation into the chloroplast. Chloroplast targeting of expressed proteins is well known to the skilled artisan (see for instance Protein Transport into Chloroplasts, 2010, Annual Review of Plant Biology, Vol. 61: 157-180). In such embodiments it is also can be desirable to target the guide RNA to the plant chloroplast. Methods and constructs which can be used for translocating guide RNA into the chloroplast by means of a chloroplast localization sequence are described, for instance, in US 20040142476, incorporated herein by reference. Such variations of constructs can be incorporated into the expression systems of the invention to efficiently translocate the modified KTI gene product encoding polynucleotides.

### Methods of Using the Engineered Soybean Plants and Soybeans

As previously discussed elsewhere herein, the engineered soybean plants and progeny thereof described herein can be grown, harvested and otherwise cultivated. Methods of growing, cultivating, and harvesting soybean plants is generally known and can be applied to engineered plants described herein.

The engineered soybean plants and/or parts thereof (e.g., soybeans) described herein can be harvested and utilized as a feed or food products. As such described in certain example embodiments herein are feed and/or food products comprising a soybean plant and/or soybean of the present description herein or a soybean product produced therefrom.

The feed and/or food products from the engineered soybean plants and/or soybeans described herein can be fed to animals or provided as a food to humans. As such, described in certain example embodiments herein are methods comprising feeding a feed and/or food product of the present description herein to a human or non-human animal.

### Methods and Kits for Identifying Low TI Plants

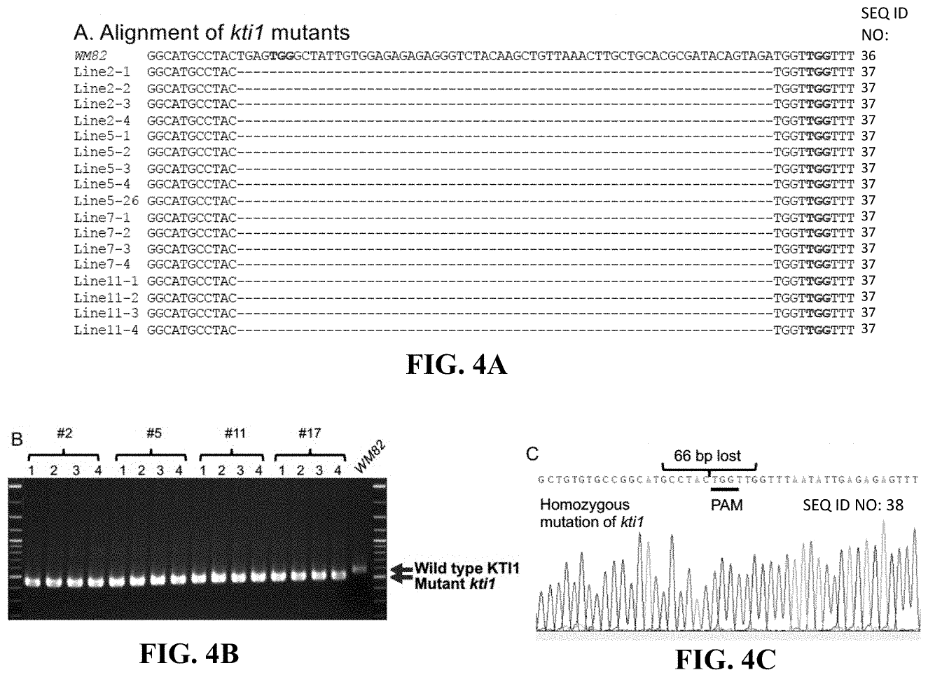

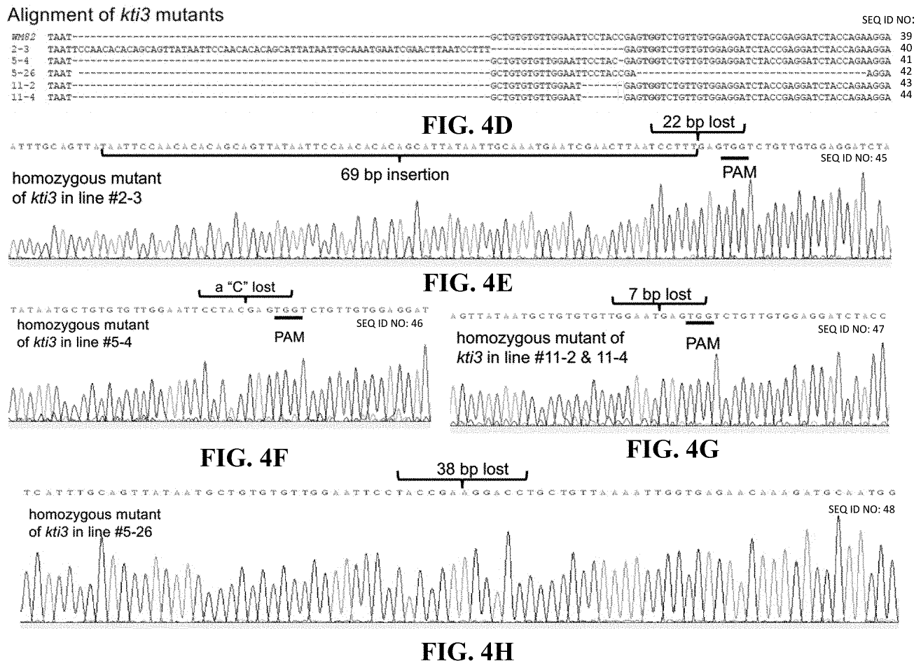

Described in certain example embodiments herein are methods for detecting biomarkers associated with mutant KTI1 and/or KTI3 genes that can convey reduced TI. In some embodiments, the biomarkers are modified KTI1 and/or KTI3 genes as demonstrated in the Working Examples herein (see e.g., FIG. 4A-4H). In some embodiments, the method includes specifically amplifying a gene fragment from a KTI1 and/or a KTI3 gene in a sample of DNA obtained from a soybean plant or part thereof to be tested. In some embodiments, primers used for amplification are configured to have one common primer to wild-type and modified KTI and/or modified KTI3 gene and one primer in each primer pair that specifically binds a mutant KTI gene or the wild type or control KTI gene such that mutants and wild-type/control amplicons can be distinguished. Exemplary primers are demonstrated in the Working Examples herein. This can allow for specific detection of biomarkers associated with reduced TI.

In some embodiments, a sequencing based assay can be used to detect a biomarker described herein. In some embodiments, the assay is a polymerase chain (PCR) based assay. Methods of harvesting, collecting, extracting and otherwise preparing an appropriate nucleic acid sample from a soybean plant or plant part for analysis by polymerase chain reaction will be appreciated by one of ordinary skill in the art in view of this disclosure. The amount of sample can be any suitable non-zero amount. In some embodiments the amount of sample can range from 0.1-1.0 pg, ng, microgram, g, kg, pL, nL, microliter, mL, L or more, such as 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99 to/or 1.00 pg, ng, microgram, g, kg, pL, nL, microliter, mL, L or more.

DNA extraction from soybean plants, plant parts, and other such samples can be carried out using a commercial method. For example, DNA extraction can be carried out using a Maxwell® 16 Cell LEV DNA Purification Kit. Other suitable commercial reagents, kits, and methods will be appreciated by one of ordinary skill in the art in view of this description herein. The nucleic acid sample can contain, for example, genomic DNA, cDNA, and/or RNA.

After obtaining the nucleic acid sample, a biomarker described herein (e.g., a modified KTI gene) can be specifically amplified and/or detected by performing a PCR based assay. The PCR based-assay can be one step or multiple step PCR assay. The PCR based assay can employ an appropriate primer set to specifically amplify an amplicon specific to a KTI biomarker. The PCR based assay can also employ appropriate primer sets to amplify one or more reaction and assay control genes, such as wild-type KTI genes, non-KTI genes (e.g., housekeeping genes), and/or other internal assay controls.

In some embodiments, the PCR-based assay is a real-time PCR assay. In some embodiments, the PCR-based assay is a reverse-transcriptase PCR assay. In some embodiments, the PCR-based assay is a quantitative PCR-based assay.

The PCR method employed in the assay herein can be any suitable PCR method. PCR methods include, but are not limited to, such as PCR, RT-PCR, nested-PCR, real-time PCR methods, multiplexed PRC, long-range PCR, single cell PCR, fast-cycling PCR, Methylation specific PCR, hot-start PCR, Hi-fidelity PCR, asymmetric PCR, overlap extension PCR, ligation mediated PCR, solid-phase PCR, touch down PCR, The PCR can be designed to amplify genomic DNA or be adapted to detect expression of a gene, such as RT-PCR. In some embodiments, the PCR method is qualitative. In some embodiments, the PCR method is quantitative. The PCR can be performed using any suitable machines, which are commercially available. Methods of interpreting results, qualitatively or quantitatively, are generally known in the art. See e.g., M. W. Pfaffl. “Relative quantification” pgs. 63-82 in Real-Time PCR ed. T. Dorak. Published by International University and Fronhoffs et al. 2002. Mol. Cell. Probes 16:99-110. In some embodiments, the PCR method includes generation of a standard curve (e.g., a standard cRNA or cDNA curve) such that molecules of amplicons produced from a sample can be quantified.

The PCR reaction can be carried out in any suitable reaction volume. In some embodiments, the reaction volume can range from or be 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20, 20.1, 20.2, 20.3, 20.4, 20.5, 20.6, 20.7, 20.8, 20.9, 21, 21.1, 21.2, 21.3, 21.4, 21.5, 21.6, 21.7, 21.8, 21.9, 22, 22.1, 22.2, 22.3, 22.4, 22.5, 22.6, 22.7, 22.8, 22.9, 23, 23.1, 23.2, 23.3, 23.4, 23.5, 23.6, 23.7, 23.8, 23.9, 24, 24.1, 24.2, 24.3, 24.4, 24.5, 24.6, 24.7, 24.8, 24.9, 25, 25.1, 25.2, 25.3, 25.4, 25.5, 25.6, 25.7, 25.8, 25.9, 26, 26.1, 26.2, 26.3, 26.4, 26.5, 26.6, 26.7, 26.8, 26.9, 27, 27.1, 27.2, 27.3, 27.4, 27.5, 27.6, 27.7, 27.8, 27.9, 28, 28.1, 28.2, 28.3, 28.4, 28.5, 28.6, 28.7, 28.8, 28.9, 29, 29.1, 29.2, 29.3, 29.4, 29.5, 29.6, 29.7, 29.8, 29.9, 30, 30.1, 30.2, 30.3, 30.4, 30.5, 30.6, 30.7, 30.8, 30.9, 31, 31.1, 31.2, 31.3, 31.4, 31.5, 31.6, 31.7, 31.8, 31.9, 32, 32.1, 32.2, 32.3, 32.4, 32.5, 32.6, 32.7, 32.8, 32.9, 33, 33.1, 33.2, 33.3, 33.4, 33.5, 33.6, 33.7, 33.8, 33.9, 34, 34.1, 34.2, 34.3, 34.4, 34.5, 34.6, 34.7, 34.8, 34.9, 35, 35.1, 35.2, 35.3, 35.4, 35.5, 35.6, 35.7, 35.8, 35.9, 36, 36.1, 36.2, 36.3, 36.4, 36.5, 36.6, 36.7, 36.8, 36.9, 37, 37.1, 37.2, 37.3, 37.4, 37.5, 37.6, 37.7, 37.8, 37.9, 38, 38.1, 38.2, 38.3, 38.4, 38.5, 38.6, 38.7, 38.8, 38.9, 39, 39.1, 39.2, 39.3, 39.4, 39.5, 39.6, 39.7, 39.8, 39.9, 40, 40.1, 40.2, 40.3, 40.4, 40.5, 40.6, 40.7, 40.8, 40.9, 41, 41.1, 41.2, 41.3, 41.4, 41.5, 41.6, 41.7, 41.8, 41.9, 42, 42.1, 42.2, 42.3, 42.4, 42.5, 42.6, 42.7, 42.8, 42.9, 43, 43.1, 43.2, 43.3, 43.4, 43.5, 43.6, 43.7, 43.8, 43.9, 44, 44.1, 44.2, 44.3, 44.4, 44.5, 44.6, 44.7, 44.8, 44.9, 45, 45.1, 45.2, 45.3, 45.4, 45.5, 45.6, 45.7, 45.8, 45.9, 46, 46.1, 46.2, 46.3, 46.4, 46.5, 46.6, 46.7, 46.8, 46.9, 47, 47.1, 47.2, 47.3, 47.4, 47.5, 47.6, 47.7, 47.8, 47.9, 48, 48.1, 48.2, 48.3, 48.4, 48.5, 48.6, 48.7, 48.8, 48.9, 49, 49.1, 49.2, 49.3, 49.4, 49.5, 49.6, 49.7, 49.8, 49.9, 50, 50.1, 50.2, 50.3, 50.4, 50.5, 50.6, 50.7, 50.8, 50.9, 51, 51.1, 51.2, 51.3, 51.4, 51.5, 51.6, 51.7, 51.8, 51.9, 52, 52.1, 52.2, 52.3, 52.4, 52.5, 52.6, 52.7, 52.8, 52.9, 53, 53.1, 53.2, 53.3, 53.4, 53.5, 53.6, 53.7, 53.8, 53.9, 54, 54.1, 54.2, 54.3, 54.4, 54.5, 54.6, 54.7, 54.8, 54.9, 55, 55.1, 55.2, 55.3, 55.4, 55.5, 55.6, 55.7, 55.8, 55.9, 56, 56.1, 56.2, 56.3, 56.4, 56.5, 56.6, 56.7, 56.8, 56.9, 57, 57.1, 57.2, 57.3, 57.4, 57.5, 57.6, 57.7, 57.8, 57.9, 58, 58.1, 58.2, 58.3, 58.4, 58.5, 58.6, 58.7, 58.8, 58.9, 59, 59.1, 59.2, 59.3, 59.4, 59.5, 59.6, 59.7, 59.8, 59.9, 60, 60.1, 60.2, 60.3, 60.4, 60.5, 60.6, 60.7, 60.8, 60.9, 61, 61.1, 61.2, 61.3, 61.4, 61.5, 61.6, 61.7, 61.8, 61.9, 62, 62.1, 62.2, 62.3, 62.4, 62.5, 62.6, 62.7, 62.8, 62.9, 63, 63.1, 63.2, 63.3, 63.4, 63.5, 63.6, 63.7, 63.8, 63.9, 64, 64.1, 64.2, 64.3, 64.4, 64.5, 64.6, 64.7, 64.8, 64.9, 65, 65.1, 65.2, 65.3, 65.4, 65.5, 65.6, 65.7, 65.8, 65.9, 66, 66.1, 66.2, 66.3, 66.4, 66.5, 66.6, 66.7, 66.8, 66.9, 67, 67.1, 67.2, 67.3, 67.4, 67.5, 67.6, 67.7, 67.8, 67.9, 68, 68.1, 68.2, 68.3, 68.4, 68.5, 68.6, 68.7, 68.8, 68.9, 69, 69.1, 69.2, 69.3, 69.4, 69.5, 69.6, 69.7, 69.8, 69.9, 70, 70.1, 70.2, 70.3, 70.4, 70.5, 70.6, 70.7, 70.8, 70.9, 71, 71.1, 71.2, 71.3, 71.4, 71.5, 71.6, 71.7, 71.8, 71.9, 72, 72.1, 72.2, 72.3, 72.4, 72.5, 72.6, 72.7, 72.8, 72.9, 73, 73.1, 73.2, 73.3, 73.4, 73.5, 73.6, 73.7, 73.8, 73.9, 74, 74.1, 74.2, 74.3, 74.4, 74.5, 74.6, 74.7, 74.8, 74.9, 75, 75.1, 75.2, 75.3, 75.4, 75.5, 75.6, 75.7, 75.8, 75.9, 76, 76.1, 76.2, 76.3, 76.4, 76.5, 76.6, 76.7, 76.8, 76.9, 77, 77.1, 77.2, 77.3, 77.4, 77.5, 77.6, 77.7, 77.8, 77.9, 78, 78.1, 78.2, 78.3, 78.4, 78.5, 78.6, 78.7, 78.8, 78.9, 79, 79.1, 79.2, 79.3, 79.4, 79.5, 79.6, 79.7, 79.8, 79.9, 80, 80.1, 80.2, 80.3, 80.4, 80.5, 80.6, 80.7, 80.8, 80.9, 81, 81.1, 81.2, 81.3, 81.4, 81.5, 81.6, 81.7, 81.8, 81.9, 82, 82.1, 82.2, 82.3, 82.4, 82.5, 82.6, 82.7, 82.8, 82.9, 83, 83.1, 83.2, 83.3, 83.4, 83.5, 83.6, 83.7, 83.8, 83.9, 84, 84.1, 84.2, 84.3, 84.4, 84.5, 84.6, 84.7, 84.8, 84.9, 85, 85.1, 85.2, 85.3, 85.4, 85.5, 85.6, 85.7, 85.8, 85.9, 86, 86.1, 86.2, 86.3, 86.4, 86.5, 86.6, 86.7, 86.8, 86.9, 87, 87.1, 87.2, 87.3, 87.4, 87.5, 87.6, 87.7, 87.8, 87.9, 88, 88.1, 88.2, 88.3, 88.4, 88.5, 88.6, 88.7, 88.8, 88.9, 89, 89.1, 89.2, 89.3, 89.4, 89.5, 89.6, 89.7, 89.8, 89.9, 90, 90.1, 90.2, 90.3, 90.4, 90.5, 90.6, 90.7, 90.8, 90.9, 91, 91.1, 91.2, 91.3, 91.4, 91.5, 91.6, 91.7, 91.8, 91.9, 92, 92.1, 92.2, 92.3, 92.4, 92.5, 92.6, 92.7, 92.8, 92.9, 93, 93.1, 93.2, 93.3, 93.4, 93.5, 93.6, 93.7, 93.8, 93.9, 94, 94.1, 94.2, 94.3, 94.4, 94.5, 94.6, 94.7, 94.8, 94.9, 95, 95.1, 95.2, 95.3, 95.4, 95.5, 95.6, 95.7, 95.8, 95.9, 96, 96.1, 96.2, 96.3, 96.4, 96.5, 96.6, 96.7, 96.8, 96.9, 97, 97.1, 97.2, 97.3, 97.4, 97.5, 97.6, 97.7, 97.8, 97.9, 98, 98.1, 98.2, 98.3, 98.4, 98.5, 98.6, 98.7, 98.8, 98.9, 99, 99.1, 99.2, 99.3, 99.4, 99.5, 99.6, 99.7, 99.8, 99.9, to/or 100 pL, nL, microliter, mL or more. The PCR reaction can be carried out in any suitable reaction vessel, microfluidic vessel, or on any suitable substrate that is capable of maintaining segregated, addressable droplets.

In some embodiments, such as those employing real-time PCR methods of gene fragment amplification, an oligonucleotide probe specific to the target polynucleotide (e.g., gene or transcript) and primer set can be used. An example of such a probe is a TaqMan® probe. Different genes can be detected in the same reaction using TaqMan® probes by using a different fluorophore on each probe so that the gene amplification from each gene can be differentiated based on the fluorescence produced. Quencher molecules can be fluorescent (e.g., TAMRA™) or nonfluorescent molecules e.g., DABCYL and Black Hole Quencher®). Example fluorophores include, but are not limited to 6-FAM™, JOE™, TET™, Cal Fluor® Gold 540, HEX™, Cal Fluor® Orange 560, TAMRA™, VIC™, Cyanine 3, Quasar® 570, Cal Fluor® Orange 590, ROX™, Texas Red®, Cyanine 5, Quasar® 670, and Cyanine 5.5.

In some embodiments, including but not limited to those employing real-time PCR methods of gene fragment amplification, a fluorescent dye that binds to double stranded DNA can be used to monitor gene fragment amplification during each PCR reaction cycle. An example of such a dye is SYBR® Green and its variants. In some embodiments, the detectable dye molecule that binds double stranded DNA is a cyanine dye. In some embodiments, the detectable dye molecule that binds double stranded DNA is N′,N′-dimethyl-N-[4-[(E)-(3-methyl-1,3-benzothiazol-2-ylidene)methyl]-1-phenylquinolin-1-ium-2-yl]-N-propylpropane-1,3-diamine and variants thereof.

In assays where multiple genes are amplified in a single reaction (also referred to in the art as a “multiplexed reaction” or simply “multiplexing”) where a fluorescent dye that binds to double stranded DNA is used to monitor amplification of gene fragments, the amount (relative or quantitative) of gene fragments amplified from the different genes present in the reaction can be distinguished from each other by virtue of different melting curves. Primer sets can be designed for each gene in the multiplex reaction such that the gene fragment generated will disassociate at a different temperature and thus produce a different melting curve. This can allow for multiplexing in reactions where amplicon specific probes are not employed.

It will be appreciated that the step of amplifying can contain at least two or more cycles where the number of amplicons is approximately doubled each cycle. The exact steps within each cycle will vary depending on the PCR technique used, but all include separating or partially separating a double stranded DNA molecule to create single stranded DNA molecules contacting the resulting template single stranded DNA molecule(s) with one or more primers and a DNA polymerase, and allowing the polymerase to incorporate free nucleotides into a new strand that is an extension of the primer that is bound to the template DNA molecule, thus forming a new double stranded DNA molecule. This is repeated for a desired number of cycles to generate a pool of amplicons. In some embodiments, the number of cycles can range from about 2 to about 70, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70. In some embodiments, the number of cycles performed is 28, 29, 30, 31, 32, 33, 34, 35, 36, 37, 38, 39, 40, 41, 42, or 43. In some embodiments, the PCR method can include an initial denaturation step or other steps before the amplification cycles begin. In some embodiments, the PCR method can include other steps and processes after the final amplification cycle occurs, such as a final extension step or melting curve analysis. In some embodiments, a melting curve analysis can be performed as amplification is occurring at each cycle.

Described in certain example embodiments herein are kits for identifying soybean plants having reduced trypsin inhibition as compared to a suitable control, the kit comprising (a) a set of primers configured to amplify a region of a KTI1 mutant gene, (b) a set of primers configured to amplify a region of a KTI3 mutant gene, or (c) both (a)-(b), and a set of primers configured to amplify a region of a wild-type or control soybean KTI1 gene, a set of primers configured to amplify a region of a wild-type or control soybean KTI3 gene, or both, wherein the amplicon generated from the set of primers in (a) is different in size and/or sequence as compared to the amplicon generated from the set of primers configured to amplify a region of a wild-type or control soybean KTI1 gene, and wherein the amplicon generated from the set of primers in any one of (b) are different in size and/or sequence as compared to the amplicon generated from the set of primers configured to amplify a region of a wild-type or control soybean KTI3 gene. An exemplary assay is shown in FIG. 7A-7B.

In some embodiments, a PCR method is used to amplify the gene fragments/regions.

In certain example embodiments, the method further comprises one or more reagents suitable for isolating, preparing, amplifying, and/or sequencing nucleic acids. The kits can contain one oligonucleotide probes, dyes, etc. and other buffers, reagents etc. used in performing a PCR-based assay, such as any of those described herein. The kit can also contain any containers (e.g., vessels, microwell strips or plates, etc.) that can be used in performance of the PCR-based assay.

In certain example embodiments, the wild-type soybean KTI1 gene has a sequence according to SEQ ID NO: 1, the wild-type soybean KTI3 gene has a sequence according to SEQ ID NO: 3.

In certain example embodiments, the set of primers of (a) and the set of primers configured to amplify a region of a wild-type or control soybean KTI1 gene share a common forward primer or a common reverse primer, wherein the set of primers of (b) and the set of primers configured to amplify a region of a wild-type or control soybean KTI3 gene share a common forward primer or a common reverse primer, or both.

In certain example embodiments, the kit comprises one or more primers each having a sequences according to one of SEQ ID NO: 26-30.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

### Introduction

Soybean meal provides an excellent source of protein in animal feed since it is rich in amino acids with a high nutritional profile (Cromwell, Stahly et al. 1991). For instance, soy makes up 26% and 50% of swine and poultry feed, respectively (Gillman, Kim et al. 2015). However, reduced feed efficiency has been observed due to anti-nutritional and biologically active factors in raw soybean seeds (Liener 1996). Among these factors, trypsin inhibitor (TI) accounts for a substantial amount of this effect that cannot be ignored (Hymowitz 1986). TI restrains the activity of trypsin in monogastric animals. Because this enzyme is essential for optimal protein digestion, its restriction can lead to animal growth inhibition of 30-50% due to pancreatic hypertrophy/hyperplasia when raw soybeans are used in feed (Hymowitz 1986, Cook, Jensen et al. 1988, Liener 1994). In soybean meal processing facilities, TI in soybean meal is deactivated via a heating process at 90.5° C.-100° C. with the presence of 1% NaOH (Chen, Xu et al. 2014). This process not only reduces the nutritional value of soybean meal due to thermal destruction of amino acids, but also increases the energy cost of meal production by 25% (Chang, Tanksley et al. 1987).

With the recent increase in feed price and shipping cost, livestock farmers and grain operations are reconsidering soybean varieties with low-TI or TI-free traits as a way to reduce farm expenses by using raw soybeans as feed. Raising low-TI or TI-free soybeans on farms creates a niche market for integrated crop and livestock farmers, increasing their farm's profitability. The use of soybean lines with genetically reduced levels of TI has proven as an effective strategy for improving animal growth. For instance, chicks fed soy-based diets with raw, unprocessed, low-KTI soymeal had higher feed efficiency ratios than chicks fed diets containing raw, unprocessed, conventional soybean meal (Batal and Parsons 2004). Thus, soybean cultivars with low TI content in the seeds is a long-term breeding goal for higher protein digestibility, better economic benefits, reduced environmental pollution caused by phosphorus, and the pursuit of sustainability for humanity and nature.

Plants have evolved a group of TI genes encoding proteins that can suppress the enzyme activities of proteases found in in plants, herbivores, animals and human beings (Jofuku and Goldberg 1989, Schuler, Poppy et al. 1999). The TIs in soybean can be classified into two families: the 21 kDa Kunitz trypsin inhibitor protein family (KTI) and the 7-8 kDa Bowman-Birk inhibitor protein family (BBTI) (Kunitz 1945, Wei 1983, Gillman, Kim et al. 2015). KTI proteins are thought to be largely specific for trypsin inhibition, while the major isoform of BBTI contains domains that interact with and inhibit both trypsin and chymotrypsin (Hwang, Foard et al. 1977, Gillman, Kim et al. 2015). Currently, only the KTI genes are targeted for selection of low-TI soybeans because KTI serves as the major contributor to trypsin inhibitor activity in soybeans. By far, the most significant success in reducing TI activity in soybean was the identification of a soybean accession (PI 157740) with dramatically reduced (˜40%) TI activity (Gillman, Kim et al. 2015). A frameshift mutation in KTI3 (Gm08g341500) gene was identified in PI 157740, which is responsible for the low TI phenotype (Jofuku, Schipper et al. 1989). PI 157740 has been used in feeding trials, and it was found that raw extruded protein meal with lower KTI3 protein is superior for animal weight gain when compared to raw soybean meal harboring functional KTI3 (Cook, Jensen et al. 1988, Perez-Maldonado, Mannion et al. 2003). However, weight gain for young animals fed with non-heat-treated soybean materials including nonfunctional KTI3 soybean materials is still inferior to those fed with heat-treated soybeans (Cook, Jensen et al. 1988, Perez-Maldonado, Mannion et al. 2003). Another soybean germplasm accession (PI 68679) was identified to carry a nonfunctional mutation on KTI1 (Gm01g095000) gene (Gillman, Kim et al. 2015). KTI1 and KTI3 genes were determined to be synergistically controlling the TI content in soybean seeds (Gillman, Kim et al. 2015). Therefore, it is desirable to breed new soybean cultivars carrying both kti1 and kti3 mutant alleles. However, it is time-consuming to breed low TI soybean cultivars by selecting progenies derived from crosses between PI 157740, PI 68679, and elite varieties. Besides, the linkage drags associated with KTI1 and KTI3 may introduce undesirable agronomic traits, which could be difficult to remove by backcrossing. Although Kompetitive Allele Specific PCR (KASP) markers associated with KTI3 and its mutant allele with 86% efficiency are available (Rosso, Shang et al. 2021), attempts at developing molecular markers associated with KTI1 have not been successful (Gillman, Kim et al. 2015). Therefore, it is highly desirable to develop new KTI1 mutant alleles that can be tagged with convenient molecular markers.

CRISPR/Cas9 mediated genome editing employs a Cas9 endonuclease and an 18-22 bp small guide RNA (sgRNA) that have a region that is complementary to a target gene sequence. The sgRNA binds to Cas9 and recruits the complex to target a gene. The Cas9 endonuclease generates DNA breaks, leading to mis-repaired target genes that contain deletions or insertions that disrupt gene function. In addition, several sgRNAs can be co-expressed in a single cell with Cas9, which allows the multiplex mutations of different genes simultaneously (Liang, Zhang et al. 2016). Because genome edited plants without transgenes are not considered as genetically modified organisms (GMO) (Kim and Kim 2016), mutant plants can be either directly released for field test or served as valuable resources for further breeding selection. Thus far, the CRISPR/Cas9 mediated genome editing technology has been widely used for targeted gene mutagenesis in diverse crop plant species, including soybean (Haun, Coffman et al. 2014, Jacobs, LaFayette et al. 2015, Cai, Chen et al. 2018), rice (Xu, Li et al. 2015), wheat (Upadhyay, Kumar et al. 2013), maize (Chen, Xu et al. 2018), tomato (Vu, Sivankalyani et al. 2020), cotton (Gao, Long et al. 2017), citrus (Peng, Chen et al. 2017), apple (Osakabe, Liang et al. 2018), grape (Osakabe, Liang et al. 2018), potato (Nakayasu, Akiyama et al. 2018), and banana (Shao, Wu et al. 2020) to improve their agronomic performances. For example, Jacobs et al. (2015) reported the first targeted mutagenesis in soybean using the CRISPR/Cas9 technology (Jacobs, LaFayette et al. 2015). Haun et al. (2014) generated a high oleic acid content soybean variety without transgenic components and improved the quality of soybean (Haun, Coffman et al. 2014). A soybean mutant with a late flowering phenotype was created using CRISPR/Cas9 technology to knock out the GmF7-2a gene (Cai, Chen et al. 2018). Thus far, there is no research reporting an attempt to apply CRISPR/Cas9 to target anti-nutritional factors in soybean.

In this study, Applicant aimed to (1) simultaneously knockout KTI1 and KTI3 genes in soybean cultivar Williams 82 via CRISPR/Cas9-mediated genome editing and (2) develop molecular markers associated with kti1 and kti3 mutant alleles that can be used for marker-assisted selection (MAS). Applicant successfully recovered transgenic soybean plants that are carrying both kti1 and kti3 mutations. KTI content and trypsin inhibition activities (TIA) are dramatically decreased in the kti1 and kti3 mutant lines. In addition, Applicant also developed molecular markers for co-selection of the new kti1 and kti3 mutant alleles. These kti1 and kti3 mutant lines and the newly developed selection markers have great potential for breeding the low TI trait into elite soybean varieties in the future.

### Methods

### Plant Materials and Growth Conditions

Soybean plants were grown in 2.5-gallon pots using Miracle-Gro all-purpose potting soil mix in Keck Greenhouse at Virginia Tech (14 h/10 h light/dark cycle at 25° C./20° C.) for the experiments described herein. The plants were watered by an automatic irrigation system. Soybean transformation was performed at the plant transformation facility at Iowa State University as previously described (Paz, Martinez et al. 2006, Luth, Warnberg et al. 2015, Ge, Yu et al. 2016). The plant growth indicators and maturity period days of WM82 and progeny plants of the T1 generation derived from lines #2 and #5 were measured in the green house. The 4-week-old T1 soybean plants were used for genotyping. The seeds of T1 plants were used for seed weight analysis.

### Constructing a Soybean KTI Gene Map

The gene map showing locations of KTI1 genes on soybean chromosomes was made using MapInspect. Locations of all KTI1 genes were obtained from the Phytozome database and plotted on their respective chromosomes.

### Bacterial Growth

E. coli strains DH5α and C41 (DE3) (Lucigen, Middleton, WI) were grown on Luria agar medium at 37° C. Agrobacterium tumefaciens (A. tumefaciens) EHA105 was grown on Luria agar medium at 28° C. (Zhao, Dahlbeck et al. 2011). E. coli antibiotic selections used in this study were as follows: 50 μg/ml kanamycin, 100 μg/ml carbenicillin, 100 μg/ml spectinomycin. A. tumefaciens antibiotic selection were 100 μg/ml rifampicin, and/or 100 μg/ml spectinomycin.

### Cloning

The open reading frames (ORFs) of KTI1 and KTI3, were amplified from the genomic DNA of WM82. The KTI1_{Δ66 bp}, truncated ORF of KTI1, was amplified from the genomic DNA of mutant soybean plant #2-1. All PCR primers with annotations are listed in Table 2. The genes/fragments were then cloned into a pDonr207 plasmid (Thermo Fisher Scientific) for future use.

T1 plant genomic DNA (gDNA) was used as the templates to amplify KTI1 and/or its mutant allele, and KTI3 and/or its mutant allele by PCR. The purified PCR fragments were used for genotyping by Sanger sequencing at Virginia Tech Genomic Sequencing Center and cloned to the PCR8/GW/TOPO vector by TA cloning (Invitrogen) for molecular marker tests.

In order to apply the CRISPR/Cas9 system to gene editing in soybean, Applicant modified Applicant's current CRISPR/Cas9 construct (Liu, Miao et al., 2016). The cassette consists of a MAS promoter, the bialaphos resistant gene, and a MAS terminator that was amplified using plasmid DNA of pEarleyGate101 as the template. All PCR primers with annotations are listed in Table 2. The cassette was assembled to the backbone of CRISPR/Cas9 construct using Gibson Assembly® Cloning Kit (New England Biolabs Inc). The gRNAs targeting KTI1 and KTI3 were synthesized in one cassette at GenScript Biotech Corp. The backbone of the new CRISPR/Cas9 construct and the fragment of gRNAs were assembled together using Gibson Assembly® Cloning Kit.

TABLE 2
Oligo primers used in this Example

| | | SEQ ID | |
|---|---|---|---|
| Primer | Sequence | NO: | Annotation |
| KTI1 For | GGGGACAAGTTTGTACAAAAAAGCAGGCTtgATGAAG | 10 | Forward primer |
| | AGTACTATCTTCTTTG | | for amplifying |
| | | | KTI1-1 ORF for |
| | | | cloning |
| KTI1 Rev | GGGGACCACTTTGTAcaAGAAAGCTGGGTaTGCAGTT | 11 | Reverse primer |
| | GATGATCTAAATTTC | | for amplifying |
| | | | KTI1-1 ORF for |
| | | | cloning |
| KTI3 For | GGGGACAAGTTTGTACAAAAAAGCAGGCTtgATGAAG | 12 | Forward primer |
| | AGCACCATCTTCTT | | for amplifying |
| | | | KTI1-1 ORF for |
| | | | cloning |
| KTI3 Rev | GGGGACCACTTTGTAcaAGAAAGCTGGGTaCTCACTG | 13 | Reverse primer |
| | CGAGAAAGGCCATG | | for amplifying |
| | | | KTI1-1 ORF for |
| | | | cloning |
| MAS pro For | ATCCGTAGCATACTAGCATCTATCAGCTAGCgtttaaacC | 14 | Forward |
| | GGCTACCGATCGACTGACTAGCATGATGATaaacTTTT | | primer for |
| | CAAATCAGTGCGCAAGACG | | amplifying the |
| | | | cassette for |
| | | | soybean |
| | | | transformation |
| | | | selection |
| | | | Consists of |
| | | | MAS |
| | | | promoter, Bar |
| | | | gene, and MAS |
| | | | terminator. |
| MAS ter Rev | cgatctagtaacatagatgacaccgcgcgcgGATAATTTATTTGAA | 15 | Reverse |
| | AATTCATAAGA | | primer for |
| | | | amplifying the |
| | | | cassette for |
| | | | soybean |
| | | | transformation |
| | | | selection. |
| | | | Consists of |
| | | | MAS |
| | | | promoter, Bar |
| | | | gene, and MAS |
| | | | terminator |
| KTI1 | TCCTCTTCAAAACGGTGGCA | 16 | Forward primer |
| (Gm01G095000) | | | for amplifying |
| real-time PCR For | | | Gm01G095000 |
| | | | in real- |
| | | | time PCR |
| KTI1 | GTATCGCGTGCAGCAAGTTT | 17 | Reverse primer |
| (Gm01G095000) | | | for amplifying |
| real-time PCR Rev | | | Gm01G095000 |
| | | | in real- |
| | | | time PCR |
| Gm08G342300 | GGAGTCGCTTTCATCACCCA | 18 | Forward primer |
| real- | | | for amplifying |
| time PCR For | | | Gm08G342300 |
| | | | in real- |
| | | | time PCR |
| Gm08G342300 | AAGCGCCTGATTCCCTTACC | 19 | Reverse primer |
| real- | | | for amplifying |
| time PCR Rev | | | Gm08G342300 |
| | | | in real- |
| | | | time PCR |
| Gm08G341000 | ACCTGGTGTTTGGATGTCGG | 20 | Forward primer |
| real- | | | for amplifying |
| time PCR For | | | Gm08G341000 |
| | | | in real- |
| | | | time PCR |
| Gm08G341000 | ACGGTTCACCAGTAACAGCA | 21 | Reverse primer |
| real- | | | for amplifying |
| time PCR Rev | | | Gm08G341000 |
| | | | in real- |
| | | | time PCR |
| KTI3 | ATGAAGGTAACCCTCTTGAAAATG | 22 | Forward primer |
| (Gm08G341500) | | | for amplifying |
| real-time PCR For | | | Gm08G341500 |
| | | | in real- |
| | | | time PCR |
| KTI3 | ACAACAGACCACTCGGTAGG | 23 | Reverse primer |
| (Gm08G341500) | | | for amplifying |
| real-time PCR Rev | | | Gm08G341500 |
| | | | in real- |
| | | | time PCR |
| ELF1B | GTTGAAAAGCCAGGGGACA | 24 | Forward |
| (housekeeping | | | primer for |
| gene) real-time | | | amplifying |
| PCR For | | | ELF1B in |
| | | | real-time |
| | | | PCR |
| ELF1B | TCTTACCCCTTGAGCGTGG | 25 | Reverse |
| (housekeeping | | | primer for |
| gene) real-time | | | amplifying |
| PCR Rev | | | ELF1B in |
| | | | real-time PCR |
| KTI1 selection | GGCTATTGTGGAGAGAGAGGG | 26 | ZW2, forward |
| forward | | | primer for wild |
| mutant | | | type KTI1 allele |
| KTI1/kti1 | CCATCATCGTCGATCTGAATC | 27 | ZW1, common |
| selection | | | reverse primer |
| common rev | | | |
| kti1 selection | GTGCCGGCATGCCTTGGT | 28 | ZW3, forward |
| forward | | | primer for |
| wild type | | | mutant kti1 |
| | | | allele |
| KTI3 selection | CTACCGAGTGGTCTGTTGTG | 29 | ZW5, forward |
| forward | | | primer for wild |
| mutant | | | type KTI3 allele |
| KTI3/kti3 | TGAAACTGAACCACTAACGGT | 30 | ZW4, common |
| selection | | | reverse primer |

### Expression Analysis of KTI Genes in WM82

RNA sequencing data, in FPKM (fragments per kilobase of transcript per million fragments mapped), of 38 KTI genes in 31 different tissue types from Williams 82 were acquired through the Gene Networks in Seed Development database (http://seedgenenetwork.net/sequence). Construction of the heatmap to visualize expression data was done using the heatmap.2 function from the ggplot2 package in R. A green/blue color gradient was chosen to show expression with blue representing little to no expression and green representing high expression. The code for the heatmap is as follows: heatmap.2(x=KTI Expression, main=“KTI Expression In Different Soybean Tissue”, notecol=“black”, density.info=“none”, trace=“none”, margins=c(12,9), col=my_palette, breaks=col_breaks, dendrogram=“row”, Colv=“NA”, ylab=“Genes”, xlab=“Tissue Type”, cexCol=0.9, cexRow=0.8)

### RNA Isolation and Real-Time PCR

All RNA was extracted from V98-9005 and V03-5903 seeds using TRIzol reagent (Thermo Fisher Scientific) according to the manufacturer's instructions. Any DNA residue was eliminated by treating with UltraPure DNase I (Thermo Fisher Scientific). The integrity and quantity of total RNA were determined by electrophoresis in 1% agarose gel and a NanoDrop ND-1000 spectrophotometer (NanoDrop Technologies, Wilmington, DE). cDNA synthesis was performed using the SuperScript III First-Strand RT-PCR Kit (Thermo Fisher Scientific) with an oligo-dT primer based on the manufacturer's instructions. Real-time PCR was conducted with cDNA as the template using the Quantitect SYBR Green PCR kit (Qiagen) according to the manufacturer's protocol. Oligo primers are listed in Table 2. The soybean ELF1B gene was used as reference gene, and data is presented as ACT (Jian, Liu et al. 2008).

Expression and Purification of KTI1, KTI1_{Δ22m}, and KTI3 proteins

The KTI1, KTI1_{Δ66 bp}, and KTI3 genes in pDonr207 were subcloned into a Gateway compatible pET28a destination vector via a LR® Gateway cloning kit (Thermo Fisher Scientific) (Earley, Haag et al. 2006, Liu, Miao et al. 2016). The plasmids were transformed into E. coli C41 cells (Lucigen). KTI1, KTI1_{Δ22aa}, and KTI3 proteins were expressed and purified following a procedure as previously described [43]. Protein purity was evaluated by SDS-PAGE. The protein concentration was determined by a protein assay kit (Bio-Rad) using bovine serum albumin as standard (Han, Zhou et al. 2015).

### Standard Bioassay to Measure Trypsin Inhibitor Activity

A TI activity bioassay was performed following American Association of Cereal Chemists Official Method 22-40 (AACC, 1999) with some modifications previously reported by Rosso et al. (2018). Briefly, 30 mg of finely ground soybean seed powder was mixed with 3 mL of 9 mM HCl (pH 2.0). The mixture was shaken for 1 h at room temperature. 2 mL of the extracts was centrifuged at 10,350 rpm for 20 min at room temperature, and the supernatant was diluted by 10 times with 9 mM HCl for measuring TI activity. A TI activity assay was performed in a 96-well plate format following the same steps described in Rosso et al. (2018). Each sample row was repeated three times. Portions of diluted HCl extracts (0, 20, 30, 40, and 60 μL) or 50 recombinant proteins of KTI1, KTI1A22aa, and KTI3 were pipetted into the microplate wells, and the volume was adjusted to 60 μL with 9 mM HCl. 60 μL of extractant was used as a sample blank and 60 μL of water were used as a substrate blank. 60 μL of trypsin (from bovine pancreas, Sigma-Aldrich T8003) solution was added to each sample well, and the microplates were placed in an oven at 37° C. for 15 min. After the incubation, 150 μL of BAPNA substrate pre-warmed at 37° C. was added to all wells, and the plates were incubated for exactly 10 min at 37° C. The reaction was stopped by adding 30 μL of acetic acid solution to all wells. The absorbance of each well was read on a plate reader (FLOUstar Omega, BMG Labtech) at 410 nm for 30 s after shaking at 700 rpm.

### HPLC Method to Quantify Kunitz Trypsin Inhibitor

The HPLC method to quantify ICH was performed following the method developed by Rosso et al. (2018). Briefly, 10 mg of finely ground soybean seed powder was mixed with 1.5 mL of 0.1 M sodium acetate buffer (pH 4.5). Samples were vortexed and shaken for 1 h at room temperature. The sample was centrifuged at 12,000 rpm for 15 min. 1 mL of the supernatant was filtered through a syringe with an IC Millex-LG 13-mm mounted 0.2-mm low protein binding hydrophilic millipore (polytetrafluoroethylene [PTFE]) membrane filter (Millipore Ireland). The KTI in solution was separated on an Agilent 1260 Infinity series (Agilent Technologies) equipped with a guard column (4.6×5 mm) packed with POROS R2 10-mm Self Pack Media and a Poros R2/H perfusion analytical column (2.1×100 mm, 10 um). The mobile Phase A consisted of 0.01% (v/v) trifluoroacetic acid in Milli-Q water, and the mobile Phase B was 0.085% (v/v) trifluoroacetic acid in acetonitrile. The injection volume was 10 mL and the detection wavelength was 220 nm.

Development of Molecular Selection Markers with a Gel Electrophoresis Free Method for High Throughput Screening

The transgene free and double homozygous mutant line, #5-26, was selected for the development of molecular selection markers. Based on the genotyping data of #5-26, two pairs of markers were designed: ZW1 with ZW2 or ZW3. ZW1 is the common reverse primer for both KIP and kti1, while ZW2 and ZW3 are two reverse primers matched with unique sequences in KTI1 and kti1, respectively. Similarly, two pairs of molecular markers, ZW4 with ZW5 or ZW6, were designed. ZW4 is the common reverse primer for both KTI3 and kti3, while ZW5 and ZW6 are two reverse primers matched with unique sequences in KTI3 and kti3, respectively. The gDNA of WM82, #5-26 (homozygous mutants of both kti1 and kti3), #5-9 (homozygous mutant of kti1 while heterozygous mutant of kti3) and #2-30 (homozygous mutant of kti3 while heterozygous mutant of kti1) were used as templates to test the efficiency and reliability of these markers in PCR.

PCR amplifications were performed in a total volume of 20 μl containing 50 ng of gDNA, 0.5 μM each of forward and reverse primers (Table 2), 10 μl 2×BioMix Red (Bioline) and ddH₂O. The PCR program was set to be 95° C. for 5 min for pre-denature, followed by 35 cycles of denaturation at 95° C. for 30 s, annealing at 55° C. for 30 s, extension at 72° C. for 30 s, followed by final extension at 72° C. for 5 min.

In order to screen the large-scale progenies derived from crosses between soybean elite cultivars carrying wild type KTI1/3 and the newly developed mutant plant carrying kti1/3, a simple gel electrophoresis-free method was designed. 1× sybrgreen dye (Thermo Fisher) was added to complete PCR reactions, and the solution was incubated for 10 mins at 75° C. before placing in the gel doc (Biorad) for imaging the fluorescent signals. Only the positive PCR products with the dye will display fluorescent signals while the failed PCR will not show signals.

### Statistical Data Analysis

Analytical experiments were performed with at least three technical replicates. Statistical significance was based on one-way ANOVA test for multiple comparisons. Data was analyzed using JMP Pro14. Values of P<0.05 were considered significant.

### Results

KTI Gene Family Consists of Multiple Members with Distinct Expression Patterns.

Soybean KTI genes belong to a gene family with thirty-eight members (Soybase version Wm82.a4.v1) (FIG. 1). As displayed in the genetic map, 38 KTI genes are located on 9 chromosomes (Chr) in the soybean genome. Specifically, Chr1 carries 2 KTI genes; Chr3 carries 1 KTI gene; Chr6 carries 1 KTI gene; Chr8 carries 13 KTI genes; Chr8 carries 10 KTI genes; Chr12 carries 1 KTI gene; Chr16 carries 8 KTI genes; Chr18 carries 1 KTI gene; and Chr19 carries 1 KTI gene (FIG. 1).

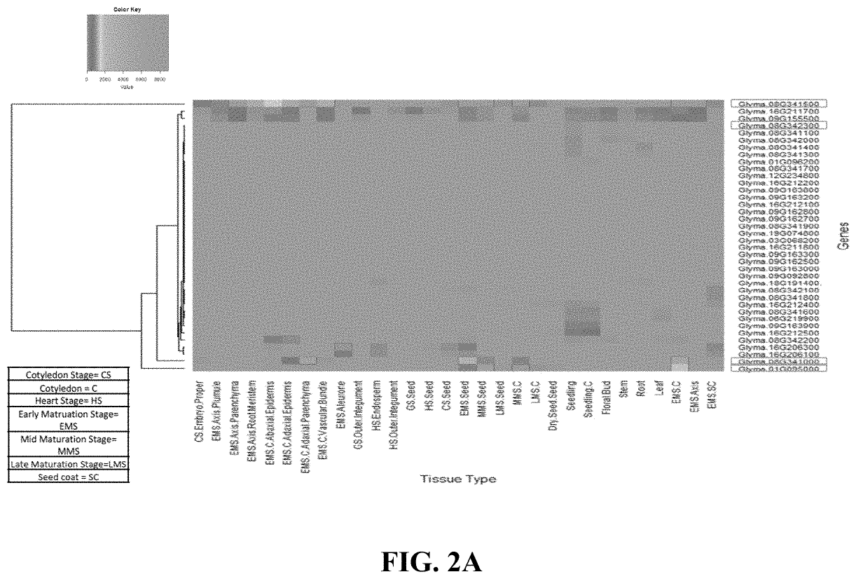

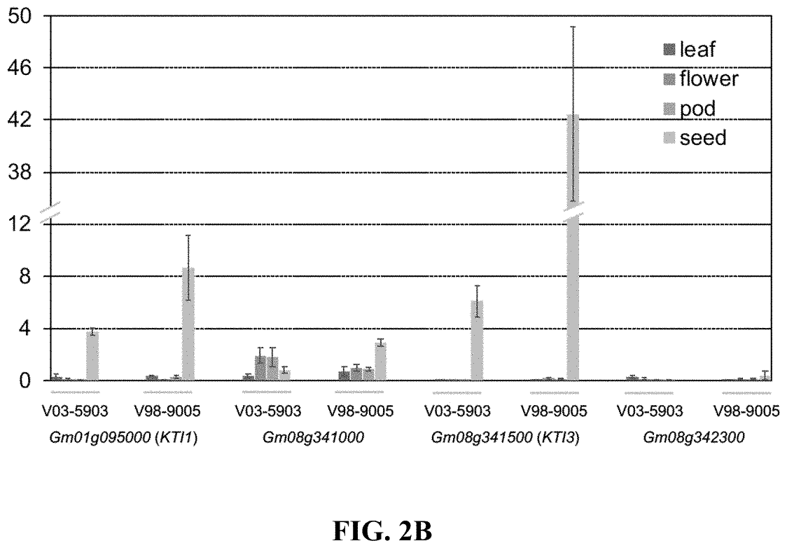

To identify the KTI genes expressed in the seed, Applicant analyzed the expression patterns of all KTI genes in cv. WM82 based on the expression data deposited in USDA ARS's Soybase (FIG. 2A). According to the expression patterns of KTI genes in various soybean tissues as displayed in FIG. 2A, three KTI genes Gm01g095000 (KTI1), Gm08g341000, Gm08g342300, and Gm08g341500 (KTI3) were identified as seed-specific KTI genes. Soybean breeding lines V98-9005 (normal TI) and V03-5903 (low TI), presenting significantly different amounts of KTI concentration in seeds, were used to validate the tissue specific expressions of the four KTI genes by real-time PCR. Gm01g095000 (KTI1) and Gm08g341500 (KTI3) were predominately expressed in seeds compared to other tissues (FIG. 2B). Both Gm08g341000 and Gm08g342300 had a relatively lower expression level in seeds than KTI1 and KTI3 but had higher expression in other tissue types (FIG. 2B). Interestingly, both KTI1 and KTI3 had a relatively low expression level in the seeds of V03-5903 (low TI line), but higher expression in V98-9005 (normal TI line). Thus, Applicant conclude that KTI1 and KTI3 are two major genes that may directly contribute to the TI contents in soybean seeds.

### Development of CRISPR/Cas9-Based Binary Vector for Genome-Editing in Soybean

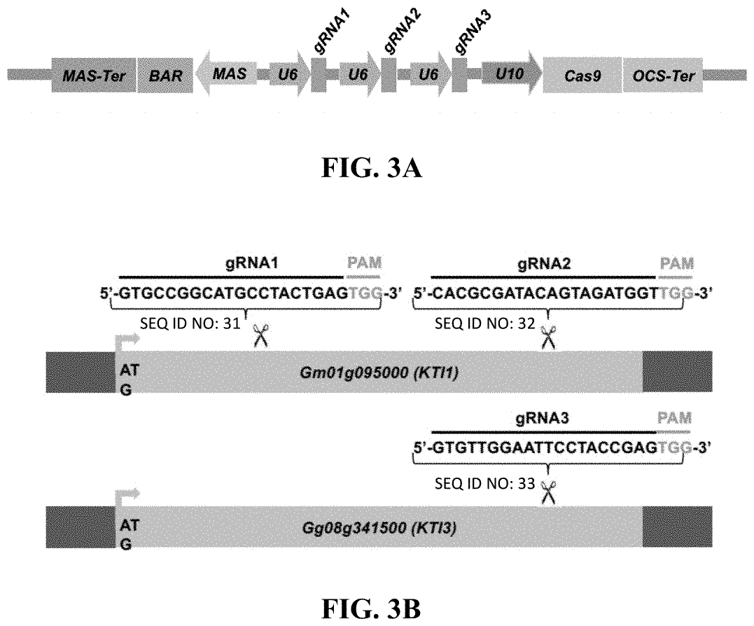

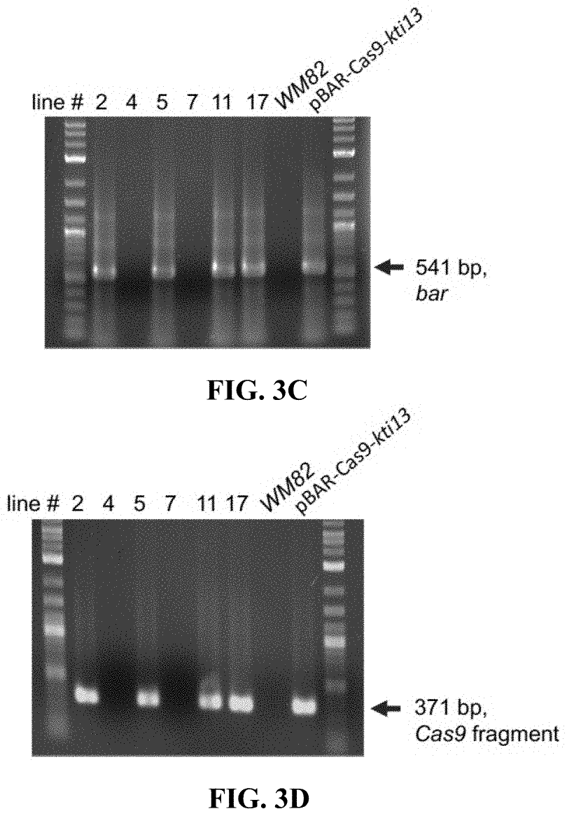

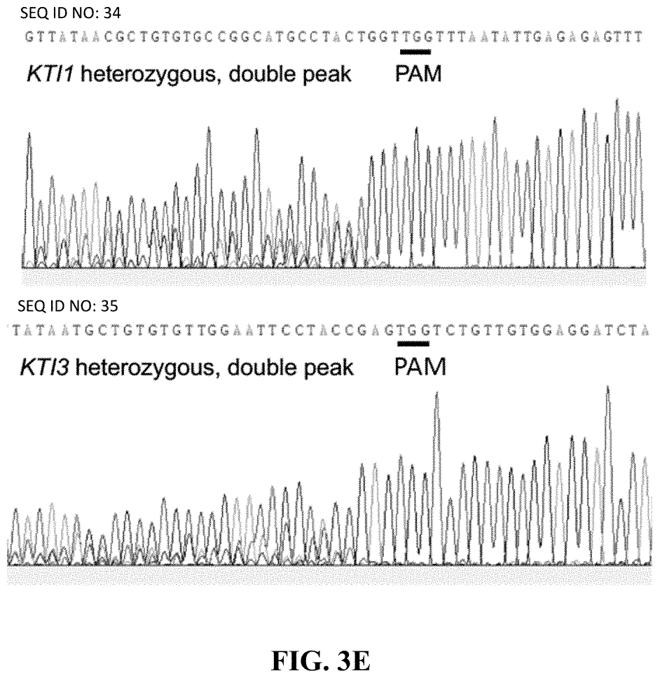

To knock out the KTI1 and KTI3 genes from cv. WM82 genome and create a new soybean cultivar with low TI content in soybean seeds, Applicant developed a CRISPR/Cas9 construct, pBAR-Cas9-kti13, where the nuclease gene Cas9 is expressed by Arabidopsis ubiquitin 10 (U10) promoter. A bar gene driven by a MAS promoter was used for selection of the putative transformants with Bialaphos or phosphinothricin (FIG. 3A). A tandem array of two sgRNAs targeting KTI1 and one sgRNA targeting KTI3 was expressed by the U6 RNA promoter (FIG. 3B).

### KTI1 and KTI3 Genes are Knocked Out by CRISPR/Cas9 Mediated Gene Editing

pBAR-Cas9-kti1/kti3 was transformed into WM82 via Agrobacterium-mediated transformation (Plant Transformation Facility at Iowa State University). Seventeen putative transgenic shoots were regenerated. Six shoots elongated and were transferred to rooting mediums. After further selection, they were transplanted into soil. Four lines, No. #2, #5, #11 and #17 were confirmed to be true transformants by positive amplification of the bar gene and a part of the Cas9 gene (FIGS. 3C and 3D). The gene editing events in T0 plants were identified by amplification and sequencing of DNA fragments covering the sgRNA binding sites of KTI1 and KTI3. The double peaks in the sequencing chromatograms suggest that both KTI1 and KTI3 genes were mutated and resulted in heterozygous alleles in the edited plant cells (FIG. 3E). T0 seeds were harvested from TO lines #2, #5, #11 and #17. Four T0 seeds of each line were randomly picked for DNA extraction and genotyping of the KTI1 and KTI3 genes via PCR amplification and DNA sequencing. The KTI1 gene editing was completed and resulted in homozygous mutant alleles in all tested T0 seeds of the four lines. In addition, an identical gene editing pattern in KTI3 was detected in all tested T0 seeds, in which a small DNA fragment (66 bp) between two sgRNAs was lost after the gene editing (FIG. 4A-4C). Homozygous KTI3 mutant alleles were only detected in T0 seeds from 2-3, #5-4, #11-2, and #11-4 (FIG. 4D-4G). The gene editing patterns in KTI3 included both small deletions and insertions that all resulted in frameshift mutations in KTI3.

### Sequences for Wild-Type and Mutant KTI1 and KTI3 Genes and Gene Products

KTI1 Wild-type (Williams 82 Cultivar)—Glycine max GeneID:100305855 NM_001250776 (version NM_001250776.3)

(SEQ ID NO: 1)
TGATAAAATATTGAGTTTCTTTTAGTGGAACTATTTGTCAAAATGTGAACACCTGGA
TATGAAAAGGCATCTTAGGTAGATGATATGATGCGATAGAACGTAAAAGAAAAATG
AGAAATGTTGATGAGAGGTTAAAAATACCCTTCATAACAAGCACACATCTATAAGT
AGTCTTATTCACCCAACAACGTTGCTTATTCACGCAACTAAATAAGAAATGAAGAGT
ACTATCTTCTTTGCTCTCTTTCTAGTTTGTGCCTTCACCATCTCATACCTGCCTTCAGC
CACCGCTCAGTTCGTGCTCGACACTGATGATGATCCTCTTCAAAACGGTGGCACATA
CTATATGTTGCCAGTTATGAGAGGAAAGGGCGGTGGAATAGAAGTAGATTCAACTG
GAAAAGAAATATGCCCTCTCACTGTTGTGCAATCACCCAATGAGCTCGATAAGGGG
ATTGGACTAGTCTTTACATCTCCATTACATGCCCTTTTTATCGCCGAAGGCTATCCTT
TGAGCATTAAGTTTGGTTCATTTGCAGTTATAACGCTGTGTGCCGGCATGCCTACTG
AGTGGGCTATTGTGGAGAGAGAGGGTCTACAAGCTGTTAAACTTGCTGCACGCGAT
ACAGTAGATGGTTGGTTTAATATTGAGAGAGTTTCCCGTGAATACAATGACTATAAG
CTTGTGTTCTGTCCACAGCAAGCTGAAGATAACAAATGCGAGGATATTGGGATTCAG
ATCGACGATGATGGAATCAGGCGTTTAGTGCTGTCTAAGAACAAACCATTAGTGGTT
CAGTTTCAGAAATTTAGATCATCAACTGCATGAAGCGTGAAAAATCACGTGCTTTCT
TGTTAAGAGAGACAAGTGTACGTAAGACTAAATAAATGCTTTGGCTAATAATAAATT
GGTTACAGACTAAATAAAATAATCAAAGCTTATGCCTTCCCCCAAAGGCTGAATGCA
AAAAA
Wild-type KTI1 protein (Williams 82 Cultivar) NP_001237705 (version
NP_001237705.1)
(SEQ ID NO: 2)
MKSTIFFALFLVCAFTISYLPSATAQFVLDTDDDPLQNGGTYYMLPVMRGKGGGIEVDS
TGKEICPLTVVQSPNELDKGIGLVFTSPLHALFIAEGYPLSIKFGSFAVITLCAGMPTEWAI
VEREGLQAVKLAARDTVDGWFNIERVSREYNDYKLVFCPQQAEDNKCEDIGIQIDDDGI
RRLVLSKNKPLVVQFQKFRSSTA
KTI3 Wild-type (Williams 82 Cultivar)-Glycine Max NM_001251682 (version
NM_001251682.2) (mRNA)
(SEQ ID NO: 3)
ATTCACACAACTAACTAAGAAAGTCTTCCATAGCCCCCCAAAAATGAAGAGCACCA
TCTTCTTTGCTCTCTTTCTCTTTTGTGCCTTCACCACCTCATACCTACCTTCAGCCATC
GCTGATTTCGTGCTCGATAATGAAGGTAACCCTCTTGAAAATGGTGGCACATATTAT
ATCTTGTCAGACATAACAGCATTTGGTGGAATAAGAGCAGCCCCAACGGGAAATGA
AAGATGCCCTCTCACTGTGGTGCAATCTCGCAATGAGCTCGACAAAGGGATTGGAA
CAATCATCTCGTCCCCATATCGAATCCGTTTTATCGCCGAAGGCCATCCTTTGAGCCT
TAAGTTCGATTCATTTGCAGTTATAATGCTGTGTGTTGGAATTCCTACCGAGTGGTCT
GTTGTGGAGGATCTACCAGAAGGACCTGCTGTTAAAATTGGTGAGAACAAAGATGC
AATGGATGGTTGGTTTAGACTTGAGAGAGTTTCTGATGATGAATTCAATAACTATAA
GCTTGTGTTCTGTCCACAGCAAGCTGAGGATGACAAATGTGGGGATATTGGGATTAG
TATTGATCATGATGATGGAACCAGGCGTTTGGTGGTGTCTAAGAACAAACCGTTAGT
GGTTCAGTTTCAAAAACTTGATAAAGAATCACTGGCCAAGAAAAATCATGGCCTTTC
TCGCAGTGAGTGAGACACAAGTGTGAGAGTACTAAATAAATGCTTTGGTTGTACGA
AATCATTACACTAAATAAAATAATCAAAGCTTATATATGCCTTCCGCTAAGGCCGAA
TGCAAAGAAATTGGTTCTTTCTCGTTATCTTTTGCCACTTTTACTAGTACGTATTAAT
TACTACTTAATCATCTTTGTTTACGGCTTCATTATATCC
Wild-type KTI3 protein (Williams 82 Cultivar) NP_001238611 (version
NP_001238611.2)
(SEQ ID NO: 4)
KSTIFFALFLFCAFTTSYLPSAIADFVLDNEGNPLENGGTYYILSDITAFGGIRAAPTGNER
CPLTVVQSRNELDKGIGTIISSPYRIRFIAEGHPLSLKFDSFAVIMLCVGIPTEWSVVEDLP
EGPAVKIGENKDAMDGWFRLERVSDDEFNNYKLVFCPQQAEDDKCGDIGISIDHDDGT
RRLVVSKNKPLVVQFQKLDKESLAKKNHGLSRSE
KTI1 mutant DNA Has 66 bp deletion. See FIG. 4A.
(SEQ ID NO: 5)
TGATAAAATATTGAGTTTCTTTTAGTGGAACTATTTGTCAAAATGTGAACACCTGGA
TATGAAAAGGCATCTTAGGTAGATGATATGATGCGATAGAACGTAAAAGAAAAATG
AGAAATGTTGATGAGAGGTTAAAAATACCCTTCATAACAAGCACACATCTATAAGT
AGTCTTATTCACCCAACAACGTTGCTTATTCACGCAACTAAATAAGAAATGAAGAGT
ACTATCTTCTTTGCTCTCTTTCTAGTTTGTGCCTTCACCATCTCATACCTGCCTTCAGC
CACCGCTCAGTTCGTGCTCGACACTGATGATGATCCTCTTCAAAACGGTGGCACATA
CTATATGTTGCCAGTTATGAGAGGAAAGGGCGGTGGAATAGAAGTAGATTCAACTG
GAAAAGAAATATGCCCTCTCACTGTTGTGCAATCACCCAATGAGCTCGATAAGGGG
ATTGGACTAGTCTTTACATCTCCATTACATGCCCTTTTTATCGCCGAAGGCTATCCTT
TGAGCATTAAGTTTGGTTCATTTGCAGTTATAACGCTGTGTGCCGGCATGCCTACTG
GTTGGTTTAATATTGAGAGAGTTTCCCGTGAATACAATGACTATAAGCTTGTGTTCT
GTCCACAGCAAGCTGAAGATAACAAATGCGAGGATATTGGGATTCAGATCGACGAT
GATGGAATCAGGCGTTTAGTGCTGTCTAAGAACAAACCATTAGTGGTTCAGTTTCAG
AAATTTAGATCATCAACTGCATGAAGCGTGAAAAATCACGTGCTTTCTTGTTAAGAG
AGACAAGTGTACGTAAGACTAAATAAATGCTTTGGCTAATAATAAATTGGTTACAG
ACTAAATAAAATAATCAAAGCTTATGCCTTCCCCCAAAGGCTGAATGCAAAAAA
KTI3 mutant 1 (2-3) DNA (Has a 69 bp insertion (italics) and a 22 bp
deletion (see FIG. 4D-4E)
(SEQ ID NO: 6)
ATTCACACAACTAACTAAGAAAGTCTTCCATAGCCCCCCAAAAATGAAGAGCACCA
TCTTCTTTGCTCTCTTTCTCTTTTGTGCCTTCACCACCTCATACCTACCTTCAGCCATC
GCTGATTTCGTGCTCGATAATGAAGGTAACCCTCTTGAAAATGGTGGCACATATTAT
ATCTTGTCAGACATAACAGCATTTGGTGGAATAAGAGCAGCCCCAACGGGAAATGA
AAGATGCCCTCTCACTGTGGTGCAATCTCGCAATGAGCTCGACAAAGGGATTGGAA
CAATCATCTCGTCCCCATATCGAATCCGTTTTATCGCCGAAGGCCATCCTTTGAGCCT
TAAGTTCGATTCATTTGCAGTTATAATTCCAACACACAGCAGTTATAATTCCAACAC
ACAGCATTATAATTGCAAATGAATCGAACTTAATCCTTTGAGTGGTCTGTTGTGGAG
GATCTACCAGAAGGACCTGCTGTTAAAATTGGTGAGAACAAAGATGCAATGGATGG
TTGGTTTAGACTTGAGAGAGTTTCTGATGATGAATTCAATAACTATAAGCTTGTGTTC
TGTCCACAGCAAGCTGAGGATGACAAATGTGGGGATATTGGGATTAGTATTGATCAT
GATGATGGAACCAGGCGTTTGGTGGTGTCTAAGAACAAACCGTTAGTGGTTCAGTTT
CAAAAACTTGATAAAGAATCACTGGCCAAGAAAAATCATGGCCTTTCTCGCAGTGA
GTGAGACACAAGTGTGAGAGTACTAAATAAATGCTTTGGTTGTACGAAATCATTACA
CTAAATAAAATAATCAAAGCTTATATATGCCTTCCGCTAAGGCCGAATGCAAAGAA
ATTGGTTCTTTCTCGTTATCTTTTGCCACTTTTACTAGTACGTATTAATTACTACTTAA
TCATCTTTGTTTACGGCTTCATTATATCC
KTI3 mutant 2 (5-4) DNA (A single C deletion (C₃₉₁) (see FIG. 4D and 4F)
(SEQ ID NO: 7)
ATTCACACAACTAACTAAGAAAGTCTTCCATAGCCCCCCAAAAATGAAGAGCACCA
TCTTCTTTGCTCTCTTTCTCTTTTGTGCCTTCACCACCTCATACCTACCTTCAGCCATC
GCTGATTTCGTGCTCGATAATGAAGGTAACCCTCTTGAAAATGGTGGCACATATTAT
ATCTTGTCAGACATAACAGCATTTGGTGGAATAAGAGCAGCCCCAACGGGAAATGA
AAGATGCCCTCTCACTGTGGTGCAATCTCGCAATGAGCTCGACAAAGGGATTGGAA
CAATCATCTCGTCCCCATATCGAATCCGTTTTATCGCCGAAGGCCATCCTTTGAGCCT
TAAGTTCGATTCATTTGCAGTTATAATGCTGTGTGTTGGAATTCCTAC*GAGTGGTCT
GTTGTGGAGGATCTACCAGAAGGACCTGCTGTTAAAATTGGTGAGAACAAAGATGC
AATGGATGGTTGGTTTAGACTTGAGAGAGTTTCTGATGATGAATTCAATAACTATAA
GCTTGTGTTCTGTCCACAGCAAGCTGAGGATGACAAATGTGGGGATATTGGGATTAG
TATTGATCATGATGATGGAACCAGGCGTTTGGTGGTGTCTAAGAACAAACCGTTAGT
GGTTCAGTTTCAAAAACTTGATAAAGAATCACTGGCCAAGAAAAATCATGGCCTTTC
TCGCAGTGAGTGAGACACAAGTGTGAGAGTACTAAATAAATGCTTTGGTTGTACGA
AATCATTACACTAAATAAAATAATCAAAGCTTATATATGCCTTCCGCTAAGGCCGAA
TGCAAAGAAATTGGTTCTTTCTCGTTATCTTTTGCCACTTTTACTAGTACGTATTAAT
TACTACTTAATCATCTTTGTTTACGGCTTCATTATATCC
KTI3 mutant 3 (5-26) DNA (38 bp deletion (see FIG. 4D and 4H)
(SEQ ID NO: 8)
ATTCACACAACTAACTAAGAAAGTCTTCCATAGCCCCCCAAAAATGAAGAGCACCA
TCTTCTTTGCTCTCTTTCTCTTTTGTGCCTTCACCACCTCATACCTACCTTCAGCCATC
GCTGATTTCGTGCTCGATAATGAAGGTAACCCTCTTGAAAATGGTGGCACATATTAT
ATCTTGTCAGACATAACAGCATTTGGTGGAATAAGAGCAGCCCCAACGGGAAATGA
AAGATGCCCTCTCACTGTGGTGCAATCTCGCAATGAGCTCGACAAAGGGATTGGAA
CAATCATCTCGTCCCCATATCGAATCCGTTTTATCGCCGAAGGCCATCCTTTGAGCCT
TAAGTTCGATTCATTTGCAGTTATAATGCTGTGTGTTGGAATTCCTACCGA*AGGACC
TGCTGTTAAAATTGGTGAGAACAAAGATGCAATGGATGGTTGGTTTAGACTTGAGA
GAGTTTCTGATGATGAATTCAATAACTATAAGCTTGTGTTCTGTCCACAGCAAGCTG
AGGATGACAAATGTGGGGATATTGGGATTAGTATTGATCATGATGATGGAACCAGG
CGTTTGGTGGTGTCTAAGAACAAACCGTTAGTGGTTCAGTTTCAAAAACTTGATAAA
GAATCACTGGCCAAGAAAAATCATGGCCTTTCTCGCAGTGAGTGAGACACAAGTGT
GAGAGTACTAAATAAATGCTTTGGTTGTACGAAATCATTACACTAAATAAAATAATC
AAAGCTTATATATGCCTTCCGCTAAGGCCGAATGCAAAGAAATTGGTTCTTTCTCGT
TATCTTTTGCCACTTTTACTAGTACGTATTAATTACTACTTAATCATCTTTGTTTACGG
CTTCATTATATCC
KTI3 mutant DNA 6 bp deletion
(SEQ ID NO: 9)
ATTCACACAACTAACTAAGAAAGTCTTCCATAGCCCCCCAAAAATGAAGAGCACCA
TCTTCTTTGCTCTCTTTCTCTTTTGTGCCTTCACCACCTCATACCTACCTTCAGCCATC
GCTGATTTCGTGCTCGATAATGAAGGTAACCCTCTTGAAAATGGTGGCACATATTAT
ATCTTGTCAGACATAACAGCATTTGGTGGAATAAGAGCAGCCCCAACGGGAAATGA
AAGATGCCCTCTCACTGTGGTGCAATCTCGCAATGAGCTCGACAAAGGGATTGGAA
CAATCATCTCGTCCCCATATCGAATCCGTTTTATCGCCGAAGGCCATCCTTTGAGCCT
TAAGTTCGATTCATTTGCAGTTATAATGCTGTGTGTTGGAATCGAGTGGTCTGTTGTG
GAGGATCTACCAGAAGGACCTGCTGTTAAAATTGGTGAGAACAAAGATGCAATGGA
TGGTTGGTTTAGACTTGAGAGAGTTTCTGATGATGAATTCAATAACTATAAGCTTGT
GTTCTGTCCACAGCAAGCTGAGGATGACAAATGTGGGGATATTGGGATTAGTATTGA
TCATGATGATGGAACCAGGCGTTTGGTGGTGTCTAAGAACAAACCGTTAGTGGTTCA
GTTTCAAAAACTTGATAAAGAATCACTGGCCAAGAAAAATCATGGCCTTTCTCGCAG
TGAGTGAGACACAAGTGTGAGAGTACTAAATAAATGCTTTGGTTGTACGAAATCATT
ACACTAAATAAAATAATCAAAGCTTATATATGCCTTCCGCTAAGGCCGAATGCAAA
GAAATTGGTTCTTTCTCGTTATCTTTTGCCACTTTTACTAGTACGTATTAATTACTACT
TAATCATCTTTGTTTACGGCTTCATTATATCC
KTI3 mutant 4 (11-2) and 5 (11-4) DNA 7 bp deletion (see FIG. 4D and 4G)
(SEQ ID NO: 49)
ATTCACACAACTAACTAAGAAAGTCTTCCATAGCCCCCCAAAAATGAAGAGCACCA
TCTTCTTTGCTCTCTTTCTCTTTTGTGCCTTCACCACCTCATACCTACCTTCAGCCATC
GCTGATTTCGTGCTCGATAATGAAGGTAACCCTCTTGAAAATGGTGGCACATATTAT
ATCTTGTCAGACATAACAGCATTTGGTGGAATAAGAGCAGCCCCAACGGGAAATGA
AAGATGCCCTCTCACTGTGGTGCAATCTCGCAATGAGCTCGACAAAGGGATTGGAA
CAATCATCTCGTCCCCATATCGAATCCGTTTTATCGCCGAAGGCCATCCTTTGAGCCT
TAAGTTCGATTCATTTGCAGTTATAATGCTGTGTGTTGGAATGAGTGGTCTGTTGTGG
AGGATCTACCAGAAGGACCTGCTGTTAAAATTGGTGAGAACAAAGATGCAATGGAT
GGTTGGTTTAGACTTGAGAGAGTTTCTGATGATGAATTCAATAACTATAAGCTTGTG
TTCTGTCCACAGCAAGCTGAGGATGACAAATGTGGGGATATTGGGATTAGTATTGAT
CATGATGATGGAACCAGGCGTTTGGTGGTGTCTAAGAACAAACCGTTAGTGGTTCAG
TTTCAAAAACTTGATAAAGAATCACTGGCCAAGAAAAATCATGGCCTTTCTCGCAGT
GAGTGAGACACAAGTGTGAGAGTACTAAATAAATGCTTTGGTTGTACGAAATCATT
ACACTAAATAAAATAATCAAAGCTTATATATGCCTTCCGCTAAGGCCGAATGCAAA
GAAATTGGTTCTTTCTCGTTATCTTTTGCCACTTTTACTAGTACGTATTAATTACTACT
TAATCATCTTTGTTTACGGCTTCATTATATCC

### TI Content and Activity Dramatically Declined in the Edited Soybean Seeds

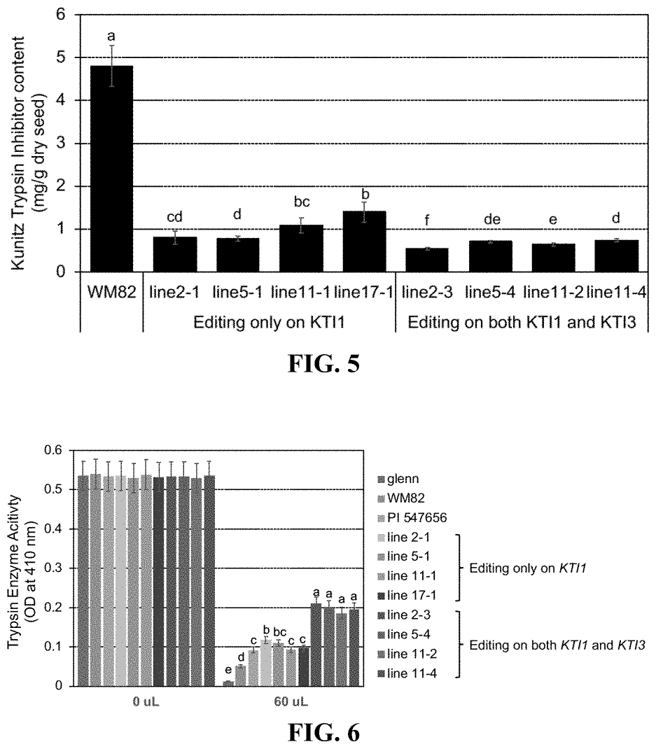

T0 seeds were also used for quantification of the KTI content by using a HPLC-based approach (Rosso, Shang et al. 2018). The tested seeds of #2-3, #5-4, #11-2, and #11-4, which carried mutations on both KTI1 and KTI3 genes had the lowest KTI content (FIG. 5). The tested seeds of #2-1, #5-1, #11-1, and #17-1, with only the KTI1 mutation, also had lower KTI content than the wild-type WM82 seeds (FIG. 5). The KTI content in other genotyped seeds with editing only on KTI1 was also lower than that in WM82 seeds (data not shown). Applicant further tested the trypsin inhibition activity (TIA) using crude protein extracts from the T0 seeds. As shown in FIG. 6, the crude proteins of seeds with mutant kti1 and kti3 had the lowest TIA (FIG. 6). The seeds with mutant kti1 only also had reduced TIA (FIG. 6) in comparison with WM82 and Glenn (a commercial soybean cultivar as a control). The KTI content and TIA were ranked in order as: kti1/3 double mutant<kti1 single mutant≤PI 547656 (low TI accession)<WM82<Glenn. Taken together, Applicant concluded that KTI1 and KTI3 are two major genes responsible for the KTI content and TIA in soybean seeds. Therefore, knockout of KTI1 and KTI3 reduced the KTI content and impaired the TIA in soybean seeds.

The edited KTI1 gene lost 66 bp that may result in mutant proteins with deletion of 22 amino acids. Truncated KTI1 may still possess some TIA. To rule out this possibility, Applicant also tested the TIA of truncated KTI1Δ22aa protein in vitro. To this end, Applicant cloned the open reading frames of KTI1Δ66 bp and wild-type KTI1 and KTI3 into a protein expression vector, in which a 6×His tag is fused to C-terminus of the expressed proteins. The purified proteins were subjected to a TIA assay which showed that while KTI1 and KTI3 both could inhibit trypsin activity, the truncated KTI1Δ22aa failed to suppress trypsin activity (FIGS. 8A and 8B). Therefore, the new kti1 allele (KTI1Δ66 bp) encodes a truncated protein that loses its TI function.

### Knockout KTI1 and KTI3 Did not Affect Plant Growth and Maturity Period Days of Soybean

To examine whether the mutant kti1/3 could significantly affect plant growth and maturity period days of soybean, Applicant planted T0 seeds of line #2 and #5, and WM82 in a greenhouse. By Bialaphos-mediated screening, Applicant classified the T1 plants from line #2 and #5 as transgene-free plants or transgenic plants. Applicant measured the agronomic traits of the transgenic plants including plant height, the number of main branches per plant, number of pods bearing branches, number of pods, leaf length, leaf width and petiole length. There was no significant difference in terms of all measured agronomic traits among the plants of WM82, Line 2 and Line 5 (Table 1). Applicant also measured the maturity period days of the soybean plants by recording the dates from planting to beginning bloom (R1), to beginning pod (R3), to beginning seed (R5), to full seed (R6), to maturity (R8), and the total lifespan (from planting to maturity). There were no remarkable differences in terms of R1, R3, R5, R6, R8 and total life span among all tested plants (Table 1). Therefore, Applicant conclude that knockout of KTI1 and KTI3 did not alter plant growth or the maturity period of soybean lines tested.

TABLE 1
Knockout of KTI1 and KTI3 does not alter plant growth
indicator and maturity period days of cv. WM82
Plant Growth Indicator

| | Plant | Main branches | Pod bearing | No. of Pods | Leaf | Leaf | Petiole |
|---|---|---|---|---|---|---|---|
| Genotypes | height | per plant | branches | per plant | Length | Width | length |
| WM82 | 29.5 ± 2.2 | 2.2 ± 0.4 | 16.0 ± 1.6 | 30.0 ± 2.9 | 11.2 ± 1.1 | 6.8 ± 0.8 | 12.4 ± 1.5 |
| Line 2 | 29.2 ± 1.2 | 2.4 ± 0.5 | 16.4 ± 1.8 | 29.4 ± 3.8 | 11.6 ± 1.8 | 7.0 ± 1.0 | 11.4 ± 1.9 |
| Transgenic | | | | | | | |
| Line 2 Non- | 29.8 ± 1.2 | 2.5 ± 0.5 | 16.6 ± 1.7 | 30.7 ± 3.2 | 11.3 ± 1.5 | 7.1 ± 1.0 | 11.9 ± 2.0 |
| transgenic | | | | | | | |
| Line 5 | 29.1 ± 1.3 | 2.2 ± 0.4 | 17.2 ± 1.6 | 28.6 ± 3.4 | 11.4 ± 1.3 | 7.2 ± 0.8 | 11.2 ± 2.4 |
| Transgenic | | | | | | | |
| Line 5 Non- | 29.0 ± 1.5 | 2.4 ± 0.3 | 16.9 ± 1.8 | 29.1 ± 3.5 | 11.0 ± 1.4 | 6.9 ± 0.8 | 11.6 ± 1.8 |
| transgenic | | | | | | | |

Maturity Period Days

| Genotype | Plant to R1 | R1 to R3 | R3 to R5 | R5 to R6 | R6 to R8 | Planting to R8 |
|---|---|---|---|---|---|---|
| WM82 | 43.8 ± 3.1 | 23.6 ± 2.1 | 18.6 ± 2.1 | 30.8 ± 2.6 | 19.6 ± 2.7 | 136.4 ± 4.7 |
| Line 2 | 40.8 ± 3.3 | 22.4 ± 2.7 | 22.4 ± 2.7 | 32 ± 2.5 | 21.4 ± 2.3 | 136. ± 6.1 |
| Transgenic | | | | | | |
| Line 2 Non- | 42.1 ± 3.8 | 23.0 ± 3.0 | 23.0 ± 3.0 | 19.4 ± 3.1 | 20.1 ± 2.3 | 136.0 ± 5.5 |
| transgenic | | | | | | |
| Line 5 | 41.0 ± 4.1 | 20.6 ± 2.9 | 20.6 ± 2.9 | 20.8 ± 2.8 | 20.0 ± 2.1 | 133.4 ± 8.9 |
| Transgenic | | | | | | |
| Line 5 Non- | 41.7 ± 4.4 | 22.5 ± 2.8 | 22.5 ± 2.6 | 20.5 ± 2.6 | 19.9 ± 2.4 | 135.3 ± 6.7 |
| transgenic | | | | | | |

R1: From planting to beginning bloom; R3: beginning bloom to beginning pod; R5: beginning pod to beginning seed; R6: beginning seed to full seed; R8: full seed to maturity. For each genotype, 5 plants were utilized for measuring plant growth indicators and maturity period days. An ANOVA test was employed here for statistical analysis.

### Development of Molecular Markers for Selection of the Kti1 and Kti3 Alleles

A double homozygous kti1 and kti3 mutant plant #5-26 that did not carry the Cas9 transgene was selected from T1 generation plants (FIGS. 4A, 4C, 4D, and 4H). The ‘transgene-free’ soybean plants can be used to breed the low TI trait into other elite soybean cultivars. In order to co-select the kti1 and kti3 mutant alleles in the derived progenies, Applicant attempted to develop co-dominate molecular markers that can distinguish between wild-type KTI1/KTI3 and mutant alleles.

In the genome of #5-26, the mutant allele of kti1 had a 66 bp deletion. Applicant designed three PCR primers, ZW1, ZW2 and ZW3 (FIG. 7A and Table 2). ZW1 was a common reverse primer that can bind to the same region in both KTI1 and kti1 alleles, while ZW2 and ZW3 were both forward primers binding to unique sequences of KTI1 and kti1 alleles, respectively (FIG. 7A). Soybean cultivars that carry the wild-type KTI1 gene (WM82) amplified a 180 bp DNA fragment when hybridized with ZW1 and ZW2, but failed to amplify any fragments when hybridized with ZW1 and ZW3. On the contrary, the soybean lines carrying a homozygous kti1 mutant allele (#5-26) amplified a 134 bp DNA fragment with ZW1 and ZW3, but failed to amplify any fragments with ZW1 and ZW2 (FIG. 7B).

Applicant further tested these PCR primers by amplifying DNA fragments from two T1 plants that were genotyped by DNA sequencing. Line #5-9 had homozygous kti1 alleles and heterozygous KTI3/kti3 alleles, where the kti3 allele was identical to the one in #5-26. Line #2-30 had homozygous kti3 alleles and heterozygous KTI1/kti1 alleles, where the kti1 allele was identical to the one in #5-26. As shown in (FIG. 7B), PCR amplification with the different combinations of ZW1, ZW2, ZW3, ZW4, ZW5 and ZW6 can accurately identify the KTI1/kti1 and KTI3/kti3 genotypes of #5-9 and #2-30 (FIG. 7B). Therefore, Applicant successfully developed molecular markers to select the kti1 and kti3 mutant alleles generated by CRISPR/Cas9 mediated mutagenesis. These molecular markers can assist in the breeding selection of low TI soybean plants harboring kti1/3.

To simplify the procedure of marker-aided selection, Applicant tested a gel-electrophoresis-free protocol that can be implemented for high throughput screening of progenies derived from a cross between a soybean cultivar carrying wild-type KTI1/3 and one carrying the kti1/3 mutant. In brief, all PCR products as described above were mixed with 1×SYBR Green and heated at 75° C. for 10 mins and then visualized under UV light. As shown in (FIG. 7B), the fluorescent signals were the indications of positive amplifications in WM82 with primers ZW1/ZW2 and ZW4/ZW5, while in #5-26, the fluorescent signals can only be observed with primers ZW1/ZW3 and ZW4/ZW6 (Hirotsu, Murakami et al. 2010). Therefore, Applicant identified the homozygous kti1 and kti3 alleles by directly staining the PCR products without the need of gel electrophoresis, which can significantly reduce the cost of labor and time.

### Discussion

In this study, Applicant optimized a CRISPR/cas9-vector for genome editing in soybean (FIG. 3A). The modified vector allowed us to simultaneously knock out two seed specific KTI genes (KTI1 and KTI3). The kti1/3 mutant plants grew normally in greenhouse conditions, and the seeds of kti1/3 mutant had dramatically reduced KTI content and TI activities in comparison with wild type seed of WM82.

Soybean is one of the important sources of protein for animal and human consumption. However, in their evolution, soybeans have developed diverse defense components to protect seeds from being eaten by insects and animals including trypsin inhibitor, phytate acid, and raffinose family of oligosaccharides (RFOs). In the agricultural practice, the anti-nutritional and biologically active factors are responsible for reduced feed efficiency when raw soybeans are fed to animals. Therefore, it is of great significance to increase feed efficiency, especially the protein digestibility via assembling gene function exploration, application, and advanced genetically engineering together into the soybean industry. Proteinaceous plant trypsin inhibitors are a diverse family of (poly)peptides that play diverse roles in plant growth such as maintaining physiological homeostasis and serving the innate defense machinery (Li, Brader et al. 2008, Junker, Zeissig et al. 2012, Arnaiz, Talavera-Mateo et al. 2018, Zhao, Ullah et al. 2019). Since TI proteins exert direct effects on pests and herbivores by interfering with their physiology, any food containing TI proteins will be avoided by these organisms. In alfalfa, the trypsin inhibitors Msti-94 and Msti-16 were demonstrated to act as a stomach poison, significantly reducing the survival and reproduction rates of aphid (Zhao, Ullah et al. 2019). Mutant plants with reduced TI are usually more susceptible to pests. In wheat, α-amylase/trypsin inhibitors (ATIs) CM3 and 0.19 were identified as pest-resistance molecules, activating innate immune responses in monocytes, macrophages, and dendritic cells (Junker, Zeissig et al. 2012). For example, the Arabidopsis lines containing silenced atkti4 and atkti5 were found to have a higher susceptibility to T. urticae (Spider mite) than wild-type plants (Arnaiz, Talavera-Mateo et al. 2018). RNAi silencing of the AtKTI01 gene resulted in enhanced lesion development after infiltration of leaf tissue with the programmed cell death eliciting fungal toxin fumonisin B1 or the avirulent bacterial pathogen Pseudomonas syringae pv. tomato DC3000 carrying avrB (Li, Brader et al. 2008). Although similar defense functions have not been reported on TI genes in the soybean genome, it is reasonably suspected that certain members of the KTI gene family have comparable protecting roles for soybean plants.

As previously discussed, the high concentration of TI proteins in soybean meal restricts the function of trypsin, which causes low digestibility and reduces its nutritional value. Thus, cultivars have been developed by introgression of low TI traits into elite cultivars. Applicant previously developed a low TI line via conventional breeding: V12-4590. During field trials in 2017 and 2018, Applicant observed that this low TI line is indeed more susceptible to multiple phytopathogens such as: all races of Soybean Cyst Nematode (SCN) (Heterodera glycines), Stem Canker (Diaporthe aspalathi), Cercospora leaf blight (Cercospora kukuchii), Soybean vein necrosis virus, and Downy Mildew (Peronospora manshurica) (Zhang, unpublished data). This suggests that that the soybean KTI genes that are negatively selected do indeed have a role in plant immunity. It is also possible that some plant immunity related genes that genetically link with KTI genes are negatively selected during the breeding process. As shown in FIG. 1, at least 13 KTI genes are clustered in a small region on chromosome 8. Interestingly, Applicant also identified a putative TGACG-Binding (TGA) transcription factor (TF) that is tightly linked to the KTI gene cluster at chromosome 8. Arabidopsis TGA TFs play a positive role in systemic acquired resistance (SAR) that is crucial in plant immunity (Hussain, Sheikh et al. 2018). Therefore, breeding of low TI soybean lines resulting in the loss or mutation of both of the KTI genes and the TGA TF gene, leading to an increased susceptibility of soybean challenged by phytopathogens.

The kti1/3 mutant soybean line generated via CRISPR/Cas9-mediated mutagenesis is an isogenic line of wild type WM82 (Table 1) Therefore, it will be an ideal test subject to see if KTI1/3 has a direct role in plant immunity. Since KTI1/3 were almost only expressed in seeds (FIG. 2) (Gillman, Kim et al. 2015), the knockout of these two genes may not interfere with plant immunity in non-seed tissue, which deserves to be further investigated in the future.

In this study, Applicant identified a kti1/3 double homozygous mutant along with kti1 and kti3 single homozygous mutants. The kti1/3 double homozygous mutant has the lowest KTI content and trypsin inhibition activity (FIGS. 5 and 6). Therefore, it can be determined that (Hussain, Sheikh et al. 2018) KTI1 and KTI3 synergistically contribute to the KTI content and TI activity in soy proteins. The previous report suggests that the soybean line carrying natural mutations of kti1 and kti3 has increased BBTI content (Gillman, Kim et al. 2015). It is unclear if the increased BBTI content is caused by un-intentional selection during the breeding process or if the expression of BBTI genes is increased because of the mutations of two KTI genes. Therefore, it will be interesting to test the BBTI content and activity in the seed proteins of the kti1/3 mutant generated in this study.

Despite the fact that the CRISPR/Cas9 technique has been successfully utilized to generate various soybean mutants (Jacobs, LaFayette et al. 2015), the current agrobacterium-mediated soybean transformation protocol is inefficient and genotype dependent. This limits the wide implementation of CRISPR/Cas9 technique in soybean breeding programs (Yamada, Takagi et al. 2012). The soybean transformation protocol employs Bialaphos as the selection agent (Luth, Warnberg et al. 2015). The Bar gene is used as the selection marker gene and encodes a phosphinothricin acetyltransferase protein that can confer the transformants' resistance to bialaphos. It has been reported that the Bargene expression must be fine-tuned in order to successfully select true transgenic plants (Testroet, Lee et al. 2017). The original CRISPR/Cas9 vector, pCut, has used a MAS (mannopine synthase) promoter to express the Bar gene (Peterson, Haak et al. 2016). However, for unknown reasons, the vector does not work well, even in Arabidopsis thaliana (Liu and Zhao, unpublished data).

With the intention of improving the transformation system, Applicant modified the Bialaphos selection vector in the pMU3T (Liu, Miao et al. 2016). Specifically, Applicant replaced the Kanamycin selection marker gene with the Bar gene, whose expression was driven by a MAS promoter (FIG. 3A). The MAS promoter is known to be most active in the roots of emerging seedlings and very active in the cotyledons and lower leaves (Langridge, Fitzgerald et al. 1989). Despite the MAS promoter having a lower level of expression than p35S, populations of transformants created with this promoter show normally distributed expression levels (Perez-Gonzalez and Caro 2019). Thus, the MAS promoter can be used for functional screening of positive transformants in both of Applicant's shoot re-generation and rooting medium supplemented with bialaphos.

It is noteworthy that, before the initiation of stable transformation, Applicant evaluated the effectiveness of gRNAs by using a convenient Agrobacterium-mediated transient assay method (Wang et al, the company manuscript submitted for review). As soybean plants have a long-life cycle (4-6 months), the estimation of the gRNAs' effectiveness helps to avoid the waste of time and enhance the possibility of obtaining authentic gene-edited plants.

Although the soybean cultivars with natural variations on either KTI1 (PI 68679) or KTI3 (PI 542044) have been discovered, conventional breeding to develop new cultivars stacking with two mutant alleles via crossing will take a long time. In addition, linkage drag might lead to interference with the functions of genes located at the flanking sequences of mutant kti1 or kti3. The limited genetic background of natural kti1 and kti3 mutants may also reduce the genetic diversity of soybean breeding lines with low TI trait, and it can be difficult to stack low TI trait with a bundle of various, desirable traits. In the present study, the kti1/3 mutant line was created using cv. WM82, which has a genetic background distinct from accessions that harbor natural kti1 and/or kti3 mutations. Therefore, it offers a new recourse for breeding low TI traits in soybean practice.

Current soybean transformation protocol is genotype dependent, and only a few cultivars (WM82, Jack, Thorne, etc.) can be efficiently transformed (Yamada, Takagi et al. 2012). A mutant allele must be created in those transformable cultivars and bred into other elite cultivars via marker-assisted selection (MAS). Thus, creating a mutant allele tagged with convenient molecular markers is essential for MAS (Hasan, Choudhary et al. 2021). CRIPSR/Cas9-based genome editing can introduce small deletions/insertions to targeted genes, enabling us to develop molecular markers based on the sequences of the insertion and deletion mutation regions. In this study, Applicant tested using single sgRNA and two sgRNAs for generation of mutagenesis on KTI1 and KTI3, respectively (FIG. 3B). Interestingly, Applicant observed that all genotyped mutant lines carried an identical gene editing pattern of the kti1 gene, where 66 nucleotides between the two gRNAs were deleted. The homozygous kti1 allele can be identified in all tested seeds of the T0 generation, while homozygous kti3 alleles were identified in some of those genotyped T0 seeds (FIG. 4A-41I). Therefore, it is possible that two sgRNAs are more efficient for triggering the gene editing events in early generations of transgenic plants.

MAS has been widely implemented in plant breeding including soybean programs (Hasan, Choudhary et al. 2021). The selection marker of kti3 has been developed based on its natural mutant allele, but the molecular marker for the natural mutation of kti1 in PI 68679 is still not available (Gillman, Kim et al. 2015). Therefore, it is challenging to breed the natural kti1/3 mutant alleles into a new cultivar via MAS. In this study, Applicant created co-dominant markers that can distinguish between the wild and mutant alleles of KTI1/KTI3 and kti1/kti3 based on small deletions created by CRISPR/Cas9 machinery (FIG. 7A-7B). In addition, a simple gel-electrophoresis-free method can be used to identify plants carrying mutant kti1 and kti3 alleles (Hirotsu, Murakami et al. 2010). Thus, MAS makes it possible to effectively breed the new mutant kti1/3 alleles into other elite cultivars. Taken together, the whole experimental design may serve as a practical example of how to create and select mutant alleles in crop plants in the future.

### Summary

This Example at least demonstrates development of non-transgenic, low TI soybean mutant in cv. William 82. The mutant gene alleles are tagged with convenient molecular markers that are suitable for high throughput marker-aided selection. Applicant expects the low TI soybean mutant will be widely used to breed low-TI or TI-free soybean cultivars for commercial production in value-added meal industry and for stacking with other valuable agronomic traits in the future.

### REFERENCES FOR EXAMPLE 1

Arnaiz, A., L. Talavera-Mateo, P. Gonzalez-Melendi, M. Martinez, I. Diaz and M. E. Santamaria (2018). “Arabidopsis Kunitz Trypsin Inhibitors in Defense Against Spider Mites.” Front Plant Sci 9: 986.
Batal, A. B. and C. M. Parsons (2004). “Utilization of various carbohydrate sources as affected by age in the chick.” Poult Sci 83(7): 1140-1147.
Cai, Y., L. Chen, X. Liu, C. Guo, S. Sun, C. Wu, B. Jiang, T. Han and W. Hou (2018). “CRISPR/Cas9 mediated targeted mutagenesis of GmFT2a delays flowering time in soya bean.” Plant Biotechnol J 16(1): 176-185.
Cai, Y., L. Chen, X. Liu, S. Sun, C. Wu, B. Jiang, T. Han and W. Hou (2015). “CRISPR/Cas9 Mediated Genome Editing in Soybean Hairy Roots.” PLoS One 10(8): e0136064.
Chang, C. J., T. D. Tanksley, Jr., D. A. Knabe and T. Zebrowska (1987). “Effects of different heat treatments during processing on nutrient digestibility of soybean meal in growing swine.” J Anim Sci 65(5): 1273-1282.
Chen, K., Y. Wang, R. Zhang, H. Zhang and C. Gao (2019). “CRISPR/Cas Genome Editing and Precision Plant Breeding in Agriculture.” Annu Rev Plant Biol 70: 667-697.
Chen, R., Q. Xu, Y. Liu, J. Zhang, D. Ren, G. Wang and Y. Liu (2018). “Generation of Transgene Free Maize Male Sterile Lines Using the CRISPR/Cas9 System.” Front Plant Sci 9: 1180.
Chen, Y., Z. Xu, C. Zhang, X. Kong and Y. Hua (2014). “Heat-induced inactivation mechanisms of Kunitz trypsin inhibitor and Bowman-Birk inhibitor in soymilk processing.” Food Chem 154: 108-116.
Cook, D. A., A. H. Jensen, J. R. Fraley and T. Hymowitz (1988). “Utilization by growing and finishing pigs of raw soybeans of low Kunitz trypsin inhibitor content.” J Anim Sci 66(7): 1686-1691.
Cromwell, G. L., T. S. Stahly and H. J. Monegue (1991). “Amino acid supplementation of meat meal in lysine-fortified, corn-based diets for growing-finishing pigs.” J Anim Sci 69(12): 4898 4906.
Earley, K. W., J. R. Haag, O. Pontes, K. Opper, T. Juehne, K. Song and C. S. Pikaard (2006). “Gateway-compatible vectors for plant functional genomics and proteomics.” Plant J 45(4): 616-629.
Gao, W., L. Long, X. Tian, F. Xu, J. Liu, P. K. Singh, J. R. Botella and C. Song (2017). “Genome Editing in Cotton with the CRISPR/Cas9 System.” Front Plant Sci 8: 1364.
Gillman, J. D., W. S. Kim and H. B. Krishnan (2015). “Identification of a new soybean kunitz trypsin inhibitor mutation and its effect on bowman-birk protease inhibitor content in soybean seed.” J Agric Food Chem 63(5): 1352-1359.
Han, Q., C. Zhou, S. Wu, Y. Liu, L. Triplett, J. Miao, J. Tokuhisa, L. Deblais, H. Robinson, J. E. Leach, J. Li and B. Zhao (2015). “Crystal Structure of Xanthomonas AvrRxo1-ORF1, a Type III Effector with a Polynucleotide Kinase Domain, and Its Interactor AvrRxo1-ORF2.” Structure 23(10): 1900-1909.
Hasan, N., S. Choudhary, N. Naaz, N. Sharma and R. A. Laskar (2021). “Recent advancements in molecular marker-assisted selection and applications in plant breeding programmes.” J Genet Eng Biotechnol 19(1): 128.
Haun, W., A. Coffman, B. M. Clasen, Z. L. Demorest, A. Lowy, E. Ray, A. Retterath, T. Stoddard, A. Juillerat, F. Cedrone, L. Mathis, D. F. Voytas and F. Zhang (2014). “Improved soybean oil quality by targeted mutagenesis of the fatty acid desaturase 2 gene family.” Plant Biotechnol J 12(7): 934-940.
Hirotsu, N., N. Murakami, T. Kashiwagi, K. Ujiie and K. Ishimaru (2010). “Protocol: a simple gel free method for SNP genotyping using allele-specific primers in rice and other plant species.” Plant Methods 6: 12.
Hussain, R. M. F., A. H. Sheikh, I. Haider, M. Quareshy and H. J. M. Linthorst (2018). “Arabidopsis WRKY50 and TGA Transcription Factors Synergistically Activate Expression of PR1.” Frontiers in Plant Science 9.
Peterson, B. A., D. C. Haak, M. T. Nishimura, P. J. Teixeira, S. R. James, J. L. Dangl and Z. L. Nimchuk (2016). “Genome-Wide Assessment of Efficiency and Specificity in CRISPR/Cas9 Mediated Multiple Site Targeting in Arabidopsis.” PLoS One 11(9): e0162169.
Hwang, D. L., D. E. Foard and C. H. Wei (1977). “A soybean trypsin inhibitor. Crystallization and x-ray crystallographic study.” J Biol Chem 252(3): 1099-1101.
Hymowitz, T. (1986). “Genetics and breeding of soybeans lacking the Kunitz trypsin inhibitor.” Adv Exp Med Biol 199: 291-298.
Jacobs, T. B., P. R. LaFayette, R. J. Schmitz and W. A. Parrott (2015). “Targeted genome modifications in soybean with CRISPR/Cas9.” BMC Biotechnol 15: 16.
Jian, B., B. Liu, Y. Bi, W. Hou, C. Wu and T. Han (2008). “Validation of internal control for gene expression study in soybean by quantitative real-time PCR.” BMC Mol Biol 9: 59.
Jofuku, K. D. and R. B. Goldberg (1989). “Kunitz trypsin inhibitor genes are differentially expressed during the soybean life cycle and in transformed tobacco plants.” Plant Cell 1(11): 1079-1093.
Jofuku, K. D., R. D. Schipper and R. B. Goldberg (1989). “A frameshift mutation prevents Kunitz trypsin inhibitor mRNA accumulation in soybean embryos.” Plant Cell 1(5): 567.
Junker, Y., S. Zeissig, S. J. Kim, D. Barisani, H. Wieser, D. A. Leffler, V. Zevallos, T. A. Libermann, S. Dillon, T. L. Freitag, C. P. Kelly and D. Schuppan (2012). “Wheat amylase trypsin inhibitors drive intestinal inflammation via activation of toll-like receptor 4.” J Exp Med 209(13): 2395-2408.
Kim, J. and J. S. Kim (2016). “Bypassing GMO regulations with CRISPR gene editing.” Nat Biotechnol 34(10): 1014-1015.
Kunitz, M. (1945). “Crystallization of a Trypsin Inhibitor from Soybean.” Science 101(2635): 668-669.
Langridge, W. H., K. J. Fitzgerald, C. Koncz, J. Schell and A. A. Szalay (1989). “Dual promoter of Agrobacterium tumefaciens mannopine synthase genes is regulated by plant growth hormones.” Proc Natl Acad Sci USA 86(9): 3219-3223.
Li, J., G. Brader and E. T. Palva (2008). “Kunitz trypsin inhibitor: an antagonist of cell death triggered by phytopathogens and fumonisin b1 in Arabidopsis.” Mol Plant 1(3): 482-495.\
Liang, G., H. Zhang, D. Lou and D. Yu (2016). “Selection of highly efficient sgRNAs for CRISPR/Cas9-based plant genome editing.” Sci Rep 6: 21451.
Liener, I. E. (1994). “Implications of antinutritional components in soybean foods.” Critical Reviews in Food Science and Nutrition 34(1): 31-67.
Liener, I. E. (1996). “Effects of processing on antinutritional factors in legumes: the soybean case.” Arch Latinoam Nutr 44(4 Suppl 1): 48S-54S.
Liu, Y., J. Miao, S. Traore, D. Kong, Y. Liu, X. Zhang, Z. L. Nimchuk, Z. Liu and B. Zhao (2016). “SacB-SacR Gene Cassette As the Negative Selection Marker to Suppress Agrobacterium Overgrowth in Agrobacterium-Mediated Plant Transformation.” Front Mol Biosci 3: 70.
Luth, D., K. Warnberg and K. Wang (2015). “Soybean [Glycine max (L.) Merr].” Methods Mol Biol 1223: 275-284.
Nakayasu, M., R. Akiyama, H. J. Lee, K. Osakabe, Y. Osakabe, B. Watanabe, Y. Sugimoto, N. Umemoto, K. Saito, T. Muranaka and M. Mizutani (2018). “Generation of alpha-solanine-free Plant hairy roots of potato by CRISPR/Cas9 mediated genome editing of the St16DOX gene.” Physiol Biochem 131: 70-77.
Osakabe, Y., Z. Liang, C. Ren, C. Nishitani, K. Osakabe, M. Wada, S. Komori, M. Malnoy, R. Velasco, M. Poli, M. H. Jung, O. J. Koo, R. Viola and C. Nagamangala Kanchiswamy (2018). “CRISPR-Cas9-mediated genome editing in apple and grapevine.” Nat Protoc 13(12): 2844-2863.
Peterson, B. A., D. C. Haak, M. T. Nishimura, P. J. Teixeira, S. R. James, J. L. Dangl and Z. L. Nimchuk (2016). “Genome-Wide Assessment of Efficiency and Specificity in CRISPR/Cas9 Mediated Multiple Site Targeting in Arabidopsis.” PLoS One 11(9): e0162169.
Paz, M. M., J. C. Martinez, A. B. Kalvig, T. M. Fonger and K. Wang (2006). “Improved cotyledonary node method using an alternative explant derived from mature seed for efficient Agrobacterium-mediated soybean transformation.” Plant Cell Rep 25(3): 206-213.
Peng, A., S. Chen, T. Lei, L. Xu, Y. He, L. Wu, L. Yao and X. Zou (2017). “Engineering canker-resistant plants through CRISPR/Cas9-targeted editing of the susceptibility gene CsLOB1 promoter in citrus.” Plant Biotechnol J 15(12): 1509-1519.
Perez-Gonzalez, A. and E. Caro (2019). “Benefits of using genomic insulators flanking transgenes to increase expression and avoid positional effects.” Sci Rep 9(1): 8474.
Perez-Maldonado, R. A., P. F. Mannion and D. J. Farrell (2003). “Effects of heat treatment on the nutritional value of raw soybean selected for low trypsin inhibitor activity.” Br Poult Sci 44(2): 299-308.
Rosso, M. L., C. Shang, E. Correa and B. Zhang (2018). “An Efficient HPLC Approach to Quantify Kunitz Trypsin Inhibitor in Soybean Seeds.” Crop Science 58(4): 1616-1623.
Rosso, M. L., C. Shang, Q. Song, D. Escamilla, J. Gillenwater and B. Zhang (2021). “Development of Breeder-Friendly KASP Markers for Low Concentration of Kunitz Trypsin Inhibitor in Soybean Seeds.” Int J Mol Sci 22(5).
Schuler, T. H., G. M. Poppy, B. R. Kerry and I. Denholm (1999). “Potential side effects of insect-resistant transgenic plants on arthropod natural enemies.” Trends Biotechnol 17(5): 210-216.
Shao, X., S. Wu, T. Dou, H. Zhu, C. Hu, H. Huo, W. He, G. Deng, O. Sheng, F. Bi, H. Gao, T. Dong, C. Li, Q. Yang and G. Yi (2020). “Using CRISPR/Cas9 genome editing system to create MaGA20ox2 gene-modified semi-dwarf banana.” Plant Biotechnol J 18(1): 17-19.
Testroet, A., K. Lee, D. Luth and K. Wang (2017). “Comparison of transformation frequency using the bar gene regulated by the CaMV 35S or NOS promoter in Agrobacterium-mediated soybean (Glycine max L.) transformation.” In Vitro Cellular & Developmental Biology—Plant 53(3): 188-199.
Upadhyay, S. K., J. Kumar, A. Alok and R. Tuli (2013). “RNA-guided genome editing for target gene mutations in wheat.” G3 (Bethesda) 3(12): 2233-2238.
Vu, T. V., V. Sivankalyani, E. J. Kim, D. T. H. Doan, M. T. Tran, J. Kim, Y. W. Sung, M. Park, Y. J. Kang and J. Y. Kim (2020). “Highly efficient homology-directed repair using CRISPR/Cpf1-geminiviral replicon in tomato.”
Wei, C. H. (1983). “Crystallization of two cubic forms of soybean trypsin inhibitor E-I, a member of the Bowman-Birk inhibitor family.” J Biol Chem 258(15): 9357-9359.Abstract/FREE Full TextGoogle Scholar
Wu, N., Q. Lu, P. Wang, Q. Zhang, J. Zhang, J. Qu and N. Wang (2020). “Construction and Analysis of GmFAD2-1A and GmFAD2-2A Soybean Fatty Acid Desaturase Mutants Based on CRISPR/Cas9 Technology.” Int J Mol Sci 21(3).
Xu, R. F., H. Li, R. Y. Qin, J. Li, C. H. Qiu, Y. C. Yang, H. Ma, L. Li, P. C. Wei and J. B. Yang (2015). “Generation of inheritable and “transgene clean” targeted genome-modified rice in later generations using the CRISPR/Cas9 system.” Sci Rep 5: 11491.
Yamada, T., K. Takagi and M. Ishimoto (2012). “Recent advances in soybean transformation and their application to molecular breeding and genomic analysis.” Breed Sci 61(5): 480-494.
Zhao, B., D. Dahlbeck, K. V. Krasileva, R. W. Fong and B. J. Staskawicz (2011). “Computational and biochemical analysis of the Xanthomonas effector AvrBs2 and its role in the modulation of Xanthomonas type three effector delivery.” PLoS Pathog 7(12): e1002408.
Zhao, H., H. Ullah, M. R. McNeill, G. Du, K. Hao, X. Tu and Z. Zhang (2019). “Inhibitory Effects of Plant Trypsin Inhibitors Msti-94 and Msti-16 on Therioaphis trifolii (Monell) (Homoptera: Aphididae) in Alfalfa.” Insects 10(6).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. An engineered soybean plant, soybean, or both comprising:
one or more modified KTI genes thereby reducing or eliminating the expression of, amount of, and/or activity of the one or more modified KTI genes and/or gene products as compared to a wild-type or conventionally cultivated soybean plant and/or soybean therefrom.

2. The engineered soybean plant, soybean, or both of claim 1, wherein the one or more modified KTI genes are a modified KTI 1 gene, a modified KTI 3 gene, or both.

3. The engineered soybean plant, soybean, or both of claim 1, wherein the one or more modified KTI genes comprises a modified KTI3.

4. The engineered soybean plant, soybean, or both of claim 1, wherein the one or more modified KTI genes consists of a modified KTI3.

5. The engineered soybean plant, soybean, or both of claim 1, wherein the one or more modified KTI genes comprises a modified KTI1 gene comprising SEQ ID NO: 5.

6. The engineered soybean plant, soybean, or both of claim 1, wherein the one or more modified KTI genes comprises a modified KTI3 gene comprising one of SEQ ID NO: 6-9 or 49.

7. The engineered soybean plant, soybean, or both of claim 1, wherein the one or more modified KTI genes comprises a modified KTI1 gene and a modified KTI3 gene.

8. The engineered soybean plant, soybean, or both of claim 1, wherein the engineered soybean plant, soybean, or both is heterozygous for the one or more modified KTI genes.

9. The engineered soybean plant, soybean, or both of claim 1, wherein the engineered soybean plant, soybean, or both is homozygous for the one or more modified KTI genes.

10. The engineered soybean plant, soybean, or both of claim 1, wherein the expression of, amount of, and/or activity of the one or more modified KTI genes and/or gene products is decreased 0.1-1,000 fold or more.

11. The engineered soybean plant, soybean, or both of claim 1, wherein the engineered soybean plant, soybean, or both has reduced trypsin inhibitor amount and/or activity as compared to a suitable control.

12. A method comprising:
growing, harvesting, or otherwise cultivating an engineered soybean plant and/or soybean of claim 1.

13. A method of making an engineered soybean plant and/or soybean of any one of claim 1, wherein the method comprises a gene editing technique, optionally a CRISPR-Cas mediated gene editing technique.

14. A feed or food product comprising a soybean plant and/or soybean as in claim 1 or a soybean product produced therefrom.

15. A method comprising:
feeding a feed and/or food product of claim 14 to a human or non-human animal.

16. A kit for identifying soybean plants having low trypsin inhibition as compared to a suitable control, the kit comprising:
(a) a set of primers configured to amplify a region of a KTI1 mutant gene,
(b) a set of primers configured to amplify a region of a KTI3 mutant gene
(c) both (a)-(b), and
a set of primers configured to amplify a region of a wild-type or control soybean KTI1 gene, a set of primers configured to amplify a region of a wild-type or control soybean KTI3 gene, or both,
wherein the amplicon generated from the set of primers in (a) is different in size and/or sequence as compared to the amplicon generated from the set of primers configured to amplify a region of a wild-type or control soybean KTI1 gene, and
wherein the amplicon generated from the set of primers in any one of (b) are different in size and/or sequence as compared to the amplicon generated from the set of primers configured to amplify a region of a wild-type or control soybean KTI3 gene.

17. The kit of claim 16, further comprising one or more reagents suitable for isolating, preparing, amplifying, and/or sequencing nucleic acids.

18. The kit of claim 16, wherein the wild-type soybean KTI1 gene has a sequence according to SEQ ID NO: 1, the wild-type soybean KTI3 gene has a sequence according to SEQ ID NO: 3.

19. The kit of claim 16, wherein the set of primers of (a) and the set of primers configured to amplify a region of a wild-type or control soybean KTI1 gene share a common forward primer or a common reverse primer,
wherein the set of primers of (b) and the set of primers configured to amplify a region of a wild-type or control soybean KTI3 gene share a common forward primer or a common reverse primer, or
both.

20. The kit of claim 16, wherein the kit comprises one or more primers each having a sequences according to one of SEQ ID NO: 26-30.
